(19) United States
(12) Patent Application Publication
(10) Pub. No.: US 2026/0308567 A1
(43) Pub. Date: Oct. 8, 2026

(54) REMOVAL OF EXOSOMES, ECTOSOMES, MIRNAS, CIRCULATING NUCLEIC ACIDS, AND VIRAL PARTICLES WITH TRANSPLANTATION

(71) Applicant: Aethlon Medical, Inc., San Diego, CA (US)

(72) Inventors: Charles J. Fisher, Cardiff-by-the-Sea, CA (US); Steven P. Larosa, Poway, CA (US); Rosalia De Necochea Campion, Oceanside, CA (US); Paul Vallejos, Temecula, CA (US); Lee D. Arnold, Rancho Santa Fe, CA (US); Miguel Angel Pesqueira, San Diego, CA (US)

(21) Appl. No.: 19/155,517

(22) PCT Filed: Feb. 13, 2024
(86) PCT No.: PCT/US2024/015614

§ 371 Date: Aug. 11, 2025

Related U.S. Application Data
(60) Provisional application No. 63/446,156, filed on Feb. 16, 2023.
(60) Provisional application No. 63/528,611, filed on Jul. 24, 2023.

(51) Int. Cl. C12N 15/10; A61M 1/36; C12Q 1/6876

(52) U.S. Cl. CPC C12N 15/1006; A61M 1/362; A61M 1/3679; C12Q 1/6876; A61M 2202/206; C12Q 2600/178

(57) ABSTRACT

Aspects described herein concern methods for removing subcellular nanoparticles, pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, or glycolipids, or fragments thereof from organ, tissue, or cell transplant donors, organ, tissue, or cell transplant recipients, or donor organs before, during and/or after transplantation into the recipient.

### CROSS REFERENCE TO RELATED APPLICATIONS

This application claims the benefit of priority to U.S. Provisional Patent Application No. 63/446,156, filed Feb. 16, 2023 and U.S. Provisional Patent Application No. 63/528,611, filed Jul. 24, 2023, the entire contents of each of which is incorporated by reference herein.

### BACKGROUND OF THE INVENTION

### Field of the Invention

Aspects of the present invention relate to methods for improving transplantation by removing subcellular nanoparticles, a pathogen, viral particles, virus-like particles (VLPs), extracellular vesicles, such as exosomes or ectosomes, miRNAs, nucleic acids, including DNA or RNA, extracellular vesicle associated DNA, circulating extracellular DNA (ceDNA), a biomarker or mediator of tissue damage, Neutrophil extracellular traps (NETs) and their components including DNA, histones, neutrophil granule proteins or oxidant enzymes, inflammatory or immunomodulatory glycoproteins, glycolipids or lipoglycans, or fragments thereof, from donor organs, organ perfusate, a population of cells or a tissue, or from the organ or tissue donor or recipient before, during and/or after transplantation.

### Description of the Related Art

Transplantation medicine has been incredibly challenging due to the limited number of healthy, viable organs and transplant rejection, which occurs in many patients. Many in the field attribute transplant rejection to the recipient's immune response to the transplanted organ and/or unhealthy organs obtained from the donor.

Extracellular vesicles including exosomes and ectosomes are released by cells, including but not limited to mammalian cells. Methods of removing circulating extracellular vesicles, virus, and viral fragments from a patient's blood using extracorporeal devices are well known (see e.g., U.S. Pat. Nos. 9,707,333; and 10,022,483, herein expressly incorporated by reference in their entireties). The need for approaches to reduce transplant rejection and provide donor organs, cells or tissues that produce less inflammation in a recipient is manifest.

### SUMMARY OF THE INVENTION

Aspects of the present invention described herein include devices and methods for removing pathogens, viral particles, virus-like particles (VLPs), extracellular vesicles, such as exosomes or ectosomes, miRNAs, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof from an organ donor, cell or tissue donor, an organ, an organ perfusate, a population of cells or tissue being transplanted and/or the organ, cell, or tissue recipient so as to increase or improve organ, cell, or tissue viability, engraftment, and/or survival of the transplant and/or transplant recipient or to remove molecules or compositions, which are associated with tissue rejection, ischemia-reperfusion injury (IRI), organ failure, necrosis, or poor patient outcome after transplantation. Aspects of the present invention concern more generally the use of lectin (e.g., GNA) and/or antibody-based affinity methods for removal of pathogens, viral particles, virus-like particles (VLPs), extracellular vesicles, such as exosomes or ectosomes, miRNAs, nucleic acids, such as DNA or RNA, circulating extracellular DNA (ceDNA), Neutrophil extracellular traps (NETs) and their components including DNA, extracellular vesicle associated DNA, histones, neutrophil granule proteins and/or oxidant enzymes, inflammatory or immunomodulatory glycoproteins, glycolipids or lipoglycans, from a transplant donor, transplant recipient, and/or organ, tissue, an organ perfusate, or population of cells selected for transplantation. Additional embodiments concern the reintroduction of the sample obtained after a lectin and/or antibody affinity isolation or capture (e.g., after lectin and/or antibody affinity in an extracorporeal device having a substrate with an immobilized lectin and/or antibody or a column packed with lectin affinity resin (e.g., GNA)) into the subject (e.g., the donor and/or recipient of the organ, an organ perfusate, cell population, or tissue) or an isolated organ, an organ perfusate, population of cells or tissue selected for transplantation, wherein the sample obtained after the lectin and/or antibody affinity isolation or capture comprises a reduced amount of pathogens, viral particles, virus-like particles (VLPs), extracellular vesicles, such as exosomes or ectosomes, miRNAs, nucleic acids, such as DNA or RNA, circulating extracellular DNA (ceDNA), Neutrophil extracellular traps (NETs) and their components including DNA, extracellular vesicle associated DNA, histones, neutrophil granule proteins and/or oxidant enzymes, inflammatory or immunomodulatory glycoproteins, glycolipids or lipoglycans, or fragments thereof as compared to the sample prior to said lectin and/or antibody affinity isolation or capture. Additional embodiments concern selecting a subject (e.g., a transplant donor or recipient) for receiving a lectin and/or antibody affinity therapy accompanying a transplantation procedure (e.g., extracorporeal therapy), which removes pathogens, viral particles, virus-like particles (VLPs), extracellular vesicles, such as exosomes or ectosomes, miRNAs, nucleic acids, such as DNA or RNA, circulating extracellular DNA (ceDNA), Neutrophil extracellular traps (NETs) and their components including DNA, extracellular vesicle associated DNA, histones, neutrophil granule proteins and/or oxidant enzymes, inflammatory or immunomodulatory glycoproteins, glycolipids or lipoglycans, or fragments thereof. In some embodiments, the sample, such as blood or plasma from the subject (e.g., transplant donor and/or recipient) or an organ perfusate, is screened for the presence of (e.g., detecting or identifying), pathogens, viral particles, virus-like particles (VLPs), extracellular vesicles, such as exosomes or ectosomes, miRNAs, nucleic acids, such as DNA or RNA, circulating extracellular DNA (ceDNA), Neutrophil extracellular traps (NETs) and their components including DNA, extracellular vesicle associated DNA, histones, neutrophil granule proteins and/or oxidant enzymes, inflammatory or immunomodulatory glycoproteins, glycolipids or lipoglycans or fragments thereof, before, during, or after the lectin and/or antibody affinity isolation or capture. In some embodiments, the glycan profile of extracellular vesicles, such as exosomes, ectosomes, or fragments thereof, from the sample from said donor and/or recipient and/or organ, cell population, or tissue selected for transplantation is analyzed. In some embodiments, the ability of extracellular vesicles, such as exosomes, ectosomes, or fragments thereof to bind to a lectin, such as GNA, or an antibody is determined. In some embodiments, performing the method for removing pathogens, viral particles, virus-like particles (VLPs), extracellular vesicles, such as exosomes or ectosomes, miRNAs, nucleic acids, such as DNA or RNA, circulating extracellular DNA (ceDNA), Neutrophil extracellular traps (NETs) and their components including DNA, extracellular vesicle associated DNA, histones, neutrophil granule proteins and/or oxidant enzymes, inflammatory or immunomodulatory glycoproteins, glycolipids or lipoglycans, or fragments thereof accompanying an organ, cell, or tissue transplantation procedure, improves, enhances, or benefits the organ, cell population, or tissue viability, engraftment, and/or survival and/or the survival of the recipient is increased or improved, as compared to the organ, cell population, or tissue viability, engraftment, and/or survival of the transplant or recipient after transplantation in the absence of performing such methods for removing pathogens, viral particles, virus-like particles (VLPs), extracellular vesicles, such as exosomes or ectosomes, miRNAs, nucleic acids, such as DNA or RNA, circulating extracellular DNA (ceDNA), Neutrophil extracellular traps (NETs) and their components including DNA, extracellular vesicle associated DNA, histones, neutrophil granule proteins and/or oxidant enzymes inflammatory or immunomodulatory glycoproteins, glycolipids or lipoglycans, or fragments thereof. In some embodiments, performing one or more of the methods disclosed herein for removing pathogens, viral particles, virus-like particles (VLPs), extracellular vesicles, such as exosomes or ectosomes, miRNAs, nucleic acids, such as DNA or RNA, circulating extracellular DNA (ceDNA), Neutrophil extracellular traps (NETs) and their components including DNA, extracellular vesicle associated DNA, histones, neutrophil granule proteins and/or oxidant enzymes, inflammatory or immunomodulatory glycoproteins, glycolipids or lipoglycans, or fragments thereof on an organ perfusate obtained from an organ prior to transplantation removes or reduces the presence of molecules or compositions from the organ perfusate and organ, which are associated with tissue rejection, organ failure, necrosis, or poor patient outcome after transplantation, as compared to the organ perfusate and organ prior to performing the one or more removal methods described herein.

Preferred aspects of the present invention are related to the following numbered alternatives:
1. A method for removing a pathogen, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof in a subject, wherein said subject is an organ, cell, or tissue donor, the method comprising:
   a) introducing a sample from said subject prior to removal of an organ, population of cells, or tissue into a vessel, such as an extracorporeal device, comprising a support, such as diatomaceous earth or an ion exchange resin, a lectin, such as Galanthus nivalis lectin (GNA), and/or an antibody, preferably, wherein the lectin or antibody or both are immobilized on the support;
   b) contacting the sample with the support, lectin and/or antibody preferably, the support in the vessel, such as in an extracorporeal device;
   c) reintroducing the sample obtained after b) into said subject, wherein the sample obtained after b) has a reduced amount of the pathogen, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicles associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof, as compared to the sample of said subject prior to b); and
   d) optionally, selecting said subject for receiving an extracorporeal therapy accompanying organ, cell, or tissue transplantation, which removes the pathogen, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof prior to (a) and/or
   e) optionally, screening for the presence of, e.g., detecting or identifying, the pathogen, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof, in a sample from said subject, such as blood or plasma, prior to (a) or after (b) or both accompanying organ, cell, or tissue transplantation; and/or
   f) optionally, analyzing the glycan profile of extracellular vesicles, such as exosomes, ectosomes, or fragments thereof, from a sample from said subject prior to a) or after b) or both e.g., determining the ability of said extracellular vesicles, such as exosomes, ectosomes, or fragments thereof to bind to said lectin, such as GNA, or said antibody or said support or any combination thereof.
2. A method for removing a pathogen, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof in a subject, wherein said subject is an organ, cell, or tissue transplant recipient, the method comprising:
   a) introducing a sample from said subject prior to or after organ, cell, or tissue transplantation into a vessel, such as an extracorporeal device, comprising a support, such as diatomaceous earth or an ion exchange resin, a lectin, such as Galanthus nivalis lectin (GNA), and/or an antibody, preferably, wherein the lectin or antibody or both are immobilized on the support;
   b) contacting the sample with the support, lectin and/or antibody and, preferably, the support in the vessel, such as in an extracorporeal device;
   c) reintroducing the sample obtained after b) into said subject, wherein the sample obtained after b) has a reduced amount of the pathogen, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof, as compared to the sample of said subject prior to b); and
   d) optionally, selecting said subject for receiving an extracorporeal therapy accompanying organ, cell, or tissue transplantation, which viral particles, virus-like particles (VLPs), extracellular vesicles, such as exosomes or ectosomes, miRNAs, a biomarker or mediator for tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof prior to (a) and/or
   e) optionally, screening for the presence of, e.g., detecting or identifying, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof, in a sample from said subject, such as blood or plasma, prior to (a) or after (b) or both accompanying organ, cell, or tissue transplantation; and/or
   f) optionally, analyzing the glycan profile of extracellular vesicles, such as exosomes, ectosomes, or fragments thereof, from a sample from said subject prior to a) or after b) or both e.g., determining the ability of said extracellular vesicles, such as exosomes, ectosomes, or fragments thereof to bind to said lectin, such as GNA, or said antibody or said support or any combination thereof.
3. A method for removing a pathogen, viral particles, virus-like particles (VLPs), extracellular vesicles, such as exosomes or ectosomes, miRNAs, a biomarker of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof from an organ, organ perfusate, population of cells, or tissue, the method comprising:
   a) introducing a sample from said organ, organ perfusate, population of cells, or tissue, preferably, a perfused organ or organ perfusate, isolated from a donor, preferably a deceased donor e.g., a donation after circulatory death (DCD) or a donation after brain death (DBD), into a vessel, such as an extracorporeal device, comprising a support, such as diatomaceous earth or an ion exchange resin, a lectin, such as Galanthus nivalis lectin (GNA), and/or an antibody, preferably, wherein the lectin or antibody or both are immobilized on the support;
   b) contacting the sample with the support, lectin and/or antibody and, preferably, the support in the vessel, such as in an extracorporeal device;
   c) reintroducing the sample obtained after b) into said organ, population of cells, or tissue, wherein the sample obtained after b) has a reduced amount of the pathogen, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof, as compared to the sample prior to b); and
   d) optionally, selecting said organ, organ perfusate, population of cells, or tissue for receiving an extracorporeal therapy, which removes the pathogen, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof prior to (a) and/or
   e) optionally, screening for the presence of, e.g., detecting or identifying, the pathogen, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof, in a sample from said organ, such as blood, plasma or organ perfusion fluid, prior to (a) or after (b) or both accompanying organ, cell, or tissue transplantation; and/or
   f) optionally, analyzing the glycan profile of extracellular vesicles, such as exosomes, ectosomes, or fragments thereof, from a sample from said organ or organ perfusate, prior to a) or after b) or both e.g., determining the ability of said extracellular vesicles, such as exosomes, ectosomes, or fragments thereof to bind to said lectin, such as GNA, or said antibody or said support or any combination thereof.
4. A method of organ, cell, or tissue transplantation comprising performing the method of any one of alternatives 1-3 accompanying an organ, cell, or tissue transplantation procedure, preferably wherein organ, cell, or tissue viability, such as tissue functionality including, e.g., production of urine when the organ is a kidney, engraftment, and/or survival of the transplant and/or recipient is increased or improved as compared to the organ, cell, or tissue viability, engraftment, and/or survival of the transplant or recipient after transplantation in the absence of the methods of any one of alternatives 1-3 or, wherein the presence of molecules or compositions from the organ perfusate and/or organ, which are associated with tissue rejection, organ failure, necrosis, or poor patient outcome after transplantation, are reduced as compared to the organ perfusate and/or organ prior to performing the method of any one of alternatives 1-3.
5. The method of any one of alternatives 1-4, further comprising repeating steps b) and c) to further reduce the amount of the pathogen, viral particles, virus-like particles (VLPs), extracellular vesicles, such as exosomes or ectosomes, mi RNAs, a biomarker of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof in the sample.
6. The method of any one of alternatives 1-5, wherein the viral particles or fragments thereof are from Human immunodeficiency virus (HIV), Hepatitis C virus (HCV), Hepatitis B (HBV), Hepatitis D virus (HDV); herpes family viruses such as HTLV-1, HSV1/2, varicella zoster virus (VZV), Human herpesvirus (HHV)-6, HHV-7, HHV-8, EBV, Kaposi's sarcoma-associated herpesvirus (KSHV), cytomegalovirus (CMV); Human papilloma virus (HPV), picornaviruses including coronaviruses such as SARS-CoV2, SARS-CoV2 Spike protein and variants thereof, SARS, MERS, OC43, 229E, NL63, HKU1 porcine epidemic diarrhea virus (PEDV), Transmissible gastroenteritis virus (TGEV), enteroviruses, such as EV-D68, A71, Coxsackie A/B, echoviruses, polioviruses; rhinoviruses, noroviruses, porcine cytomegalovirus (pCMV), porcine reseolovirus, porcine Retroviruses, porcine endogeneous retroviruses (PERVs) A, B, C, porcine lymphotrophic herpesviruses (pLHV), or porcine circovirus or any combination thereof.
7. The method of any one of alternatives 1-6, wherein the lectin binds to a viral coat or envelope protein or a fragment thereof.
8. The method of any one of alternatives 1-7, wherein the pathogen is Mycoplasma or Ureaplasma, such as M. amphoriforme, M. buccale, M. faucium, M. fermentans, M. genitalium, M. hominis, M. incognitus, M. lipophilum, M. orale, M. penetrans, M. pirum, M. pneumoniae, M. primatum, M. salivarium, M. spermatophilum, or Ureaplasma urealyticum.
9. The method of any of alternatives 1-8, wherein the miRNAs comprise miR23a, miR 374b-5p, miR28-5p and/or miR24-3p.
10. The method of any one of alternatives 1-9, wherein inflammatory or immunomodulatory glycoproteins, glycolipids or lipoglycans, or fragments thereof are removed.
11. The method of any one of alternatives 1-10, wherein the miRNAs comprise any one or more of: hsa-let-7a-5p, hsa-let-7b-5p, hsa-let-7c-5p, hsa-let-7d-5p, hsa-miR-29b-3p, hsa-miR-99a-5p, hsa-miR-15b-5p, hsa-miR-1-3p, hsa-miR-548aa, hsa-miR-106a-5p, hsa-miR-106b-5p, hsa-miR-10a-5p, hsa-miR-10b-5p, hsa-miR-1180-3p, hsa-miR-1183, hsa-miR-122-5p, hsa-miR-1246, hsa-miR-1253, hsa-miR-125a-5p, hsa-miR-125b-5p, hsa-miR-100-5p, hsa-miR-548ah-5p, hsa-miR-98-5p, hsa-miR-4454, hsa-miR-128-3p, hsa-miR-1285-5p, hsa-miR-1268a, hsa-miR-1299, hsa-miR-1306-5p, hsa-miR-1290, hsa-miR-15a-5p, hsa-miR-132-3p, hsa-miR-323a-3p, hsa-miR-135a-5p, hsa-miR-135b-5p, hsa-miR-140-3p, hsa-miR-140-5p, hsa-miR-141-3p, hsa-miR-193a-5p, hsa-miR-143-3p, hsa-miR-144-3p, hsa-miR-126-3p, hsa-miR-199b-5p, hsa-miR-142-3p, hsa-miR-424-5p, hsa-miR-150-5p, hsa-miR-151a-3p, hsa-miR-152-3p, hsa-miR-1537-3p, hsa-miR-154-5p, hsa-miR-1972, hsa-miR-361-5p, hsa-miR-24-3p, hsa-miR-16-5p, hsa-miR-181a-2-3p, hsa-miR-181a-5p, hsa-miR-181c-5p, hsa-miR-1827, hsa-miR-186-5p, hsa-miR-187-3p, hsa-miR-4455, hsa-miR-190b, hsa-miR-191-5p, hsa-miR-1910-5p, hsa-miR-1915-3p, hsa-miR-193a-3p, hsa-miR-23b-3p, hsa-miR-193b-3p, hsa-miR-194-5p, hsa-miR-195-5p, hsa-miR-505-3p, hsa-miR-574-3p, hsa-miR-1976, hsa-miR-199a-3p, hsa-miR-199a-5p, hsa-miR-30e-3p, hsa-miR-19a-3p, hsa-miR-19b-3p, hsa-miR-200a-3p, hsa-miR-200b-3p, hsa-miR-203a-5p, hsa-miR-204-5p, hsa-miR-205-5p, hsa-miR-20a-5p, hsa-miR-21-5p, hsa-miR-548ar-5p, hsa-miR-214-3p, hsa-miR-146a-5p, hsa-miR-335-5p, hsa-miR-22-3p, hsa-miR-221-3p, hsa-miR-222-3p, hsa-miR-223-3p, hsa-miR-660-5p, hsa-miR-377-3p, hsa-miR-145-5p, hsa-miR-218-5p, hsa-miR-26a-5p, hsa-miR-26b-5p, hsa-miR-27a-3p, hsa-miR-27b-3p, hsa-miR-28-3p, hsa-miR-28-5p, hsa-miR-299-3p, hsa-miR-29a-3p, hsa-miR-495-3p, hsa-miR-29c-3p, hsa-miR-301a-3p, hsa-miR-302b-3p, hsa-miR-30a-3p, hsa-let-7g-5p, hsa-miR-30b-5p, hsa-miR-30c-5p, hsa-miR-127-3p, hsa-miR-4286, hsa-miR-30e-5p, hsa-miR-3158-3p, hsa-miR-3168, hsa-miR-3180-5p, hsa-miR-3195, hsa-miR-32-5p, hsa-miR-320a, hsa-miR-320e, hsa-let-7e-5p, hsa-miR-323b-3p, hsa-miR-324-5p, hsa-miR-148a-3p, hsa-miR-337-3p, hsa-miR-337-5p, hsa-miR-33a-5p, hsa-miR-340-5p, hsa-miR-342-3p, hsa-miR-34a-5p, hsa-miR-361-3p, hsa-miR-497-5p, hsa-miR-362-3p, hsa-miR-363-3p, hsa-miR-365a-3p, hsa-miR-664a-3p, hsa-miR-374a-5p, hsa-miR-376a-3p, hsa-miR-376c-3p, hsa-miR-378d, hsa-miR-378f, hsa-miR-378g, hsa-miR-378i, hsa-miR-379-5p, hsa-miR-382-5p, hsa-miR-423-3p, hsa-miR-423-5p, hsa-miR-130a-3p, hsa-miR-425-5p, hsa-miR-190a-5p, hsa-miR-2117, hsa-miR-548z, hsa-miR-25-3p, hsa-miR-1323, hsa-miR-4516, hsa-miR-451a, hsa-miR-487a-3p, hsa-miR-4532, hsa-miR-455-5p, hsa-miR-483-5p, hsa-miR-486-3p, hsa-miR-4443, hsa-miR-487b-3p, hsa-miR-491-5p, hsa-miR-543, hsa-miR-496, hsa-let-7f-5p, hsa-miR-499a-5p, hsa-miR-503-5p, hsa-miR-148b-3p, hsa-miR-509-3p, hsa-miR-509-5p, hsa-miR-513b-5p, hsa-miR-513c-5p, hsa-miR-514a-3p, hsa-miR-514a-5p, hsa-miR-514b-5p, hsa-miR-518b, hsa-miR-5196-3p, hsa-miR-526a, hsa-miR-548d-5p, hsa-miR-30a-5p, hsa-miR-30d-5p, hsa-miR-196a-5p, hsa-miR-136-5p, hsa-miR-4421, hsa-miR-548m, hsa-miR-548n, hsa-miR-99b-5p, hsa-miR-551b-3p, hsa-miR-556-3p, hsa-miR-23a-3p, hsa-miR-574-5p, hsa-miR-575, hsa-miR-578, hsa-miR-584-3p, hsa-miR-655-3p, hsa-miR-590-5p, hsa-miR-593-3p, hsa-miR-597-5p, hsa-miR-601, hsa-miR-607, hsa-miR-616-3p, hsa-miR-630, hsa-miR-663a, hsa-miR-7-5p, hsa-miR-764, hsa-miR-769-5p, hsa-miR-887-3p, hsa-miR-888-5p, hsa-miR-93-5p and/or hsa-miR-95-3p.
12. The method of any one of alternatives 1-11, wherein the miRNAs comprise any one or more of: hsa-let-7a-5p, hsa-miR-29b-3p, hsa-miR-99a-5p, hsa-miR-15b-5p, hsa-miR-548aa, hsa-miR-15a-5p, hsa-miR-193a-5p, hsa-miR-126-3p, hsa-miR-1972 and/or hsa-miR-196a-5p.
13. The method of any one of alternatives 1-12, wherein the miRNAs comprise any one or more of hsa-let-7a-5p, hsa-miR-142-3p, hsa-miR-29b-3p and/or hsa-miR-99a-5p.
14. The method of any one of alternatives 1-13, wherein the biomarker or mediator for tissue damage comprise Complement C2, Complement C4b, Complement C5, Mannose Binding Lectin, Complement C1q, BDNF, Cathepsin D, NCAM, PAI-1 (Total), PDGF-AA, PDGF-AB/BB, RANTES, sICAM-1, sVCAM-1, FGF-2, Fractalkine, GROα, IFNα2, IL-1RA, IL-2, IL-3, IL-4, IL-8, IL-9, IL-10, IL-12p40, IL-13, IL-15, IP-10, MCP-1, MCP-3, MDC, MIP-1β, sCD40L, TNFα, TNFβ, 6CKine, APRIL, BCA-1, CCL28, CTACK, ENA-78, Eotaxin-2, Eotaxin-3, GCP-2, Granzyme A, Granzyme B, HMGB1, I-TAC, IFNβ, IFNω, IL-11, IL-16, IL-20, IL-24, IL-31, IL-33, IL-34, IL-35, LIF, MCP-2, MCP-4, MIP 1δ, MIP-3α, MIP-3β, MPIF-1, Perforin, sCD137, SDF-1, sFas, sFasL, TARC, TPO and/or TRAIL.
15. The method of any one of alternatives 1-14, wherein the biomarker or mediator of tissue damage comprises any one or more of: Complement C2, Complement C4b, Complement C1q, BDNF, PDGF-AB/BB, RANTES, sVCAM-1, Fractalkine, IFNα2, IL-9, MIP-1β, 6CKine, APRIL, BCA-1, CTACK, ENA-78, Eotaxin-3, GCP-2, IFNβ, IL-16, IL-20, IL-31, IL-33, IL-34, MCP-2, MIP-3α, MIP-3β, Perforin, SDF-1, TARC and/or TPO.
16. The method of any one of alternatives 1-15, wherein the biomarker or mediator of tissue damage comprises any one or more of BDNF, C1QBP, C2, C4B, C5, CCL13, CCL15, CCL17, CCL19, CCL20, CCL22, CCL23, CCL26, CCL27, CCL3, CCL4, CCL5-2, CCL5, CCL8, CTSD, CX3CL1, CXCL10, CXCL11, CXCL12, CXCL13, CXCL5, FASLG, FGF2, GZMA, GZMB, HMGB1, ICAM1, IFNA2, IL16, IL1RN, IL20, IL33, IL34, IL9, LMAN1, NCAM, PDGFA, PDGFB, PRF1, SERPINE1, TNFRSF9, TNFSF13, TNFSF13B, TPO, TUBGCP2 and/or VCAM1.
17. The method of any one of alternatives 1-16, wherein the pathogen, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof are removed from the organ, cell, or tissue donor before the organ, cell, or tissue is harvested from the donor.
18. The method of any one of alternatives 1-17, wherein the pathogen, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof are removed from the organ, organ perfusate, population of cells, or tissue after the organ, population of cells, or tissue is harvested from the donor and before the organ, population of cells, or tissue is transplanted into the recipient.
19. The method of any one of alternatives 1-18, wherein the pathogen, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof are removed from the organ, organ perfusate, population of cells, or tissue after the organ, population of cells, or tissue is harvested from the donor and prior to transplantation into the recipient.
20. The method of any one of alternatives 1-19, wherein the organ is a solid organ or the organ perfusate is from a solid organ.
21. The method of alternative 20, wherein the solid organ is selected from kidney, liver, intestines, heart, lung, or pancreas.
22. The method of any one of alternatives 1-21, wherein the population of cells are chimeric antigen receptor T cells (CAR T cells), immune cells, bone marrow cells, stem cells, or pancreatic islet cells.
23. The method of any one of alternatives 1-22, wherein the lectin immobilized on the support is selected from the group consisting of Galanthus nivalis lectin (GNA), Narcissus pseudonarcissus lectin (NPA), Allium sativum lectin (ASA), Lens culinaris lectin (LCH), Sambucus nigra lectin (SNA), Maackia amurensis lectin (MAL), and concanavalin A.
24. The method of any one of alternatives 1-23, wherein the sample is selected from peripheral blood, serum, plasma, organ perfusion fluid or organ perfusate, such as near normothermic organ perfusion fluid or near normothermic organ perfusate or hypothermic organ perfusion fluid or hypothermic organ perfusate, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, umbilical cord blood, ascites fluid, cell culture, such as CAR T cell culture, stem cell culture, or pancreatic islet culture.
25. The method of alternative 24, wherein the biological sample is mammalian, such as human or porcine.
26. The method of alternative 25, wherein the biological sample is human.
27. The method of any one of alternatives 1-26, wherein the support comprises a material selected from the group consisting of Sepharose, agarose, latex, glass, thermoplastic polymers, polystyrene, polyethylene beads, polyvinyl, diatomaceous earth, nitrocellulose and silicon.
28. The method of any one of alternatives 1-27, wherein the vessel is a plate, such as a multi-well plate, a column, a cartridge, a tube, a flask, or a beaker.
29. Use of a support, such as diatomaceous earth or an ion exchange resin, a lectin, such as Galanthus nivalis lectin (GNA), or an antibody, preferably, wherein the lectin or antibody or both are immobilized on the support to improve, enhance, or increase organ, cell, or tissue viability, engraftment, and/or survival of a transplant or to remove viral particles, virus-like particles (VLPs), extracellular vesicles, such as exosomes or ectosomes, miRNAs, a biomarker or mediator for tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof from an organ, organ perfusate, cell population, or tissue ex vivo, as compared to organ, cell, or tissue viability, engraftment, and/or survival of the transplant or the organ perfusate in the absence of said lectin and/or antibody affinity isolation or capture.
30. The use of alternative 29, wherein said organ is a solid organ, preferably selected from kidney, liver, intestines, heart, lung, or pancreas or, wherein the cell population is selected from chimeric antigen receptor T cells (CAR T cells), bone marrow cells, stem cells, or pancreatic islet cells.
31. A device for use in removing a pathogen, viral particles, virus-like particles (VLPs), extracellular vesicles, such as exosomes or ectosomes, miRNAs, a biomarker or mediator for tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof from an organ, organ perfusate, cell population, or tissue ex vivo, wherein said device comprises a support, such as diatomaceous earth or an ion exchange resin, and optionally a lectin, such as Galanthus nivalis lectin (GNA), or an antibody, preferably, wherein the lectin or antibody or both are immobilized on the support.
32. The device for use as set forth in alternative 31, wherein the viral particles or fragments thereof are from Human immunodeficiency virus (HIV), Hepatitis C virus (HCV), Hepatitis B (HBV), Hepatitis D virus (HDV); herpes family viruses such as HTLV-1, HSV1/2, varicella zoster virus (VZV), Human herpesvirus (HHV)-6, HHV-7, HHV-8, EBV, Kaposi's sarcoma-associated herpesvirus (KSHV), cytomegalovirus (CMV); Human papilloma virus (HPV), picornaviruses including coronaviruses such as SARS-CoV2, SARS-CoV2 Spike protein and variants thereof, SARS, MERS, OC43, 229E, NL63, HKU1 porcine epidemic diarrhea virus (PEDV), Transmissible gastroenteritis virus (TGEV), enteroviruses, such as EV-D68, A71, Coxsackie A/B, echoviruses, polioviruses; rhinoviruses, noroviruses, porcine cytomegalovirus (pCMV), porcine reseolovirus, porcine Retroviruses, porcine endogeneous retroviruses (PERVs) A, B, C, porcine lymphotrophic herpesviruses (pLHV), or porcine circovirus or any combination thereof.
33. The device for use as set forth in alternative 31 or 32, wherein the lectin binds to a viral coat or envelope protein or a fragment thereof.
34. The device for use as set forth in any one of alternatives 31-33, wherein the pathogen is Mycoplasma or Ureaplasma, such as M. amphoriforme, M. buccale, M. faucium, M. fermentans, M. genitalium, M. hominis, M. incognitus, M. lipophilum, M. orale, M. penetrans, M. pirum, M. pneumoniae, M. primatum, M. salivarium, M. spermatophilum, or Ureaplasma urealyticum.
35. The device for use as set forth in any one of alternatives 31-34, wherein the miRNA comprises any one or more of: hsa-let-7a-5p, hsa-miR-29b-3p, hsa-miR-99a-5p, hsa-miR-15b-5p, hsa-miR-548aa, hsa-miR-15a-5p, hsa-miR-193a-5p, hsa-miR-126-3p, hsa-miR-1972 and/or hsa-miR-196a-5p.
36. The device for use as set forth in any one of alternatives 31-35, wherein the miRNAs comprise any one or more of: hsa-let-7a-5p, hsa-let-7b-5p, hsa-let-7c-5p, hsa-let-7d-5p, hsa-miR-29b-3p, hsa-miR-99a-5p, hsa-miR-15b-5p, hsa-miR-1-3p, hsa-miR-548aa, hsa-miR-106a-5p, hsa-miR-106b-5p, hsa-miR-10a-5p, hsa-miR-10b-5p, hsa-miR-1180-3p, hsa-miR-1183, hsa-miR-122-5p, hsa-miR-1246, hsa-miR-1253, hsa-miR-125a-5p, hsa-miR-125b-5p, hsa-miR-100-5p, hsa-miR-548ah-5p, hsa-miR-98-5p, hsa-miR-4454, hsa-miR-128-3p, hsa-miR-1285-5p, hsa-miR-1268a, hsa-miR-1299, hsa-miR-1306-5p, hsa-miR-1290, hsa-miR-15a-5p, hsa-miR-132-3p, hsa-miR-323a-3p, hsa-miR-135a-5p, hsa-miR-135b-5p, hsa-miR-140-3p, hsa-miR-140-5p, hsa-miR-141-3p, hsa-miR-193a-5p, hsa-miR-143-3p, hsa-miR-144-3p, hsa-miR-126-3p, hsa-miR-199b-5p, hsa-miR-142-3p, hsa-miR-424-5p, hsa-miR-150-5p, hsa-miR-151a-3p, hsa-miR-152-3p, hsa-miR-1537-3p, hsa-miR-154-5p, hsa-miR-1972, hsa-miR-361-5p, hsa-miR-24-3p, hsa-miR-16-5p, hsa-miR-181a-2-3p, hsa-miR-181a-5p, hsa-miR-181c-5p, hsa-miR-1827, hsa-miR-186-5p, hsa-miR-187-3p, hsa-miR-4455, hsa-miR-190b, hsa-miR-191-5p, hsa-miR-1910-5p, hsa-miR-1915-3p, hsa-miR-193a-3p, hsa-miR-23b-3p, hsa-miR-193b-3p, hsa-miR-194-5p, hsa-miR-195-5p, hsa-miR-505-3p, hsa-miR-574-3p, hsa-miR-1976, hsa-miR-199a-3p, hsa-miR-199a-5p, hsa-miR-30e-3p, hsa-miR-19a-3p, hsa-miR-19b-3p, hsa-miR-200a-3p, hsa-miR-200b-3p, hsa-miR-203a-5p, hsa-miR-204-5p, hsa-miR-205-5p, hsa-miR-20a-5p, hsa-miR-21-5p, hsa-miR-548ar-5p, hsa-miR-214-3p, hsa-miR-146a-5p, hsa-miR-335-5p, hsa-miR-22-3p, hsa-miR-221-3p, hsa-miR-222-3p, hsa-miR-223-3p, hsa-miR-660-5p, hsa-miR-377-3p, hsa-miR-145-5p, hsa-miR-218-5p, hsa-miR-26a-5p, hsa-miR-26b-5p, hsa-miR-27a-3p, hsa-miR-27b-3p, hsa-miR-28-3p, hsa-miR-28-5p, hsa-miR-299-3p, hsa-miR-29a-3p, hsa-miR-495-3p, hsa-miR-29c-3p, hsa-miR-301a-3p, hsa-miR-302b-3p, hsa-miR-30a-3p, hsa-let-7g-5p, hsa-miR-30b-5p, hsa-miR-30c-5p, hsa-miR-127-3p, hsa-miR-4286, hsa-miR-30e-5p, hsa-miR-3158-3p, hsa-miR-3168, hsa-miR-3180-5p, hsa-miR-3195, hsa-miR-32-5p, hsa-miR-320a, hsa-miR-320e, hsa-let-7e-5p, hsa-miR-323b-3p, hsa-miR-324-5p, hsa-miR-148a-3p, hsa-miR-337-3p, hsa-miR-337-5p, hsa-miR-33a-5p, hsa-miR-340-5p, hsa-miR-342-3p, hsa-miR-34a-5p, hsa-miR-361-3p, hsa-miR-497-5p, hsa-miR-362-3p, hsa-miR-363-3p, hsa-miR-365a-3p, hsa-miR-664a-3p, hsa-miR-374a-5p, hsa-miR-376a-3p, hsa-miR-376c-3p, hsa-miR-378d, hsa-miR-378f, hsa-miR-378g, hsa-miR-378i, hsa-miR-379-5p, hsa-miR-382-5p, hsa-miR-423-3p, hsa-miR-423-5p, hsa-miR-130a-3p, hsa-miR-425-5p, hsa-miR-190a-5p, hsa-miR-2117, hsa-miR-548z, hsa-miR-25-3p, hsa-miR-1323, hsa-miR-4516, hsa-miR-451a, hsa-miR-487a-3p, hsa-miR-4532, hsa-miR-455-5p, hsa-miR-483-5p, hsa-miR-486-3p, hsa-miR-4443, hsa-miR-487b-3p, hsa-miR-491-5p, hsa-miR-543, hsa-miR-496, hsa-let-7f-5p, hsa-miR-499a-5p, hsa-miR-503-5p, hsa-miR-148b-3p, hsa-miR-509-3p, hsa-miR-509-5p, hsa-miR-513b-5p, hsa-miR-513c-5p, hsa-miR-514a-3p, hsa-miR-514a-5p, hsa-miR-514b-5p, hsa-miR-518b, hsa-miR-5196-3p, hsa-miR-526a, hsa-miR-548d-5p, hsa-miR-30a-5p, hsa-miR-30d-5p, hsa-miR-196a-5p, hsa-miR-136-5p, hsa-miR-4421, hsa-miR-548m, hsa-miR-548n, hsa-miR-99b-5p, hsa-miR-551b-3p, hsa-miR-556-3p, hsa-miR-23a-3p, hsa-miR-574-5p, hsa-miR-575, hsa-miR-578, hsa-miR-584-3p, hsa-miR-655-3p, hsa-miR-590-5p, hsa-miR-593-3p, hsa-miR-597-5p, hsa-miR-601, hsa-miR-607, hsa-miR-616-3p, hsa-miR-630, hsa-miR-663a, hsa-miR-7-5p, hsa-miR-764, hsa-miR-769-5p, hsa-miR-887-3p, hsa-miR-888-5p, hsa-miR-93-5p and/or hsa-miR-95-3p.
37. The device for use as set forth in any one of alternatives 31-36, wherein the miRNAs comprise any one or more of: hsa-miR1285-5p, hsa-miR-575, hsa-miR-223-3p, hsa-miR-127-3p, hsa-miR-29b-3p, hsa-miR-3144-3p, hsa-miR-28-5p and/or hsa-miR-1286.
38. The device for use as set forth in any one of alternatives 31-37, wherein the miRNAs comprise any one or more of: hsa-let-7a-5p, hsa-miR-142-3p, hsa-miR-29b-3p and/or hsa-miR-99a-5p.
39. The device for use as set forth in any one of alternatives 31-38, wherein the biomarker or mediator of tissue damage comprise any one or more of: Complement C2, Complement C4b, Complement C5, Mannose Binding Lectin, Complement C1q, BDNF, Cathepsin D, NCAM, PAI-1 (Total), PDGF-AA, PDGF-AB/BB, RANTES, sICAM-1, sVCAM-1, FGF-2, Fractalkine, GROα, IFNα2, IL-1RA, IL-2, IL-3, IL-4, IL-8, IL-9, IL-10, IL-12p40, IL-13, IL-15, IP-10, MCP-1, MCP-3, MDC, MIP-1β, sCD40L, TNFα, TNFβ, 6CKine, APRIL, BCA-1, CCL28, CTACK, ENA-78, Eotaxin-2, Eotaxin-3, GCP-2, Granzyme A, Granzyme B, HMGB1, I-TAC, IFNβ, IFNω, IL-11, IL-16, IL-20, IL-24, IL-31, IL-33, IL-34, IL-35, LIF, MCP-2, MCP-4, MIP 1δ, MIP-3α, MIP-3β, MPIF-1, Perforin, sCD137, SDF-1, sFas, sFasL, TARC, TPO, and/or TRAIL.
40. The device for use as set forth in any one of alternatives 31-39, wherein the biomarker or mediator of tissue damage comprises any one or more of: Complement C2, Complement C4b, Complement C1q, BDNF, PDGF-AB/BB, RANTES, sVCAM-1, Fractalkine, IFNα2, IL-9, MIP-1β, 6CKine, APRIL, BCA-1, CTACK, ENA-78, Eotaxin-3, GCP-2, IFNβ, IL-16, IL-20, IL-31, IL-33, IL-34, MCP-2, MIP-3α, MIP-3β, Perforin, SDF-1, TARC and/or TPO.
41. The device for use as set forth in any one of alternatives 31-40, wherein the biomarker or mediator of tissue damage comprises any one or more of: BDNF, C1QBP, C2, C4B, C5, CCL13, CCL15, CCL17, CCL19, CCL20, CCL22, CCL23, CCL26, CCL27, CCL3, CCL4, CCL5-2, CCL5, CCL8, CTSD, CX3CL1, CXCL10, CXCL11, CXCL12, CXCL13, CXCL5, FASLG, FGF2, GZMA, GZMB, HMGB1, ICAM1, IFNA2, IL16, IL1RN, IL20, IL33, IL34, IL9, LMAN1, NCAM, PDGFA, PDGFB, PRF1, SERPINE1, TNFRSF9, TNFSF13, TNFSF13B, TPO, TUBGCP2 and/or VCAM1.
42. The device for use as set forth in any one of alternatives 31-41, wherein inflammatory or immunomodulatory glycoproteins, glycolipids or lipoglycans, or fragments thereof are removed.
43. The device for use as set forth in any one of alternatives 31-42, wherein the organ is a solid organ or the organ perfusate is from a solid organ.
44. The device for use as set forth in alternative 43, wherein the solid organ is selected from kidney, liver, intestines, heart, lung, or pancreas.
45. The device for use as set forth in any one of alternatives 31-44, wherein the population of cells are chimeric antigen receptor T cells (CAR T cells), immune cells, bone marrow cells, stem cells, or pancreatic islet cells.
46. The device for use as set forth in any one of alternatives 31-45, wherein the lectin immobilized on the support is selected from the group consisting of Galanthus nivalis lectin (GNA), Narcissus pseudonarcissus lectin (NPA), Allium sativum lectin (ASA), Lens culinaris lectin (LCH), Sambucus nigra lectin (SNA), Maackia amurensis lectin (MAL), and concanavalin A.
47. The device for use as set forth in any one of alternatives 31-46, wherein the support comprises a material selected from the group consisting of Sepharose, agarose, latex, glass, thermoplastic polymers, polystyrene, polyethylene beads, polyvinyl, diatomaceous earth, nitrocellulose and silicon.
48. The device for use as set forth in any one of alternatives 31-47, wherein the vessel is a plate, such as a multi-well plate, a column, a cartridge, a tube, a flask, or a beaker.
49. A device comprising a support, such as diatomaceous earth or an ion exchange resin, and optionally a lectin, such as Galanthus nivalis lectin (GNA), or an antibody, preferably, wherein the lectin or antibody or both are immobilized on the support, wherein the support, lectin and/or antibody is joined to a pathogen, viral particles, virus-like particles (VLPs), extracellular vesicles, such as exosomes or ectosomes, miRNAs, a biomarker or mediator for tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof from an organ, organ perfusate, cell population, or tissue
50. The device as set forth in alternative 49, wherein the viral particles or fragments thereof are from Human immunodeficiency virus (HIV), Hepatitis C virus (HCV), Hepatitis B (HBV), Hepatitis D virus (HDV); herpes family viruses such as HTLV-1, HSV1/2, varicella zoster virus (VZV), Human herpesvirus (HHV)-6, HHV-7, HHV-8, EBV, Kaposi's sarcoma-associated herpesvirus (KSHV), cytomegalovirus (CMV); Human papilloma virus (HPV), picornaviruses including coronaviruses such as SARS-CoV2, SARS-CoV2 Spike protein and variants thereof, SARS, MERS, OC43, 229E, NL63, HKU1 porcine epidemic diarrhea virus (PEDV), Transmissible gastroenteritis virus (TGEV), enteroviruses, such as EV-D68, A71, Coxsackie A/B, echoviruses, polioviruses; rhinoviruses, noroviruses, porcine cytomegalovirus (pCMV), porcine reseolovirus, porcine Retroviruses, porcine endogeneous retroviruses (PERVs) A, B, C, porcine lymphotrophic herpesviruses (pLHV), or porcine circovirus or any combination thereof.
51. The device as set forth in alternative 49 or 50, wherein the lectin binds to a viral coat or envelope protein or a fragment thereof.
52. The device as set forth in any one of alternatives 49-51, wherein the pathogen is Mycoplasma or Ureaplasma, such as M. amphoriforme, M. buccale, M. faucium, M. fermentans, M. genitalium, M. hominis, M. incognitus, M. lipophilum, M. orale, M. penetrans, M. pirum, M. pneumoniae, M. primatum, M. salivarium, M. spermatophilum, or Ureaplasma urealyticum.
53. The device as set forth in any one of alternatives 49-52, wherein the miRNA comprises any one or more of: hsa-let-7a-5p, hsa-miR-29b-3p, hsa-miR-99a-5p, hsa-miR-15b-5p, hsa-miR-548aa, hsa-miR-15a-5p, hsa-miR-193a-5p, hsa-miR-126-3p, hsa-miR-1972 and/or hsa-miR-196a-5p.
54. The device as set forth in any one of alternatives 49-53, wherein the miRNAs comprise any one or more of: hsa-let-7a-5p, hsa-let-7b-5p, hsa-let-7c-5p, hsa-let-7d-5p, hsa-miR-29b-3p, hsa-miR-99a-5p, hsa-miR-15b-5p, hsa-miR-1-3p, hsa-miR-548aa, hsa-miR-106a-5p, hsa-miR-106b-5p, hsa-miR-10a-5p, hsa-miR-10b-5p, hsa-miR-1180-3p, hsa-miR-1183, hsa-miR-122-5p, hsa-miR-1246, hsa-miR-1253, hsa-miR-125a-5p, hsa-miR-125b-5p, hsa-miR-100-5p, hsa-miR-548ah-5p, hsa-miR-98-5p, hsa-miR-4454, hsa-miR-128-3p, hsa-miR-1285-5p, hsa-miR-1268a, hsa-miR-1299, hsa-miR-1306-5p, hsa-miR-1290, hsa-miR-15a-5p, hsa-miR-132-3p, hsa-miR-323a-3p, hsa-miR-135a-5p, hsa-miR-135b-5p, hsa-miR-140-3p, hsa-miR-140-5p, hsa-miR-141-3p, hsa-miR-193a-5p, hsa-miR-143-3p, hsa-miR-144-3p, hsa-miR-126-3p, hsa-miR-199b-5p, hsa-miR-142-3p, hsa-miR-424-5p, hsa-miR-150-5p, hsa-miR-151a-3p, hsa-miR-152-3p, hsa-miR-1537-3p, hsa-miR-154-5p, hsa-miR-1972, hsa-miR-361-5p, hsa-miR-24-3p, hsa-miR-16-5p, hsa-miR-181a-2-3p, hsa-miR-181a-5p, hsa-miR-181c-5p, hsa-miR-1827, hsa-miR-186-5p, hsa-miR-187-3p, hsa-miR-4455, hsa-miR-190b, hsa-miR-191-5p, hsa-miR-1910-5p, hsa-miR-1915-3p, hsa-miR-193a-3p, hsa-miR-23b-3p, hsa-miR-193b-3p, hsa-miR-194-5p, hsa-miR-195-5p, hsa-miR-505-3p, hsa-miR-574-3p, hsa-miR-1976, hsa-miR-199a-3p, hsa-miR-199a-5p, hsa-miR-30e-3p, hsa-miR-19a-3p, hsa-miR-19b-3p, hsa-miR-200a-3p, hsa-miR-200b-3p, hsa-miR-203a-5p, hsa-miR-204-5p, hsa-miR-205-5p, hsa-miR-20a-5p, hsa-miR-21-5p, hsa-miR-548ar-5p, hsa-miR-214-3p, hsa-miR-146a-5p, hsa-miR-335-5p, hsa-miR-22-3p, hsa-miR-221-3p, hsa-miR-222-3p, hsa-miR-223-3p, hsa-miR-660-5p, hsa-miR-377-3p, hsa-miR-145-5p, hsa-miR-218-5p, hsa-miR-26a-5p, hsa-miR-26b-5p, hsa-miR-27a-3p, hsa-miR-27b-3p, hsa-miR-28-3p, hsa-miR-28-5p, hsa-miR-299-3p, hsa-miR-29a-3p, hsa-miR-495-3p, hsa-miR-29c-3p, hsa-miR-301a-3p, hsa-miR-302b-3p, hsa-miR-30a-3p, hsa-let-7g-5p, hsa-miR-30b-5p, hsa-miR-30c-5p, hsa-miR-127-3p, hsa-miR-4286, hsa-miR-30e-5p, hsa-miR-3158-3p, hsa-miR-3168, hsa-miR-3180-5p, hsa-miR-3195, hsa-miR-32-5p, hsa-miR-320a, hsa-miR-320e, hsa-let-7e-5p, hsa-miR-323b-3p, hsa-miR-324-5p, hsa-miR-148a-3p, hsa-miR-337-3p, hsa-miR-337-5p, hsa-miR-33a-5p, hsa-miR-340-5p, hsa-miR-342-3p, hsa-miR-34a-5p, hsa-miR-361-3p, hsa-miR-497-5p, hsa-miR-362-3p, hsa-miR-363-3p, hsa-miR-365a-3p, hsa-miR-664a-3p, hsa-miR-374a-5p, hsa-miR-376a-3p, hsa-miR-376c-3p, hsa-miR-378d, hsa-miR-378f, hsa-miR-378g, hsa-miR-378i, hsa-miR-379-5p, hsa-miR-382-5p, hsa-miR-423-3p, hsa-miR-423-5p, hsa-miR-130a-3p, hsa-miR-425-5p, hsa-miR-190a-5p, hsa-miR-2117, hsa-miR-548z, hsa-miR-25-3p, hsa-miR-1323, hsa-miR-4516, hsa-miR-451a, hsa-miR-487a-3p, hsa-miR-4532, hsa-miR-455-5p, hsa-miR-483-5p, hsa-miR-486-3p, hsa-miR-4443, hsa-miR-487b-3p, hsa-miR-491-5p, hsa-miR-543, hsa-miR-496, hsa-let-7f-5p, hsa-miR-499a-5p, hsa-miR-503-5p, hsa-miR-148b-3p, hsa-miR-509-3p, hsa-miR-509-5p, hsa-miR-513b-5p, hsa-miR-513c-5p, hsa-miR-514a-3p, hsa-miR-514a-5p, hsa-miR-514b-5p, hsa-miR-518b, hsa-miR-5196-3p, hsa-miR-526a, hsa-miR-548d-5p, hsa-miR-30a-5p, hsa-miR-30d-5p, hsa-miR-196a-5p, hsa-miR-136-5p, hsa-miR-4421, hsa-miR-548m, hsa-miR-548n, hsa-miR-99b-5p, hsa-miR-551b-3p, hsa-miR-556-3p, hsa-miR-23a-3p, hsa-miR-574-5p, hsa-miR-575, hsa-miR-578, hsa-miR-584-3p, hsa-miR-655-3p, hsa-miR-590-5p, hsa-miR-593-3p, hsa-miR-597-5p, hsa-miR-601, hsa-miR-607, hsa-miR-616-3p, hsa-miR-630, hsa-miR-663a, hsa-miR-7-5p, hsa-miR-764, hsa-miR-769-5p, hsa-miR-887-3p, hsa-miR-888-5p, hsa-miR-93-5p and/or hsa-miR-95-3p.
55. The device as set forth in any one of alternatives 49-54, wherein the miRNAs comprise any one or more of: hsa-miR1285-5p, hsa-miR-575, hsa-miR-223-3p, hsa-miR-127-3p, hsa-miR-29b-3p, hsa-miR-3144-3p, hsa-miR-28-5p and/or hsa-miR-1286.
56. The device as set forth in any one of alternatives 49-55, wherein the miRNAs comprise any one or more of hsa-let-7a-5p, hsa-miR-142-3p, hsa-miR-29b-3p and/or hsa-miR-99a-5p.
57. The device as set forth in any one of alternatives 49-56, wherein the biomarker or mediator of tissue damage comprise any one or more of: Complement C2, Complement C4b, Complement C5, Mannose Binding Lectin, Complement C1q, BDNF, Cathepsin D, NCAM, PAI-1 (Total), PDGF-AA, PDGF-AB/BB, RANTES, sICAM-1, sVCAM-1, FGF-2, Fractalkine, GROα, IFNα2, IL-1RA, IL-2, IL-3, IL-4, IL-8, IL-9, IL-10, IL-12p40, IL-13, IL-15, IP-10, MCP-1, MCP-3, MDC, MIP-1β, sCD40L, TNFα, TNFβ, 6CKine, APRIL, BCA-1, CCL28, CTACK, ENA-78, Eotaxin-2, Eotaxin-3, GCP-2, Granzyme A, Granzyme B, HMGB1, I-TAC, IFNβ, IFNω, IL-11, IL-16, IL-20, IL-24, IL-31, IL-33, IL-34, IL-35, LIF, MCP-2, MCP-4, MIP 1δ, MIP-3α, MIP-3β, MPIF-1, Perforin, sCD137, SDF-1, sFas, sFasL, TARC, TPO and/or TRAIL.
58. The device as set forth in any one of alternatives 49-57, wherein the biomarker or mediator of tissue damage comprises any one or more of: Complement C2, Complement C4b, Complement C1q, BDNF, PDGF-AB/BB, RANTES, sVCAM-1, Fractalkine, IFNα2, IL-9, MIP-1β, 6CKine, APRIL, BCA-1, CTACK, ENA-78, Eotaxin-3, GCP-2, IFNβ, IL-16, IL-20, IL-31, IL-33, IL-34, MCP-2, MIP-3α, MIP-3β, Perforin, SDF-1, TARC and/or TPO.
59. The device as set forth in any one of alternatives 49-58, wherein the biomarker or mediator of tissue damage comprises any one or more of: BDNF, C1QBP, C2, C4B, C5, CCL13, CCL15, CCL17, CCL19, CCL20, CCL22, CCL23, CCL26, CCL27, CCL3, CCL4, CCL5-2, CCL5, CCL8, CTSD, CX3CL1, CXCL10, CXCL11, CXCL12, CXCL13, CXCL5, FASLG, FGF2, GZMA, GZMB, HMGB1, ICAM1, IFNA2, IL16, IL1RN, IL20, IL33, IL34, IL9, LMAN1, NCAM, PDGFA, PDGFB, PRF1, SERPINE1, TNFRSF9, TNFSFI3, TNFSF13B, TPO, TUBGCP2 and/or VCAM1.
60. The device as set forth in any one of alternatives 49-59, wherein inflammatory or immunomodulatory glycoproteins, glycolipids or lipoglycans, or fragments thereof are joined to the support, lectin and/or antibody.
61. The device as set forth in any one of alternatives 49-60, wherein the organ is a solid organ or the organ perfusate is from a solid organ.
62. The device as set forth in alternative 61, wherein the solid organ is selected from kidney, liver, intestines, heart, lung, or pancreas.
63. The device as set forth in any one of alternatives 49-62, wherein the population of cells are chimeric antigen receptor T cells (CAR T cells), immune cells, bone marrow cells, stem cells, or pancreatic islet cells.
64. The device as set forth in any one of alternatives 49-63, wherein the lectin immobilized on the support is selected from the group consisting of Galanthus nivalis lectin (GNA), Narcissus pseudonarcissus lectin (NPA), Allium sativum lectin (ASA), Lens culinaris lectin (LCH), Sambucus nigra lectin (SNA), Maackia amurensis lectin (MAL), and concanavalin A.
65. The device as set forth in any one of alternatives 49-64, wherein the support comprises a material selected from the group consisting of Sepharose, agarose, latex, glass, thermoplastic polymers, polystyrene, polyethylene beads, polyvinyl, diatomaceous earth, nitrocellulose and silicon.
66. The device as set forth in any one of alternatives 49-65, wherein the vessel is a plate, such as a multi-well plate, a column, a cartridge, a tube, a flask, or a beaker.
67. A method of preparing a kidney, which has been removed from a donor, for transplantation, preferably a kidney obtained from a deceased donor e.g., a donation after circulatory death (DCD) or a donation after brain death (DBD), the method comprising:
a) collecting a biological sample from the kidney;
b) detecting the presence or amount of at least one or more pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof;
c) comparing the detected one or more pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof to a reference level; and
d) upon determining that the detected one or more pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof are present or altered in the sample as compared to the reference level, providing a therapy to the kidney, wherein the therapy comprises:
   i. introducing perfusate from the kidney into a vessel, such as an extracorporeal device, comprising a support, such as diatomaceous earth or an ion exchange resin, a lectin, such as Galanthus nivalis lectin (GNA), and/or an antibody preferably, wherein the lectin or antibody or both are immobilized on the support;
   ii. contacting the perfusate with the support, lectin and/or antibody in the vessel; and
   iii. reintroducing the perfusate obtained after ii) into the kidney, wherein the perfusate obtained after ii) has a reduced amount of pathogens, viral particles, virus-like particles (VLPs), extracellular vesicles, such as exosomes or ectosomes, miRNAs, a biomarker or mediator for tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof, as compared to the perfusate prior to ii).
68. The method of alternative 67, wherein the presence or amount of at least one or more of the pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof is detected using Western blot, enzyme-linked immunosorbent assay (ELISA), Northern blotting, quantitative polymerase chain reaction (qPCR) protein microarray, DNA microarray, gene expression analysis or RNA-seq.
69. A method of any one of alternatives 67-68, wherein kidney viability, engraftment, and/or survival of the transplant and/or recipient is increased or improved as compared to the kidney viability, engraftment, and/or survival of the transplant or recipient after transplantation in the absence of the methods of any one of alternatives 67-68.
70. The method of any one of alternatives 67-69, further comprising repeating steps (i)-(iii) to further reduce the amount of amount of the pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof.
71. The method of any one of alternatives 67-70, wherein the miRNAs comprise any one or more of: hsa-let-7a-5p, hsa-let-7b-5p, hsa-let-7c-5p, hsa-let-7d-5p, hsa-miR-29b-3p, hsa-miR-99a-5p, hsa-miR-15b-5p, hsa-miR-1-3p, hsa-miR-548aa, hsa-miR-106a-5p, hsa-miR-106b-5p, hsa-miR-10a-5p, hsa-miR-10b-5p, hsa-miR-1180-3p, hsa-miR-1183, hsa-miR-122-5p, hsa-miR-1246, hsa-miR-1253, hsa-miR-125a-5p, hsa-miR-125b-5p, hsa-miR-100-5p, hsa-miR-548ah-5p, hsa-miR-98-5p, hsa-miR-4454, hsa-miR-128-3p, hsa-miR-1285-5p, hsa-miR-1268a, hsa-miR-1299, hsa-miR-1306-5p, hsa-miR-1290, hsa-miR-15a-5p, hsa-miR-132-3p, hsa-miR-323a-3p, hsa-miR-135a-5p, hsa-miR-135b-5p, hsa-miR-140-3p, hsa-miR-140-5p, hsa-miR-141-3p, hsa-miR-193a-5p, hsa-miR-143-3p, hsa-miR-144-3p, hsa-miR-126-3p, hsa-miR-199b-5p, hsa-miR-142-3p, hsa-miR-424-5p, hsa-miR-150-5p, hsa-miR-151a-3p, hsa-miR-152-3p, hsa-miR-1537-3p, hsa-miR-154-5p, hsa-miR-1972, hsa-miR-361-5p, hsa-miR-24-3p, hsa-miR-16-5p, hsa-miR-181a-2-3p, hsa-miR-181a-5p, hsa-miR-181c-5p, hsa-miR-1827, hsa-miR-186-5p, hsa-miR-187-3p, hsa-miR-4455, hsa-miR-190b, hsa-miR-191-5p, hsa-miR-1910-5p, hsa-miR-1915-3p, hsa-miR-193a-3p, hsa-miR-23b-3p, hsa-miR-193b-3p, hsa-miR-194-5p, hsa-miR-195-5p, hsa-miR-505-3p, hsa-miR-574-3p, hsa-miR-1976, hsa-miR-199a-3p, hsa-miR-199a-5p, hsa-miR-30e-3p, hsa-miR-19a-3p, hsa-miR-19b-3p, hsa-miR-200a-3p, hsa-miR-200b-3p, hsa-miR-203a-5p, hsa-miR-204-5p, hsa-miR-205-5p, hsa-miR-20a-5p, hsa-miR-21-5p, hsa-miR-548ar-5p, hsa-miR-214-3p, hsa-miR-146a-5p, hsa-miR-335-5p, hsa-miR-22-3p, hsa-miR-221-3p, hsa-miR-222-3p, hsa-miR-223-3p, hsa-miR-660-5p, hsa-miR-377-3p, hsa-miR-145-5p, hsa-miR-218-5p, hsa-miR-26a-5p, hsa-miR-26b-5p, hsa-miR-27a-3p, hsa-miR-27b-3p, hsa-miR-28-3p, hsa-miR-28-5p, hsa-miR-299-3p, hsa-miR-29a-3p, hsa-miR-495-3p, hsa-miR-29c-3p, hsa-miR-301a-3p, hsa-miR-302b-3p, hsa-miR-30a-3p, hsa-let-7g-5p, hsa-miR-30b-5p, hsa-miR-30c-5p, hsa-miR-127-3p, hsa-miR-4286, hsa-miR-30e-5p, hsa-miR-3158-3p, hsa-miR-3168, hsa-miR-3180-5p, hsa-miR-3195, hsa-miR-32-5p, hsa-miR-320a, hsa-miR-320e, hsa-let-7e-5p, hsa-miR-323b-3p, hsa-miR-324-5p, hsa-miR-148a-3p, hsa-miR-337-3p, hsa-miR-337-5p, hsa-miR-33a-5p, hsa-miR-340-5p, hsa-miR-342-3p, hsa-miR-34a-5p, hsa-miR-361-3p, hsa-miR-497-5p, hsa-miR-362-3p, hsa-miR-363-3p, hsa-miR-365a-3p, hsa-miR-664a-3p, hsa-miR-374a-5p, hsa-miR-376a-3p, hsa-miR-376c-3p, hsa-miR-378d, hsa-miR-378f, hsa-miR-378g, hsa-miR-378i, hsa-miR-379-5p, hsa-miR-382-5p, hsa-miR-423-3p, hsa-miR-423-5p, hsa-miR-130a-3p, hsa-miR-425-5p, hsa-miR-190a-5p, hsa-miR-2117, hsa-miR-548z, hsa-miR-25-3p, hsa-miR-1323, hsa-miR-4516, hsa-miR-451a, hsa-miR-487a-3p, hsa-miR-4532, hsa-miR-455-5p, hsa-miR-483-5p, hsa-miR-486-3p, hsa-miR-4443, hsa-miR-487b-3p, hsa-miR-491-5p, hsa-miR-543, hsa-miR-496, hsa-let-7f-5p, hsa-miR-499a-5p, hsa-miR-503-5p, hsa-miR-148b-3p, hsa-miR-509-3p, hsa-miR-509-5p, hsa-miR-513b-5p, hsa-miR-513c-5p, hsa-miR-514a-3p, hsa-miR-514a-5p, hsa-miR-514b-5p, hsa-miR-518b, hsa-miR-5196-3p, hsa-miR-526a, hsa-miR-548d-5p, hsa-miR-30a-5p, hsa-miR-30d-5p, hsa-miR-196a-5p, hsa-miR-136-5p, hsa-miR-4421, hsa-miR-548m, hsa-miR-548n, hsa-miR-99b-5p, hsa-miR-551b-3p, hsa-miR-556-3p, hsa-miR-23a-3p, hsa-miR-574-5p, hsa-miR-575, hsa-miR-578, hsa-miR-584-3p, hsa-miR-655-3p, hsa-miR-590-5p, hsa-miR-593-3p, hsa-miR-597-5p, hsa-miR-601, hsa-miR-607, hsa-miR-616-3p, hsa-miR-630, hsa-miR-663a, hsa-miR-7-5p, hsa-miR-764, hsa-miR-769-5p, hsa-miR-887-3p, hsa-miR-888-5p, hsa-miR-93-5p and/or hsa-miR-95-3p.
72. The method of any one of alternatives 67-71, wherein the miRNAs comprise any one or more of; hsa-let-7a-5p, hsa-miR-29b-3p, hsa-miR-99a-5p, hsa-miR-15b-5p, hsa-miR-548aa, hsa-miR-15a-5p, hsa-miR-193a-5p, hsa-miR-126-3p, hsa-miR-1972 and/or hsa-miR-196a-5p.
73. The method of any one of alternatives 67-72, wherein the miRNAs comprise any one or more of: hsa-let-7a-5p, hsa-miR-142-3p, hsa-miR-29b-3p and/or hsa-miR-99a-5p.
74. The method of any one of alternatives 67-73, wherein the biomarker or mediator of tissue damage comprise any one or more of: Complement C2, Complement C4b, Complement C5, Mannose Binding Lectin, Complement C1q, BDNF, Cathepsin D, NCAM, PAI-1 (Total), PDGF-AA, PDGF-AB/BB, RANTES, sICAM-1, sVCAM-1, FGF-2, Fractalkine, GROα, IFNα2, IL-1RA, IL-2, IL-3, IL-4, IL-8, IL-9, IL-10, IL-12p40, IL-13, IL-15, IP-10, MCP-1, MCP-3, MDC, MIP-1β, sCD40L, TNFα, TNFβ, 6CKine, APRIL, BCA-1, CCL28, CTACK, ENA-78, Eotaxin-2, Eotaxin-3, GCP-2, Granzyme A, Granzyme B, HMGB1, I-TAC, IFNβ, IFNω, IL-11, IL-16, IL-20, IL-24, IL-31, IL-33, IL-34, IL-35, LIF, MCP-2, MCP-4, MIP 1δ, MIP-3α, MIP-3β, MPIF-1, Perforin, sCD137, SDF-1, sFas, sFasL, TARC, TPO and/or TRAIL.
75. The method of any one of alternatives 67-74, wherein the biomarker or mediator of tissue damage comprises any one or more of: Complement C2, Complement C4b, Complement C1q, BDNF, PDGF-AB/BB, RANTES, sVCAM-1, Fractalkine, IFNα2, IL-9, MIP-1β, 6CKine, APRIL, BCA-1, CTACK, ENA-78, Eotaxin-3, GCP-2, IFNβ, IL-16, IL-20, IL-31, IL-33, IL-34, MCP-2, MIP-3α, MIP-3β, Perforin, SDF-1, TARC and/or TPO.
76. The method of any one of alternatives 67-75, wherein the biomarker or mediator of tissue damage comprises any one or more of: BDNF, C1QBP, C2, C4B, C5, CCL13, CCL15, CCL17, CCL19, CCL20, CCL22, CCL23, CCL26, CCL27, CCL3, CCL4, CCL5-2, CCL5, CCL8, CTSD, CX3CL1, CXCL10, CXCL11, CXCL12, CXCL13, CXCL5, FASLG, FGF2, GZMA, GZMB, HMGB1, ICAM1, IFNA2, IL16, IL1RN, IL20, IL33, IL34, IL9, LMAN1, NCAM, PDGFA, PDGFB, PRF1, SERPINE1, TNFRSF9, TNFSF13, TNFSF13B, TPO, TUBGCP2 and/or VCAM1.
77. The method of any one of alternatives 67-76, wherein the lectin is selected from the group consisting of Galanthus nivalis lectin (GNA), Narcissus pseudonarcissus lectin (NPA), Allium sativum lectin (ASA), Lens culinaris lectin (LCH), Sambucus nigra lectin (SNA), Maackia amurensis lectin (MAL), and concanavalin A.
78. The method of any one of alternatives 67-77, wherein the kidney is mammalian, such as human or porcine.
79. The method of alternative 78, wherein the kidney is human.
80. The method of any one of alternatives 67-79, wherein the lectin is immobilized on a support that comprises a material selected from the group consisting of Sepharose, agarose, latex, glass, thermoplastic polymers, polystyrene, polyethylene beads, polyvinyl, diatomaceous earth, nitrocellulose and silicon.
81. The method of any one of alternatives 67-80, wherein the vessel is a plate, such as a multi-well plate, a column, tube, flask, or beaker.
82. A method for connecting an organ to a circuit, configured to remove a pathogen, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans from said organ comprising:
attaching the organ, such as a kidney, liver, intestines, heart, lung, or pancreas to a vessel, such as an extracorporeal column, comprising a support, such as diatomaceous earth or an ion exchange resin, a lectin, such as Galanthus nivalis lectin (GNA), and/or an antibody preferably, wherein the lectin or antibody or both are immobilized on the support, such that when the organ is perfused with a perfusion solution, the perfusion solution exits the organ and is passed into the vessel comprising the support and when the perfusion solution exits the vessel comprising the support it is, optionally passed back through the organ, and preferably has a reduced amount of pathogen, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans than the perfusion solution had after exiting the organ prior to entering the vessel comprising the support.
83. The method of alternative 82, wherein the lectin is selected from the group consisting of Galanthus nivalis lectin (GNA), Narcissus pseudonarcissus lectin (NPA), Allium sativum lectin (ASA), Lens culinaris lectin (LCH), Sambucus nigra lectin (SNA), Maackia amurensis lectin (MAL), and concanavalin A.
84. The method of any one of alternatives 82 or 83, wherein the organ is a kidney and the kidney is mammalian, such as human or porcine.
85. The method of alternative 84, wherein the kidney is human.
86. The method of any one of alternatives 82-85, wherein the lectin is immobilized on a support that comprises a material selected from the group consisting of Sepharose, agarose, latex, glass, thermoplastic polymers, polystyrene, polyethylene beads, polyvinyl, diatomaceous earth, nitrocellulose and silicon.
87. The method of any one of alternatives 82-86, wherein the vessel is a plate, such as a multi-well plate, a column, tube, flask, or beaker.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments and are not intended to be limiting in scope.

FIG. 1 is a schematic illustration of a longitudinal cross section of an affinity cartridge.

FIG. 2 is a schematic illustration of a horizontal cross section at plane 2 in FIG. 1.

FIG. 3 is an illustration of a channel from FIG. 2. A hollow fiber membrane structure 40 is composed of a tubular section comprising a relatively tight ultrafiltration membrane 42 and relatively porous exterior portion 44 in which may be immobilized affinity molecules 46, such as lectins.

FIG. 4 is an illustrative representation of exosomal microRNAs associated with Ischemia-Reperfusion Injury, which are contemplated for removal by the devices described herein.

FIG. 5 is an illustrative representation of perfusate fluid from an explanted kidney passed over a packed column of GNA affinity resin.

FIG. 6 is an illustrative representation of perfusate fluid from an explanted kidney split into a shunted flow rate with a peristaltic pump at 200 ml/min passed over a packed column of GNA affinity resin.

FIG. 7 is an illustrative representation of the flow of fluid through 0.2 μm filter for the removal of particles larger than 200 nm from the perfusate fluid from an explanted kidney.

FIG. 8 is an illustrative representation of benchtop configuration of the exemplary device depicted in FIGS. 1-3 for circulating kidney perfusate over a packed column of GNA affinity resin under normal flow direction.

FIG. 9 is an illustrative representation of benchtop configuration of the exemplary device depicted in FIGS. 1-3 for circulating kidney perfusate over a packed column of GNA affinity resin under reverse flow direction.

FIG. 10 is an illustrative representation of a kidney perfusate passed over a column packed with a lectin affinity resin (e.g., GNA) followed by filtration through a 0.2 μm membrane.

FIG. 11 is an illustrative representation of a kidney perfusate passed over a packed column of GNA affinity resin under normal flow direction.

FIG. 12 is an illustrative representation of a kidney perfusate passed over a packed column of GNA affinity resin under side port flow direction.

FIG. 13 illustrates the measurement of 4 miRNAs (hsa-miR-let7a-5p, hsa-miR-142-3p, hsa-miR-29b-3p, and hsa-miR-99a-5p) in kidney perfusates following contact with a Hemopurifier®. FIGS. 13A, 13B, 13C and 13D illustrate the measurement of 4 miRNAs (hsa-miR-let7a-5p, hsa-miR-142-3p, hsa-miR-29b-3p, and hsa-miR-99a-5p) in kidney perfusates contacted with an exemplary device as shown in FIGS. 11-12.

FIG. 14 illustrates the qPCR measurement of a nucleic DNA marker (RNAseP) (FIG. 14A) and a mitochondrial DNA marker (7 s) (FIG. 14B) and the ratio of mitochondrial DNA/nuclear DNA copy number (FIG. 14C) in kidney perfusates following contact with a device comprising a column packed with a lectin affinity resin (e.g., GNA) followed by filtration through a 0.2 μm membrane as shown in FIG. 10.

FIG. 15 illustrates the Sytox green staining of the kidney perfusates passed over a column packed with a lectin affinity resin (e.g., GNA) followed by filtration through a 0.2 μm membrane as shown in FIG. 10 and a benchtop configuration of the exemplary device as shown in FIGS. 8-9.

FIG. 16 illustrates the quantitation of extracellular vesicles (EV) in the kidney perfusates passed over a column packed with a lectin affinity resin (e.g., GNA) followed by filtration through a 0.2 μm membrane as shown in FIG. 10 and a benchtop configuration of the exemplary device as shown in FIG. 8-9.

FIG. 17 illustrates the Sytox green staining and extracellular vesicles (EV) staining of the kidney perfusates passed over a column packed with a lectin affinity resin (e.g., GNA) followed by filtration through a 0.2 μm membrane as shown in FIG. 10 and a benchtop configuration of the exemplary device as shown in FIG. 8-9.

### DETAILED DESCRIPTION

Disclosed herein are methods for removing a pathogen, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof, from an organ, organ perfusate, a population of cells or a tissue or from the organ or tissue donor or recipient prior to, during and/or after transplantation. In some embodiments, the donor is a deceased donor. In some embodiments, the organ is donated by a donor, wherein the donation is after circulatory death (DCD). In some embodiments, the organ is donated by a donor, wherein the donation is after brain death (DBD).

### Definitions

Unless defined otherwise, all technical and scientific terms used herein have the same meaning as is commonly understood by one of ordinary skill in the art. All patents, applications, published applications and other publications referenced herein are expressly incorporated by reference in their entireties unless stated otherwise. In the event that there are a plurality of definitions for a term herein, those in this section prevail unless stated otherwise.

The articles “a” and “an” are used herein to refer to one or to more than one (for example, at least one) of the grammatical object of the article. By way of example, “an element” means one element or more than one element.

The terms “about” or “around” as used herein refer to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Unless defined otherwise, all technical and scientific terms used herein have the same meaning as is commonly understood by one of ordinary skill in the art to which this disclosure belongs. If there is a plurality of definitions for a term herein, those in this section prevail unless stated otherwise. The practice of the present disclosure will employ, unless indicated specifically to the contrary, conventional methods of molecular biology and recombinant DNA techniques within the skill of the art, many of which are described below for the purpose of illustration.

The terms “individual”, “subject”, or “patient” as used herein, means a human or a non-human mammal, e.g., a dog, a cat, a mouse, a rat, a cow, a sheep, a pig, a goat, a non-human primate, or a bird, e.g., a chicken, as well as any other vertebrate or invertebrate. The “individual”, “subject”, or “patient” may mean a human or a non-human mammal that is alive. The “individual”, “subject”, or “patient” may mean a human or a non-human mammal that is deceased.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

The terms “function” and “functional” as used herein refer to a biological, enzymatic, or therapeutic function.

The term “isolated” as used herein refers to material that is substantially or essentially free from components that normally accompany it in its native state. For example, an “isolated cell,” as used herein, includes a cell that has been purified from the milieu or organisms in its naturally occurring state, a cell that has been removed from a subject or from a culture, for example, it is not significantly associated with in vivo or in vitro substances.

The terms “treat”, “treating”, “treatment”, “therapeutic”, or “therapy” as used herein has its ordinary meaning as understood in light of the specification, and do not necessarily mean total cure or abolition of the disease or condition. The term “treating” or “treatment” as used herein (and as well understood in the art) also means an approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delaying or slowing of disease progression, amelioration or palliation of the disease state, diminishment of the reoccurrence of disease, and remission, whether partial or total and whether detectable or undetectable. “Treating” and “treatment” as used herein also include prophylactic treatment. Treatment methods comprise administering to a subject a therapeutically effective amount of an active agent or use of a therapeutic device. The administering step may consist of a single administration or may comprise a series of administrations. The compositions are administered or applied to the subject in an amount, for a duration, or for a number or repetitions sufficient to treat the patient. The length of the treatment period depends on a variety of factors, such as the severity of the condition, the age and genetic profile of the patient. It will also be appreciated that the treatment or prophylaxis may be modified over the course of a particular treatment or prophylaxis regime. In some instances, chronic administration or application may be required. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from or developing a disease or condition.

The term “inhibit” as used herein has its ordinary meaning as understood in light of the specification, and may refer to e.g., the reduction of a viral infection or sequela thereof or tissue rejection or the presence of extracellular vesicles or cytokines that mediate an inflammatory response. The reduction can be by 10%, 20%, 30%, 40%, 50%, 60%, 70%, 80%, 90%, or 100%, or an amount that is within a range defined by any two of the aforementioned values. As used herein, the term “delay” has its ordinary meaning as understood in light of the specification, and refers to a slowing, postponement, or deferment of an event, such as a viral infection, or a symptom or sequela thereof, or an inflammatory event to a time which is later than would otherwise be expected. The delay can be a delay of 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

The terms “nucleic acid” or “nucleic acid molecule” as used herein refers to polynucleotides, such as deoxyribonucleic acid (DNA) or ribonucleic acid (RNA), oligonucleotides, fragments generated by the polymerase chain reaction (PCR), or fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths.

The terms “peptide”, “polypeptide”, and “protein” as used herein refers to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths.

As used herein, a “lectin” is a protein that binds selectively to polysaccharides and glycoproteins. Although many are insufficiently specific to be useful, certain lectins are highly selective for enveloped viruses. Among lectins which have this property are those derived from Galanthus nivalis in the form of Galanthus nivalis agglutinin (“GNA”), Narcissus pseudonarcissus in the form of Narcissus pseudonarcissus agglutinin (“NPA”) and a lectin derived from blue green algae Nostoc ellipsosporum called “cyanovirin”. GNA is non-toxic and sufficiently safe that it has been incorporated into genetically engineered rice and potatoes (Bell et al. Transgenic Res 10(1): 35-42, 2001; Rao et al. Plant J 15(4): 469-477, 1998). These lectins bind to glycoproteins having a high mannose content such as found in HIV surface proteins (Chervenak et al. Biochemistry 34(16): 5685-5695, 1995).

As used herein, a “mannose glycoprotein” refers to a glycoprotein having mannose-mannose linkages in the form of α-1→3, α-1→6 mannose-mannose linkages, such as, high mannose, pauci-mannose, or oligomannose glycoproteins. High-mannose glycans typically contain between five and nine mannose residues attached to the chitobiose (GlcNAc2) core; with some of the mannose being alpha 1-3 or alpha 1-6. Pauci-mannose is an N-glycan moiety containing two core GlcNAc residues and two or three Mannose residues that do not bear oligosaccharide extensions. Oligomannose is a glycan that is an oligomer composed of mannose units.

As used herein, a “viral load” refers to the amount of viral particles, virus-like particles (VLP), or toxic fragments thereof in a biological fluid, such as blood, plasma or organ perfusion fluid. Viral load is accordingly related to the number of virus particles in the body Viral load can therefore be a measure of any of a variety of indicators of the presence of a virus, such as viral copy number per unit of blood, plasma or organ perfusion fluid or units of viral proteins or fragments thereof per unit of blood, plasma or organ perfusion fluid.

As used herein, the term “microRNA” or “miRNA” refers to highly conserved non-coding RNA molecules, about 21-25 nucleotides in length that play critical roles in regulating post-transcriptional gene expression by targeting messenger RNA (mRNA) of protein-coding genes. Mature miRNAs exist in the cellular cytoplasm as RNA duplexes to which an argonaute (Ago2) protein and a glycine-tryptophan repeat-containing protein bind, forming the core of a multi-subunit complex called the miRNA-mediating silencing complex (miRISC). The miRNA duplex contains two strands identified with either the suffix “-5p” (from the 5′ arm of pre-miRNA) or “-3p” (from the 3′ arm of the pre-miRNA). One of the strands of the duplex is typically discarded (passenger strand) while the retained strand guides mRNA target selection (guide strand). However, in some cases, two mature miRNAs excised from the 5′ and 3′ arms of the same stem-loop pre-miRNA have been reported to be functional and target on different mRNA. Once assembled into the miRISC through base-pairing interactions between nucleotides 2 and 8 of the miRNA and complementary nucleotides predominantly in the 3′-untranslated regions (UTR) of mRNAs, miRNAs act as repressors of translation. Each miRNA binds to many specific target mRNAs and often not to those in the same molecular pathway. miRNAs exist as both free-floating entities in plasma and other biofluids and also packaged into exosomes. It is believed that extracellular, non-miRNAs in circulation are by-products of dead cells stably bound to the Ago2 protein. Exosomes, which are produced in large quantities by diseased and activated cells, are packed with a full complement of the parent cell's cargo, including miRNA.

Some miRNAs have been identified as ubiquitously associated with various diseases. Specific miRNAs have been described as “fine-tuners” of immune responses that can either promote an inflammatory state or have anti-inflammatory effects by regulating toll-like receptor signaling. miRNAs are also non-antigenic, allowing them to escape immune surveillance, which can be beneficial for the virus. In viral infections, certain miRNAs may serve both as antiviral tools that stimulate the innate and adaptive immune systems while others have roles in viral propagation. Host miRNAs can be regulated by viruses and viruses can also usurp cellular resources to produce their own miRNAs that participate in immune evasion and maintaining infection. The presence of the various viral and host miRNAs may be monitored when evaluating the outcome of a therapeutic strategy.

As used herein, “ectosomes” are extracellular vesicles assembled at and released from the plasma membrane whereas exosomes are released on the exocytosis of multivesicular bodies (MVBs). Despite their considerable differences, the functions of ectosomes may be largely analogous to those of exosomes. In some contexts, the term ectosome is used interchangeably with extracellular vesicles or other terms known in the field, including, but not limited to microparticles, microvesicles, or shedding vesicles.

As used herein, “NETs” refer to neutrophil extracellular traps. NETs are released by activated neutrophils in the setting of signals including LPS, HMGB-1, pro-inflammatory cytokines, microorganisms and/or activated platelets, as well as, exosomes. NETs are released by neutrophils and are composed of DNA, histones, neutrophil granulation proteins, including myeloperoxidase and neutrophil elastase, and/or oxidant enzymes. NETs serve as microbial traps but also have been demonstrated to cause direct organ injury in a number of conditions.

As used herein “ceDNA” refers to circulating extracellular DNA and is produced by neutrophils in response to various stimuli including infections or inflammation. ceDNAs are present as NETs and are known to worsen the prognosis of various diseases. As used herein, “cytokines” refer to proteins or fragments or mutants thereof that modulate the immune system. Some non-limiting examples of cytokines include chemokines, interferons, interleukins, lymphokines and tumor necrosis factors. As used herein, “chemokines” or “chemotactic cytokines” are a family of small cytokines that are involved in cell migration.

### Biological Samples

Some embodiments provided herein include or utilize or analyze a biological sample, which can be referred to as a “sample.” Biological samples include, for example, cell culture media, organ perfusion fluid or perfusate with or without red blood cells or oxygen carriers, which may be near normothermic or hypothermic, including but not limited to Steen solution and University of Wisconsin solution and samples obtained from a subject. As used herein, the term “subject” includes an animal, a mammal, and a human or porcine. A sample, which may be obtained from a subject, can include any organ, tissue or fluid from the subject that may contain subcellular nanoparticles, a pathogen, viral particles, virus-like particles (VLPs), extracellular vesicles, such as exosomes or ectosomes, miRNAs, miRNAs, extracellular vesicle associated DNA, nucleic acids, such as DNA or RNA, circulating extracellular DNA (ceDNA), a biomarker or mediator of tissue damage, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof. Examples of such samples include cell culture media, organ perfusion fluid or perfusate with or without red blood cells or oxygen carriers, which may be near normothermic or hypothermic, including but not limited to Steen solution and University of Wisconsin solution, peripheral blood, sera, plasma, ascites, urine, cerebrospinal fluid (CSF), sputum, saliva, bone marrow, synovial fluid, aqueous humor, amniotic fluid, cerumen, breast milk, bronchioalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage or perfusion fluids. A biological sample may also include the blastocyl cavity, umbilical cord blood, or maternal circulation, which may be of fetal or maternal origin. In some embodiments, the sample may also include cell culture samples. Some non-limiting examples of cell culture samples include CAR T cell culture, stem cell culture, or pancreatic islet culture samples. The biological sample may also be a tissue sample or biopsy. In some embodiments, the sample comprises ascites fluid from a subject. In some embodiments, the sample comprises plasma from a subject. In some embodiments, the biological sample may be obtained from a subject that is alive. In some embodiments, the biological sample may be obtained from a subject that is deceased. In some embodiments, the biological sample may be obtained from an animal that will have organs harvested for purposes of xenotransplantation.

### Extracellular Vesicles, Exosomes and Ectosomes

Aspects of the invention concern the use of lectin affinity (e.g., employing a column having a substrate to which a lectin, such as Galanthus nivalis (GNA), is attached or a column packed with lectin affinity resin (e.g., GNA)) to remove extracellular vesicles (e.g., exosomes or ectosomes) from an organ donor or organ recipient prior to, during, or after transplantation or the organ itself after removal from the donor, such as during or after perfusion, which can be near normothermic or hypothermic perfusion. Some embodiments provided herein include a sample comprising extracellular vesicles, (e.g., exosomes or ectosomes). Generally, extracellular vesicles, (e.g., exosomes or ectosomes) are small vesicles that are released into the extracellular environment from a variety of different cells, for example, cells that originate from, or are derived from, the ectoderm, endoderm, or mesoderm including any such cells that have undergone genetic, environmental, and/or any other variations or alterations. An exosome, for example, is typically created intracellularly when a segment of the cell membrane spontaneously invaginates and is ultimately exocytosed (see e.g., Keller et al. (2006), Immunol. Lett. 107: 102-8, incorporated by reference herein in its entirety). In some embodiments, the extracellular vesicles (e.g., exosomes) have a diameter of greater than about 10 nm, 20 nm, or 30 nm; a diameter that is, or is about, 30-1000 nm, 30-800 nm, 30-200 nm, or 30-100 nm. In some embodiments, extracellular vesicles (e.g., exosomes) have a diameter of less than, or less than about, 10,000 nm, 1000 nm, 800 nm, 500 nm, 200 nm, 100 nm or 50 nm. In some contexts, the term extracellular vesicle is used interchangeably with exosomes or other terms known in the field, including, but not limited to, small extracellular vesicles, oncosomes, CytoVesicles, microvesicles, nanovesicles, vesicles, dexosomes, bleb, blebby, prostasomes, microparticles, intralumenal vesicles, endosomal-like vesicles or exocytosed vehicles. In some embodiments, said extracellular vesicles can comprise cytokines or chemokines as cargo and such extracellular vesicles can be removed using the technology set forth herein. Extracellular vesicles can also include any shed membrane bound particle that is derived from either the plasma membrane or an internal membrane. Extracellular vesicles can also include cell-derived structures bounded by a lipid bilayer membrane arising from both herniated evagination separation and sealing of portions of the plasma membrane or from the export of any intracellular membrane-bounded vesicular structure containing various membrane-associated proteins of tumor origin, including surface-bound molecules derived from the host circulation that bind selectively to the tumor-derived proteins together with molecules contained in the exosome lumen including tumor-derived microRNAs or intracellular proteins. Extracellular vesicles (e.g., exosomes or ectosomes) can also include membrane fragments.

In some embodiments, the extracellular vesicles (e.g., exosomes or ectosomes) are from subjects having transplant or tissue rejection or subjects believed to be at risk for ischemia-reperfusion injury (IRI), delayed graft function (DGF) and/or transplant or tissue rejection. In some embodiments, the transplant is a solid organ transplant. Some non-limiting examples of a solid organ transplant include kidney, liver, intestines, heart, lung, or pancreas. In some embodiments, the transplant is a transplantation of a population of cells. In some embodiments, the transplant is a kidney transplant. Some non-limiting examples of the population of cells transplant include a stem cell or a bone marrow transplant, pancreatic islet cells transplant, or chimeric antigen receptor T (CAR T) cells transplant. In some embodiments, the transplant rejection in a subject is hyperacute rejection. In some embodiments, the transplant rejection in a subject is acute rejection. In some embodiments, the transplant rejection in a subject is chronic rejection. In some embodiments, the transplant rejection is cell-mediated while in some embodiments the transplant rejection is antibody-mediated. In some embodiments, the extracellular vesicles (e.g., exosomes or ectosomes) are from samples having a delayed graft function (DGF) in kidney transplantation. In some embodiments, the extracellular vesicles (e.g., exosomes or ectosomes) are from samples having a transplant of normothermic machine perfused organs. In some embodiments, the extracellular vesicles (e.g., exosomes or ectosomes) are from samples having a transplant of hypothermic machine perfused organs. In some embodiments, the extracellular vesicles (e.g., exosomes or ectosomes) are from samples subjected to static cold storage. In some embodiments, the extracellular vesicles (e.g., exosomes or ectosomes) are from samples that have an Ischemia-reperfusion injury. In some embodiments, the extracellular vesicles (e.g., exosomes or ectosomes) are from samples having at least one or more of a delayed graft function (DGF), a transplant of normothermic machine perfused organs, transplanted with organs subjected to static cold storage and having an Ischemia-reperfusion injury.

In some embodiments, the extracellular vesicles (e.g., exosomes or ectosomes) are from an organ, a cell or a tissue donor. In some embodiments, the extracellular vesicles (e.g., exosomes or ectosomes) are from an organ, a population of cells or a tissue that has been removed from a donor. In some embodiments, the extracellular vesicles (e.g., exosomes or ectosomes) are from an organ, a population of cells or a tissue that has been transplanted into a recipient. In some embodiments, the extracellular vesicles (e.g., exosomes or ectosomes) are from an organ perfusate. In some embodiments, the extracellular vesicles (e.g., exosomes or ectosomes) are from a kidney perfusate. In some embodiments, the extracellular vesicles (e.g., exosomes or ectosomes) are from a recipient after an organ, a population of cells or a tissue has been transplanted.

miRNAs

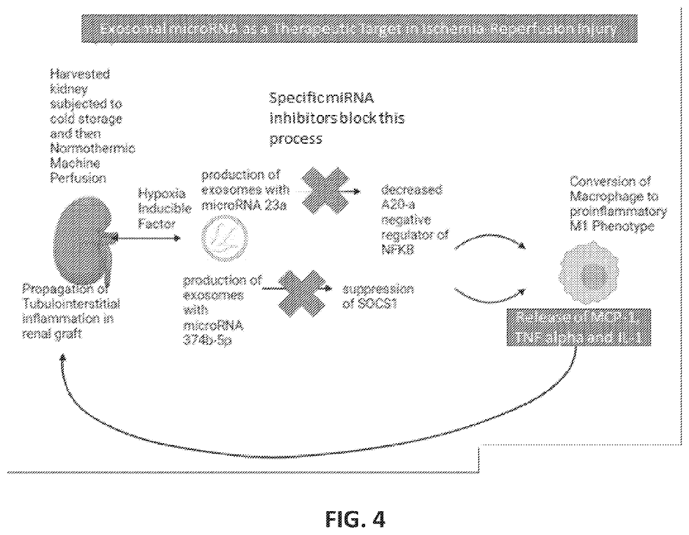

Aspects of the invention concern the use of lectin affinity (e.g., employing a column having a substrate, such as diatomaceous earth, to which a lectin such as GNA, NPA, SNA, and/or MAL is attached or a column comprising a lectin affinity resin (e.g., GNA)) to remove miRNAs from an organ, an organ perfusate, a population of cells or a tissue donor or an organ, a population of cells or a tissue recipient prior to, during, or after transplantation or the organ itself after removal from the donor such as during or after perfusion, which can be near normothermic or hypothermic perfusion. Some non-limiting examples of miRNAs include miR 23a, miR 374b-5p, miR28-5p and/or miR24-3p, miR-223, let-7e-5p, and/or miR-486-3p. Generally, delayed graft function (DGF) is defined as failure of the renal transplant to function immediately with a need for dialysis during the first week after transplantation. DGF affects approximately 30% of cases of renal transplantation. Ischemia-Reperfusion Injury is implicated in the development of DGF and exosomes containing miR 23a and/or miR 374b-5p are released in kidneys with ischemia-reperfusion injury as illustrated in FIG. 4. In some embodiments, the release of exosomes containing miR 23a and/or miR 374b-5p result in decreased regulation of NF-κB. In some embodiments, the release of exosomes containing miR 23a and/or miR 374b-5p result in suppression of Suppressor Of Cytokine Signaling 1 (SOCS1). In some embodiments, the release of exosomes containing miR 23a and/or miR 374b-5p results in conversion of macrophages to a pro-inflammatory M1 macrophages. In some embodiments, the conversion of macrophages results in release of pro-inflammatory cytokines, which cause tubulointerstitial inflammation in the kidneys. Accordingly, some embodiments concern methods of increasing the regulation of NF-κB, decreasing SOCS1 suppression, decreasing the conversion of macrophages to a pro-inflammatory M1 macrophages, or decreasing the release of pro-inflammatory cytokines in a subject having exosomes containing miR 23a and/or miR 374b-5p, wherein said subject receives an extracorporeal therapy, which utilizes any one or more of the lectin affinity devices described herein, such as a device comprising a column having a substrate, such as diatomaceous earth, to which a lectin such as GNA, NPA, SNA, and/or MAL is attached or a column packed with lectin affinity resin (e.g., GNA)). Such extracorporeal therapies and methods can include contacting blood and/or plasma or organ perfusion solution from said subject or harvested organ having exosomes containing miR 23a, miR 374b-5p, miR28-5p and/or miR24-3p with a column having a substrate, such as diatomaceous earth, to which a lectin such as GNA is attached or a column packed with lectin affinity resin (e.g., GNA)) for a time sufficient to bind said exosomes containing miR 23a, miR 374b-5p, miR28-5p and/or miR24-3p to said diatomaceous earth and/or lectin or a column packed with lectin affinity resin (e.g., GNA)) and reintroducing the blood and/or plasma or organ perfusion solution from said subject back into said subject or harvested organ, wherein the blood, plasma or organ perfusion fluid that is reintroduced, i.e., after contact with the column, has less exosomes containing miR 23a, miR 374b-5p, miR28-5p and/or miR24-3p than the blood or plasma or organ perfusion solution from said subject or organ prior to contact with the column. In some embodiments, the subject is identified or selected as one having exosomes containing miR 23a, miR 374b-5p, miR28-5p and/or miR24-3p prior to and/or after receiving the aforementioned extracorporeal therapy e.g., a sample of said subject's blood or plasma or organ perfusion solution is analyzed for the presence of exosomes containing miR 23a, miR 374b-5p, miR28-5p and/or miR24-3p before, during, and/or after receiving the aforementioned extracorporeal therapy. Such analysis can be performed by isolating exosomes from said subject or organ and analyzing the RNA present in said exosomes utilizing standard techniques in RNA analysis, such as sequencing or primer extension, or selective amplification after reverse transcription of the RNAs into cDNAs. In some embodiments the donor organ is placed on a perfusion circuit including the Hemopurifier®. Perfusate microRNAs are preferably measured before and during the procedure for a decline over time.

In some embodiments, exosomes containing miR 23a, miR 374b-5p, miR28-5p and/or miR24-3p are obtained utilizing one or more of the devices and methods set forth herein from subjects having transplant or tissue rejection or subjects believed to be at risk for ischemia-reperfusion injury (IRI), delayed graft function (DGF) and/or a transplant or tissue rejection. In some embodiments, the transplant is a solid organ transplant. Some non-limiting examples of the solid organ transplant include kidney, liver, intestines, heart, lung, or pancreas. In some embodiments, the transplant is a transplantation of a population of cells. In some embodiments, the transplant is a kidney transplant. Some non-limiting examples of the population of cells transplant include a stem cell or a bone marrow transplant, pancreatic islet cells transplant, or chimeric antigen receptor T (CAR T) cells transplant. In some embodiments, the transplant rejection in a subject is hyperacute rejection. In some embodiments, the transplant rejection in a subject is acute rejection. In some embodiments, the transplant rejection in a subject is chronic rejection. In some embodiments, the transplant rejection is cell-mediated while in some embodiments the transplant rejection is antibody-mediated. In some embodiments, exosomes containing miR 23a, miR 374b-5p, miR28-5p and/or miR24-3p are from samples having a delayed graft function (DGF) in kidney transplantation. In some embodiments, exosomes containing miR 23a, miR 374b-5p, miR28-5p and/or miR24-3p are obtained utilizing one or more of the devices and methods set forth herein from samples having a transplant of normothermic machine perfused organs. In some embodiments, exosomes containing miR 23a, miR 374b-5p, miR28-5p and/or miR24-3p are from samples having a transplant of hypothermic machine perfused organs. In some embodiments, exosomes containing miR 23a, miR 374b-5p, miR28-5p and/or miR24-3p are obtained utilizing one or more of the devices and methods set forth herein from samples subjected to static cold storage. In some embodiments, exosomes containing miR 23a and/or miR 374b-5p are from samples that have an Ischemia-reperfusion injury. In some embodiments, exosomes containing miR 23a, miR 374b-5p, miR28-5p and/or miR24-3p are obtained utilizing one or more of the devices and methods set forth herein from samples having at least one or more of a delayed graft function (DGF), a transplant of normothermic machine perfused organs, transplanted with organs subjected to static cold storage or having an Ischemia-reperfusion injury.

In some embodiments, exosomes containing miR 23a, miR 374b-5p, miR28-5p and/or miR24-3p are obtained utilizing one or more of the devices and methods set forth herein from an organ, an organ perfusate, a cell or a tissue donor. In some embodiments, exosomes containing miR 23a, miR 374b-5p, miR28-5p and/or miR24-3p are obtained utilizing one or more of the devices and methods set forth herein from an organ, an organ perfusate, a population of cells or a tissue that has been removed from a donor. In some embodiments, exosomes containing miR 23a, miR 374b-5p, miR28-5p and/or miR24-3p are obtained utilizing one or more of the devices and methods set forth herein from an organ, an organ perfusate, or a population of cells or a tissue that has been transplanted into a recipient. In some embodiments, exosomes containing miR 23a, miR 374b-5p, miR28-5p and/or miR24-3p are obtained utilizing one or more of the devices and methods set forth herein from a recipient after an organ, an organ perfusate, a population of cells or a tissue has been transplanted.

Aspects of the invention concern the use of lectin affinity (e.g., employing a column having a substrate, such as diatomaceous earth, to which a lectin such as GNA, NPA, SNA, and/or MAL is attached or a column comprising a lectin affinity resin (e.g., GNA) to remove miRNAs from an organ, an organ perfusate, a population of cells or a tissue donor or an organ donor, or a tissue recipient prior to, during, or after transplantation or the organ itself after removal from the donor such as during or after perfusion, which can be near normothermic or hypothermic perfusion. Some non-limiting examples of miRNAs that are removed include hsa-let-7a-5p, hsa-let-7b-5p, hsa-let-7c-5p, hsa-let-7d-5p, hsa-miR-29b-3p, hsa-miR-99a-5p, hsa-miR-15b-5p, hsa-miR-1-3p, hsa-miR-548aa, hsa-miR-106a-5p, hsa-miR-106b-5p, hsa-miR-10a-5p, hsa-miR-10b-5p, hsa-miR-1180-3p, hsa-miR-1183, hsa-miR-122-5p, hsa-miR-1246, hsa-miR-1253, hsa-miR-125a-5p, hsa-miR-125b-5p, hsa-miR-100-5p, hsa-miR-548ah-5p, hsa-miR-98-5p, hsa-miR-4454, hsa-miR-128-3p, hsa-miR-1285-5p, hsa-miR-1268a, hsa-miR-1299, hsa-miR-1306-5p, hsa-miR-1290, hsa-miR-15a-5p, hsa-miR-132-3p, hsa-miR-323a-3p, hsa-miR-135a-5p, hsa-miR-135b-5p, hsa-miR-140-3p, hsa-miR-140-5p, hsa-miR-141-3p, hsa-miR-193a-5p, hsa-miR-143-3p, hsa-miR-144-3p, hsa-miR-126-3p, hsa-miR-199b-5p, hsa-miR-142-3p, hsa-miR-424-5p, hsa-miR-150-5p, hsa-miR-151a-3p, hsa-miR-152-3p, hsa-miR-1537-3p, hsa-miR-154-5p, hsa-miR-1972, hsa-miR-361-5p, hsa-miR-24-3p, hsa-miR-16-5p, hsa-miR-181a-2-3p, hsa-miR-181a-5p, hsa-miR-181c-5p, hsa-miR-1827, hsa-miR-186-5p, hsa-miR-187-3p, hsa-miR-4455, hsa-miR-190b, hsa-miR-191-5p, hsa-miR-1910-5p, hsa-miR-1915-3p, hsa-miR-193a-3p, hsa-miR-23b-3p, hsa-miR-193b-3p, hsa-miR-194-5p, hsa-miR-195-5p, hsa-miR-505-3p, hsa-miR-574-3p, hsa-miR-1976, hsa-miR-199a-3p, hsa-miR-199a-5p, hsa-miR-30e-3p, hsa-miR-19a-3p, hsa-miR-19b-3p, hsa-miR-200a-3p, hsa-miR-200b-3p, hsa-miR-203a-5p, hsa-miR-204-5p, hsa-miR-205-5p, hsa-miR-20a-5p, hsa-miR-21-5p, hsa-miR-548ar-5p, hsa-miR-214-3p, hsa-miR-146a-5p, hsa-miR-335-5p, hsa-miR-22-3p, hsa-miR-221-3p, hsa-miR-222-3p, hsa-miR-223-3p, hsa-miR-660-5p, hsa-miR-377-3p, hsa-miR-145-5p, hsa-miR-218-5p, hsa-miR-26a-5p, hsa-miR-26b-5p, hsa-miR-27a-3p, hsa-miR-27b-3p, hsa-miR-28-3p, hsa-miR-28-5p, hsa-miR-299-3p, hsa-miR-29a-3p, hsa-miR-495-3p, hsa-miR-29c-3p, hsa-miR-301a-3p, hsa-miR-302b-3p, hsa-miR-30a-3p, hsa-let-7g-5p, hsa-miR-30b-5p, hsa-miR-30c-5p, hsa-miR-127-3p, hsa-miR-4286, hsa-miR-30e-5p, hsa-miR-3158-3p, hsa-miR-3168, hsa-miR-3180-5p, hsa-miR-3195, hsa-miR-32-5p, hsa-miR-320a, hsa-miR-320e, hsa-let-7e-5p, hsa-miR-323b-3p, hsa-miR-324-5p, hsa-miR-148a-3p, hsa-miR-337-3p, hsa-miR-337-5p, hsa-miR-33a-5p, hsa-miR-340-5p, hsa-miR-342-3p, hsa-miR-34a-5p, hsa-miR-361-3p, hsa-miR-497-5p, hsa-miR-362-3p, hsa-miR-363-3p, hsa-miR-365a-3p, hsa-miR-664a-3p, hsa-miR-374a-5p, hsa-miR-376a-3p, hsa-miR-376c-3p, hsa-miR-378d, hsa-miR-378f, hsa-miR-378g, hsa-miR-378i, hsa-miR-379-5p, hsa-miR-382-5p, hsa-miR-423-3p, hsa-miR-423-5p, hsa-miR-130a-3p, hsa-miR-425-5p, hsa-miR-190a-5p, hsa-miR-2117, hsa-miR-548z, hsa-miR-25-3p, hsa-miR-1323, hsa-miR-4516, hsa-miR-451a, hsa-miR-487a-3p, hsa-miR-4532, hsa-miR-455-5p, hsa-miR-483-5p, hsa-miR-486-3p, hsa-miR-4443, hsa-miR-487b-3p, hsa-miR-491-5p, hsa-miR-543, hsa-miR-496, hsa-let-7f-5p, hsa-miR-499a-5p, hsa-miR-503-5p, hsa-miR-148b-3p, hsa-miR-509-3p, hsa-miR-509-5p, hsa-miR-513b-5p, hsa-miR-513c-5p, hsa-miR-514a-3p, hsa-miR-514a-5p, hsa-miR-514b-5p, hsa-miR-518b, hsa-miR-5196-3p, hsa-miR-526a, hsa-miR-548d-5p, hsa-miR-30a-5p, hsa-miR-30d-5p, hsa-miR-196a-5p, hsa-miR-136-5p, hsa-miR-4421, hsa-miR-548m, hsa-miR-548n, hsa-miR-99b-5p, hsa-miR-551b-3p, hsa-miR-556-3p, hsa-miR-23a-3p, hsa-miR-574-5p, hsa-miR-575, hsa-miR-578, hsa-miR-584-3p, hsa-miR-655-3p, hsa-miR-590-5p, hsa-miR-593-3p, hsa-miR-597-5p, hsa-miR-601, hsa-miR-607, hsa-miR-616-3p, hsa-miR-630, hsa-miR-663a, hsa-miR-7-5p, hsa-miR-764, hsa-miR-769-5p, hsa-miR-887-3p, hsa-miR-888-5p, hsa-miR-93-5p and/or hsa-miR-95-3p. In some embodiments, the miRNAs that are removed include hsa-let-7a-5p, hsa-miR-142-3p, hsa-miR-29b-3p and/or hsa-miR-99a-5p. In some embodiments, hsa-let-7a-5p, hsa-miR-142-3p, hsa-miR-29b-3p, and/or hsa-miR-99a-5p play a role post renal transplantation in recipients. In some embodiments, hsa-let-7a-5p and/or hsa-miR-29b-3p are involved in development of fibrosis in ureteropelvic junction (UPJ) obstruction. In some embodiments, high levels of hsa-miR-142-3p are associated with impaired graft function (see e.g., Vahed et al. (2017), Int. Urol. Nephrol. 49: 1681-1689, incorporated by reference herein in its entirety). In some embodiments, hsa-miR-99a-5p is upregulated in subjects with acute rejection (see e.g., Tao et al. (2015), Transplantation Proceedings. 47 (6):1683-1687, incorporated by reference herein in its entirety). In some embodiments, hsa-miR-29b-3p is associated with chronic allograft dysfunction (see e.g., Chen et al. (2022), Int. J. Mol. Sci. 23(20): 12253, incorporated by reference herein in its entirety). In some embodiments, the miRNAs that are removed include hsa-let-7a-5p, hsa-miR-29b-3p, hsa-miR-99a-5p, hsa-miR-15b-5p, hsa-miR-548aa, hsa-miR-15a-5p, hsa-miR-193a-5p, hsa-miR-126-3p, hsa-miR-1972, and/or hsa-miR-196a-5p.

Accordingly, some embodiments concern methods of depleting miRNAs from an organ, an organ perfusate, a population of cells or a tissue donor or an organ donor, or a tissue recipient such as hsa-let-7a-5p, hsa-let-7b-5p, hsa-let-7c-5p, hsa-let-7d-5p, hsa-miR-29b-3p, hsa-miR-99a-5p, hsa-miR-15b-5p, hsa-miR-1-3p, hsa-miR-548aa, hsa-miR-106a-5p, hsa-miR-106b-5p, hsa-miR-10a-5p, hsa-miR-10b-5p, hsa-miR-1180-3p, hsa-miR-1183, hsa-miR-122-5p, hsa-miR-1246, hsa-miR-1253, hsa-miR-125a-5p, hsa-miR-125b-5p, hsa-miR-100-5p, hsa-miR-548ah-5p, hsa-miR-98-5p, hsa-miR-4454, hsa-miR-128-3p, hsa-miR-1285-5p, hsa-miR-1268a, hsa-miR-1299, hsa-miR-1306-5p, hsa-miR-1290, hsa-miR-15a-5p, hsa-miR-132-3p, hsa-miR-323a-3p, hsa-miR-135a-5p, hsa-miR-135b-5p, hsa-miR-140-3p, hsa-miR-140-5p, hsa-miR-141-3p, hsa-miR-193a-5p, hsa-miR-143-3p, hsa-miR-144-3p, hsa-miR-126-3p, hsa-miR-199b-5p, hsa-miR-142-3p, hsa-miR-424-5p, hsa-miR-150-5p, hsa-miR-151a-3p, hsa-miR-152-3p, hsa-miR-1537-3p, hsa-miR-154-5p, hsa-miR-1972, hsa-miR-361-5p, hsa-miR-24-3p, hsa-miR-16-5p, hsa-miR-181a-2-3p, hsa-miR-181a-5p, hsa-miR-181c-5p, hsa-miR-1827, hsa-miR-186-5p, hsa-miR-187-3p, hsa-miR-4455, hsa-miR-190b, hsa-miR-191-5p, hsa-miR-1910-5p, hsa-miR-1915-3p, hsa-miR-193a-3p, hsa-miR-23b-3p, hsa-miR-193b-3p, hsa-miR-194-5p, hsa-miR-195-5p, hsa-miR-505-3p, hsa-miR-574-3p, hsa-miR-1976, hsa-miR-199a-3p, hsa-miR-199a-5p, hsa-miR-30e-3p, hsa-miR-19a-3p, hsa-miR-19b-3p, hsa-miR-200a-3p, hsa-miR-200b-3p, hsa-miR-203a-5p, hsa-miR-204-5p, hsa-miR-205-5p, hsa-miR-20a-5p, hsa-miR-21-5p, hsa-miR-548ar-5p, hsa-miR-214-3p, hsa-miR-146a-5p, hsa-miR-335-5p, hsa-miR-22-3p, hsa-miR-221-3p, hsa-miR-222-3p, hsa-miR-223-3p, hsa-miR-660-5p, hsa-miR-377-3p, hsa-miR-145-5p, hsa-miR-218-5p, hsa-miR-26a-5p, hsa-miR-26b-5p, hsa-miR-27a-3p, hsa-miR-27b-3p, hsa-miR-28-3p, hsa-miR-28-5p, hsa-miR-299-3p, hsa-miR-29a-3p, hsa-miR-495-3p, hsa-miR-29c-3p, hsa-miR-301a-3p, hsa-miR-302b-3p, hsa-miR-30a-3p, hsa-let-7g-5p, hsa-miR-30b-5p, hsa-miR-30c-5p, hsa-miR-127-3p, hsa-miR-4286, hsa-miR-30e-5p, hsa-miR-3158-3p, hsa-miR-3168, hsa-miR-3180-5p, hsa-miR-3195, hsa-miR-32-5p, hsa-miR-320a, hsa-miR-320e, hsa-let-7e-5p, hsa-miR-323b-3p, hsa-miR-324-5p, hsa-miR-148a-3p, hsa-miR-337-3p, hsa-miR-337-5p, hsa-miR-33a-5p, hsa-miR-340-5p, hsa-miR-342-3p, hsa-miR-34a-5p, hsa-miR-361-3p, hsa-miR-497-5p, hsa-miR-362-3p, hsa-miR-363-3p, hsa-miR-365a-3p, hsa-miR-664a-3p, hsa-miR-374a-5p, hsa-miR-376a-3p, hsa-miR-376c-3p, hsa-miR-378d, hsa-miR-378f, hsa-miR-378g, hsa-miR-378i, hsa-miR-379-5p, hsa-miR-382-5p, hsa-miR-423-3p, hsa-miR-423-5p, hsa-miR-130a-3p, hsa-miR-425-5p, hsa-miR-190a-5p, hsa-miR-2117, hsa-miR-548z, hsa-miR-25-3p, hsa-miR-1323, hsa-miR-4516, hsa-miR-451a, hsa-miR-487a-3p, hsa-miR-4532, hsa-miR-455-5p, hsa-miR-483-5p, hsa-miR-486-3p, hsa-miR-4443, hsa-miR-487b-3p, hsa-miR-491-5p, hsa-miR-543, hsa-miR-496, hsa-let-7f-5p, hsa-miR-499a-5p, hsa-miR-503-5p, hsa-miR-148b-3p, hsa-miR-509-3p, hsa-miR-509-5p, hsa-miR-513b-5p, hsa-miR-513c-5p, hsa-miR-514a-3p, hsa-miR-514a-5p, hsa-miR-514b-5p, hsa-miR-518b, hsa-miR-5196-3p, hsa-miR-526a, hsa-miR-548d-5p, hsa-miR-30a-5p, hsa-miR-30d-5p, hsa-miR-196a-5p, hsa-miR-136-5p, hsa-miR-4421, hsa-miR-548m, hsa-miR-548n, hsa-miR-99b-5p, hsa-miR-551b-3p, hsa-miR-556-3p, hsa-miR-23a-3p, hsa-miR-574-5p, hsa-miR-575, hsa-miR-578, hsa-miR-584-3p, hsa-miR-655-3p, hsa-miR-590-5p, hsa-miR-593-3p, hsa-miR-597-5p, hsa-miR-601, hsa-miR-607, hsa-miR-616-3p, hsa-miR-630, hsa-miR-663a, hsa-miR-7-5p, hsa-miR-764, hsa-miR-769-5p, hsa-miR-887-3p, hsa-miR-888-5p, hsa-miR-93-5p and/or hsa-miR-95-3p, wherein said organ, organ perfusate, population of cells or tissue donor or organ donor, or a tissue recipient receives a therapy, which utilizes any one or more of the lectin affinity devices described herein, such as a device comprising a column having a substrate, such as diatomaceous earth, to which a lectin such as GNA, NPA, SNA, and/or MAL is attached or a column packed with lectin affinity resin (e.g., GNA). Such therapies and methods can include contacting blood and/or plasma organ or organ perfusion solution from said subject or harvested organ having miRNAs disclosed herein with a column having a substrate, such as diatomaceous earth, to which a lectin such as GNA is attached or a column packed with lectin affinity resin (e.g., GNA)) for a time sufficient to bind said miRNAs disclosed herein to said diatomaceous earth and/or lectin or a column packed with lectin affinity resin (e.g., GNA) and reintroducing the blood and/or plasma or organ perfusion solution from said subject back into said subject or harvested organ, wherein the blood, plasma or organ perfusion fluid that is reintroduced, i.e., after contact with the column, has less miRNAs disclosed herein than the blood or plasma or organ perfusion solution from said subject or organ prior to contact with the column. In some embodiments, the subject is identified or selected as one having miRNAs disclosed herein prior to and/or after receiving the aforementioned extracorporeal therapy e.g., a sample of said subject's blood or plasma or organ perfusion solution is analyzed for the presence miRNAs disclosed herein before, during, and/or after receiving the aforementioned extracorporeal therapy. Such analysis can be performed by isolating RNA from said subject or organ or organ perfusate utilizing standard techniques in RNA analysis, such as sequencing or primer extension, or selective amplification after reverse transcription of the RNAs into cDNAs. In some embodiments the donor organ is placed on a perfusion circuit including the Hemopurifier®. Perfusate microRNAs are preferably measured before and during the procedure for a decline over time. In some embodiments, the organ is a kidney.

In some embodiments, hsa-let-7a-5p, hsa-let-7b-5p, hsa-let-7c-5p, hsa-let-7d-5p, hsa-miR-29b-3p, hsa-miR-99a-5p, hsa-miR-15b-5p, hsa-miR-1-3p, hsa-miR-548aa, hsa-miR-106a-5p, hsa-miR-106b-5p, hsa-miR-10a-5p, hsa-miR-10b-5p, hsa-miR-1180-3p, hsa-miR-1183, hsa-miR-122-5p, hsa-miR-1246, hsa-miR-1253, hsa-miR-125a-5p, hsa-miR-125b-5p, hsa-miR-100-5p, hsa-miR-548ah-5p, hsa-miR-98-5p, hsa-miR-4454, hsa-miR-128-3p, hsa-miR-1285-5p, hsa-miR-1268a, hsa-miR-1299, hsa-miR-1306-5p, hsa-miR-1290, hsa-miR-15a-5p, hsa-miR-132-3p, hsa-miR-323a-3p, hsa-miR-135a-5p, hsa-miR-135b-5p, hsa-miR-140-3p, hsa-miR-140-5p, hsa-miR-141-3p, hsa-miR-193a-5p, hsa-miR-143-3p, hsa-miR-144-3p, hsa-miR-126-3p, hsa-miR-199b-5p, hsa-miR-142-3p, hsa-miR-424-5p, hsa-miR-150-5p, hsa-miR-151a-3p, hsa-miR-152-3p, hsa-miR-1537-3p, hsa-miR-154-5p, hsa-miR-1972, hsa-miR-361-5p, hsa-miR-24-3p, hsa-miR-16-5p, hsa-miR-181a-2-3p, hsa-miR-181a-5p, hsa-miR-181c-5p, hsa-miR-1827, hsa-miR-186-5p, hsa-miR-187-3p, hsa-miR-4455, hsa-miR-190b, hsa-miR-191-5p, hsa-miR-1910-5p, hsa-miR-1915-3p, hsa-miR-193a-3p, hsa-miR-23b-3p, hsa-miR-193b-3p, hsa-miR-194-5p, hsa-miR-195-5p, hsa-miR-505-3p, hsa-miR-574-3p, hsa-miR-1976, hsa-miR-199a-3p, hsa-miR-199a-5p, hsa-miR-30e-3p, hsa-miR-19a-3p, hsa-miR-19b-3p, hsa-miR-200a-3p, hsa-miR-200b-3p, hsa-miR-203a-5p, hsa-miR-204-5p, hsa-miR-205-5p, hsa-miR-20a-5p, hsa-miR-21-5p, hsa-miR-548ar-5p, hsa-miR-214-3p, hsa-miR-146a-5p, hsa-miR-335-5p, hsa-miR-22-3p, hsa-miR-221-3p, hsa-miR-222-3p, hsa-miR-223-3p, hsa-miR-660-5p, hsa-miR-377-3p, hsa-miR-145-5p, hsa-miR-218-5p, hsa-miR-26a-5p, hsa-miR-26b-5p, hsa-miR-27a-3p, hsa-miR-27b-3p, hsa-miR-28-3p, hsa-miR-28-5p, hsa-miR-299-3p, hsa-miR-29a-3p, hsa-miR-495-3p, hsa-miR-29c-3p, hsa-miR-301a-3p, hsa-miR-302b-3p, hsa-miR-30a-3p, hsa-let-7g-5p, hsa-miR-30b-5p, hsa-miR-30c-5p, hsa-miR-127-3p, hsa-miR-4286, hsa-miR-30e-5p, hsa-miR-3158-3p, hsa-miR-3168, hsa-miR-3180-5p, hsa-miR-3195, hsa-miR-32-5p, hsa-miR-320a, hsa-miR-320e, hsa-let-7e-5p, hsa-miR-323b-3p, hsa-miR-324-5p, hsa-miR-148a-3p, hsa-miR-337-3p, hsa-miR-337-5p, hsa-miR-33a-5p, hsa-miR-340-5p, hsa-miR-342-3p, hsa-miR-34a-5p, hsa-miR-361-3p, hsa-miR-497-5p, hsa-miR-362-3p, hsa-miR-363-3p, hsa-miR-365a-3p, hsa-miR-664a-3p, hsa-miR-374a-5p, hsa-miR-376a-3p, hsa-miR-376c-3p, hsa-miR-378d, hsa-miR-378f, hsa-miR-378g, hsa-miR-378i, hsa-miR-379-5p, hsa-miR-382-5p, hsa-miR-423-3p, hsa-miR-423-5p, hsa-miR-130a-3p, hsa-miR-425-5p, hsa-miR-190a-5p, hsa-miR-2117, hsa-miR-548z, hsa-miR-25-3p, hsa-miR-1323, hsa-miR-4516, hsa-miR-451a, hsa-miR-487a-3p, hsa-miR-4532, hsa-miR-455-5p, hsa-miR-483-5p, hsa-miR-486-3p, hsa-miR-4443, hsa-miR-487b-3p, hsa-miR-491-5p, hsa-miR-543, hsa-miR-496, hsa-let-7f-5p, hsa-miR-499a-5p, hsa-miR-503-5p, hsa-miR-148b-3p, hsa-miR-509-3p, hsa-miR-509-5p, hsa-miR-513b-5p, hsa-miR-513c-5p, hsa-miR-514a-3p, hsa-miR-514a-5p, hsa-miR-514b-5p, hsa-miR-518b, hsa-miR-5196-3p, hsa-miR-526a, hsa-miR-548d-5p, hsa-miR-30a-5p, hsa-miR-30d-5p, hsa-miR-196a-5p, hsa-miR-136-5p, hsa-miR-4421, hsa-miR-548m, hsa-miR-548n, hsa-miR-99b-5p, hsa-miR-551b-3p, hsa-miR-556-3p, hsa-miR-23a-3p, hsa-miR-574-5p, hsa-miR-575, hsa-miR-578, hsa-miR-584-3p, hsa-miR-655-3p, hsa-miR-590-5p, hsa-miR-593-3p, hsa-miR-597-5p, hsa-miR-601, hsa-miR-607, hsa-miR-616-3p, hsa-miR-630, hsa-miR-663a, hsa-miR-7-5p, hsa-miR-764, hsa-miR-769-5p, hsa-miR-887-3p, hsa-miR-888-5p, hsa-miR-93-5p and/or hsa-miR-95-3p are obtained utilizing one or more of the devices and methods set forth herein from subjects having transplant or tissue rejection or subjects believed to be at risk for ischemia-reperfusion injury (IRI), delayed graft function (DGF) and/or a transplant or tissue rejection or from the organ or organ perfusate. In some embodiments, the transplant is a solid organ transplant. Some non-limiting examples of the solid organ transplant include kidney, liver, intestines, heart, lung, or pancreas. In some embodiments, the transplant is a transplantation of a population of cells. In some embodiments, the transplant is a kidney transplant. Some non-limiting examples of the population of cells transplant include a stem cell or a bone marrow transplant, pancreatic islet cells transplant, or chimeric antigen receptor T (CAR T) cells transplant. In some embodiments, the transplant rejection in a subject is hyperacute rejection. In some embodiments, the transplant rejection in a subject is acute rejection. In some embodiments, the transplant rejection in a subject is chronic rejection. In some embodiments, the transplant rejection is cell-mediated while in some embodiments the transplant rejection is antibody-mediated. In some embodiments, miRNAs disclosed herein are from samples having a delayed graft function (DGF) in kidney transplantation. In some embodiments, miRNAs disclosed herein are obtained utilizing one or more of the devices and methods set forth herein from samples having a transplant of normothermic machine perfused organs. In some embodiments, miRNAs disclosed herein are from samples having a transplant of hypothermic machine perfused organs. In some embodiments, miRNAs disclosed herein are obtained utilizing one or more of the devices and methods set forth herein from samples subjected to static cold storage. In some embodiments, miRNAs disclosed herein are from samples that have an Ischemia-reperfusion injury. In some embodiments, miRNAs disclosed herein are obtained utilizing one or more of the devices and methods set forth herein from samples having at least one or more of a delayed graft function (DGF), a transplant of normothermic machine perfused organs, transplanted with organs subjected to static cold storage or having an Ischemia-reperfusion injury. In some embodiments, the organ is a kidney.

In some embodiments, miRNAs disclosed herein are obtained utilizing one or more of the devices and methods set forth herein from an organ, organ perfusate, a cell or a tissue donor. In some embodiments, miRNAs disclosed herein are obtained utilizing one or more of the devices and methods set forth herein from an organ, organ perfusate, a population of cells or a tissue that has been removed from a donor. In some embodiments, miRNAs disclosed herein are obtained utilizing one or more of the devices and methods set forth herein from an organ, or a population of cells or a tissue that has been transplanted into a recipient. In some embodiments, miRNAs disclosed herein are obtained utilizing one or more of the devices and methods set forth herein from a recipient after an organ, a population of cells or a tissue has been transplanted. In some embodiments, the organ is a kidney.

### Chemokines, Cytokines and Biomarkers or Mediators of Tissue Damage

Aspects of the invention concern the use of lectin affinity (e.g., employing a column having a substrate, such as diatomaceous earth, to which a lectin such as GNA, NPA, SNA, and/or MAL is attached or a column comprising a lectin affinity resin (e.g., GNA) to remove chemokines, cytokines and biomarkers or mediators of tissue damage from an organ, an organ perfusate, a population of cells or a tissue donor or an organ donor, a population of cells or a tissue recipient prior to, during, or after transplantation or the organ itself after removal from the donor such as during or after perfusion, which can be near normothermic or hypothermic perfusion. Some non-limiting examples of chemokines, cytokines and biomarkers or mediators of tissue damage that are removed include Complement C2, Complement C4b, Complement C5, Mannose Binding Lectin, Complement C1q, BDNF, Cathepsin D, NCAM, PAI-1 (Total), PDGF-AA, PDGF-AB/BB, RANTES, sICAM-1, sVCAM-1, FGF-2, Fractalkine, GROα, IFNα2, IL-1RA, IL-2, IL-3, IL-4, IL-8, IL-9, IL-10, IL-12p40, IL-13, IL-15, IP-10, MCP-1, MCP-3, MDC, MIP-1β, sCD40L, TNFα, TNFβ, 6CKine, APRIL, BCA-1, CCL28, CTACK, ENA-78, Eotaxin-2, Eotaxin-3, GCP-2, Granzyme A, Granzyme B, HMGB1, I-TAC, IFNβ, IFNω, IL-11, IL-16, IL-20, IL-24, IL-31, IL-33, IL-34, IL-35, LIF, MCP-2, MCP-4, MIP 1δ, MIP-3α, MIP-3β, MPIF-1, Perforin, sCD137, SDF-1, sFas, sFasL, TARC, TPO and/or TRAIL. In some embodiments, chemokines, cytokines and biomarkers or mediators of tissue damage that are removed include Complement C2, Complement C4b, Complement C1q, BDNF, PDGF-AB/BB, RANTES, sVCAM-1, Fractalkine, IFNα2, IL-9, MIP-1β, 6CKine, APRIL, BCA-1, CTACK, ENA-78, Eotaxin-3, GCP-2, IFNβ, IL-16, IL-20, IL-31, IL-33, IL-34, MCP-2, MIP-3α, MIP-3β, Perforin, SDF-1, TARC and/or TPO.

Accordingly, some embodiments concern methods of depleting chemokines, cytokines and biomarkers or mediators of tissue damage from an organ, an organ perfusate, a population of cells or a tissue donor or an organ donor, or a tissue recipient, such as Complement C2, Complement C4b, Complement C5, Mannose Binding Lectin, Complement C1q, BDNF, Cathepsin D, NCAM, PAI-1 (Total), PDGF-AA, PDGF-AB/BB, RANTES, sICAM-1, sVCAM-1, FGF-2, Fractalkine, GROα, IFNα2, IL-1RA, IL-2, IL-3, IL-4, IL-8, IL-9, IL-10, IL-12p40, IL-13, IL-15, IP-10, MCP-1, MCP-3, MDC, MIP-1β, sCD40L, TNFα, TNFβ, 6CKine, APRIL, BCA-1, CCL28, CTACK, ENA-78, Eotaxin-2, Eotaxin-3, GCP-2, Granzyme A, Granzyme B, HMGB1, I-TAC, IFNβ, IFNω, IL-11, IL-16, IL-20, IL-24, IL-31, IL-33, IL-34, IL-35, LIF, MCP-2, MCP-4, MIP 1δ, MIP-3α, MIP-3β, MPIF-1, Perforin, sCD137, SDF-1, sFas, sFasL, TARC, TPO and/or TRAIL, wherein said organ, organ perfusate, population of cells or tissue donor or organ donor, or tissue recipient receives an therapy, which utilizes any one or more of the lectin affinity devices described herein, such as a device comprising a column having a substrate, such as diatomaceous earth, to which a lectin such as GNA, NPA, SNA, and/or MAL is attached or a column packed with lectin affinity resin (e.g., GNA). Such extracorporeal therapies and methods can include contacting blood and/or plasma or organ perfusion solution from said subject or harvested organ having chemokines, cytokines and biomarkers or mediators of tissue damage disclosed herein with a column having a substrate, such as diatomaceous earth, to which a lectin such as GNA is attached or a column packed with lectin affinity resin (e.g., GNA) for a time sufficient to bind said chemokines, cytokines and biomarkers or mediators of tissue damage disclosed herein to said diatomaceous earth and/or lectin or a column packed with lectin affinity resin (e.g., GNA) and reintroducing the blood and/or plasma or organ perfusion solution from said subject back into said subject or harvested organ, wherein the blood, plasma or organ perfusion fluid that is reintroduced, i.e., after contact with the column, has less chemokines, cytokines and biomarkers or mediators of tissue damage disclosed herein than the blood or plasma or organ perfusion solution from said subject or organ prior to contact with the column. In some embodiments, the organ, organ perfusate, population of cells or tissue donor or organ donor, or tissue recipient is identified or selected as having chemokines, cytokines and biomarkers or mediators of tissue damage disclosed herein prior to and/or after receiving the aforementioned extracorporeal therapy e.g., a sample of said subject's blood or plasma or organ perfusion solution is analyzed for the presence of chemokines, cytokines and biomarkers or mediators of tissue damage disclosed herein before, during, and/or after receiving the aforementioned extracorporeal therapy. Such analysis can be performed by isolating proteins from said subject or organ or organ perfusate utilizing standard techniques and performing ELISA (Enzyme-linked immunosorbent assay), bead-based multiplex Luminex assays, aptamer-based assays, mass spectrometry or high throughput proteomics analysis. In some embodiments the donor organ is placed on a perfusion circuit allowing for contact with the lectin bound resin contained in for example an extracorporeal device. Perfusate chemokines, cytokines and biomarkers or mediators of tissue damage are preferably measured before and during the procedure for a decline over time. In some embodiments, the organ is a kidney.

In some embodiments, Complement C2, Complement C4b, Complement C5, Mannose Binding Lectin, Complement C1q, BDNF, Cathepsin D, NCAM, PAI-1 (Total), PDGF-AA, PDGF-AB/BB, RANTES, sICAM-1, sVCAM-1, FGF-2, Fractalkine, GROα, IFNα2, IL-1RA, IL-2, IL-3, IL-4, IL-8, IL-9, IL-10, IL-12p40, IL-13, IL-15, IP-10, MCP-1, MCP-3, MDC, MIP-1β, sCD40L, TNFα, TNFβ, 6CKine, APRIL, BCA-1, CCL28, CTACK, ENA-78, Eotaxin-2, Eotaxin-3, GCP-2, Granzyme A, Granzyme B, HMGB1, I-TAC, IFNβ, IFNω, IL-11, IL-16, IL-20, IL-24, IL-31, IL-33, IL-34, IL-35, LIF, MCP-2, MCP-4, MIP 1δ, MIP-3α, MIP-3β, MPIF-1, Perforin, sCD137, SDF-1, sFas, sFasL, TARC, TPO and/or TRAIL are obtained utilizing one or more of the devices and methods set forth herein from subjects having transplant or tissue rejection or subjects believed to be at risk for ischemia-reperfusion injury (IRI), delayed graft function (DGF) and/or a transplant or tissue rejection or from an organ, organ perfusate, population of cells or tissue donor or organ donor, or tissue recipient. In some embodiments, the transplant is a solid organ transplant. Some non-limiting examples of the solid organ transplant include kidney, liver, intestines, heart, lung, or pancreas. In some embodiments, the transplant is a transplantation of a population of cells. In some embodiments, the transplant is a kidney transplant. Some non-limiting examples of the population of cells transplant include a stem cell or a bone marrow transplant, pancreatic islet cells transplant, or chimeric antigen receptor T (CAR T) cells transplant. In some embodiments, the transplant rejection in a subject is hyperacute rejection. In some embodiments, the transplant rejection in a subject is acute rejection. In some embodiments, the transplant rejection in a subject is chronic rejection. In some embodiments, the transplant rejection is cell-mediated while in some embodiments the transplant rejection is antibody-mediated. In some embodiments, chemokines, cytokines and biomarkers or mediators of tissue damage disclosed herein are from samples having a delayed graft function (DGF) in kidney transplantation. In some embodiments, chemokines, cytokines and biomarkers or mediators of tissue damage disclosed herein are obtained utilizing one or more of the devices and methods set forth herein from samples having a transplant of normothermic machine perfused organs. In some embodiments, chemokines, cytokines and biomarkers or mediators of tissue damage disclosed herein are from samples having a transplant of hypothermic machine perfused organs. In some embodiments, chemokines, cytokines and biomarkers or mediators of tissue damage disclosed herein are obtained utilizing one or more of the devices and methods set forth herein from samples subjected to static cold storage. In some embodiments, chemokines, cytokines and biomarkers or mediators of tissue damage disclosed herein are from samples that have an Ischemia-reperfusion injury. In some embodiments, chemokines, cytokines and biomarkers or mediators of tissue damage disclosed herein are obtained utilizing one or more of the devices and methods set forth herein from samples having at least one or more of a delayed graft function (DGF), a transplant of normothermic machine perfused organs, transplanted with organs subjected to static cold storage or having an Ischemia-reperfusion injury. In some embodiments, the organ is a kidney.

In some embodiments, chemokines, cytokines and biomarkers or mediators of tissue damage disclosed herein are obtained utilizing one or more of the devices and methods set forth herein from an organ, organ perfusate, a cell or a tissue donor. In some embodiments, chemokines, cytokines and biomarkers or mediators of tissue damage disclosed herein are obtained utilizing one or more of the devices and methods set forth herein from an organ, organ perfusate, a population of cells or a tissue that has been removed from a donor. In some embodiments, chemokines, cytokines and biomarkers or mediators of tissue damage disclosed herein are obtained utilizing one or more of the devices and methods set forth herein from an organ, or a population of cells or a tissue that has been transplanted into a recipient. In some embodiments, chemokines, cytokines and biomarkers or mediators of tissue damage disclosed herein are obtained utilizing one or more of the devices and methods set forth herein from a recipient after an organ, a population of cells or a tissue has been transplanted. In some embodiments, the organ is a kidney.

Neutrophil Extracellular Traps (NETs) and their Components, DNA, Extracellular Vesicle Associated DNA, Histones, Neutrophil Granule Proteins and Oxidant Enzymes

Aspects of the invention also concern the use of lectin affinity (e.g., employing a column having a substrate, such as diatomaceous earth, to which a lectin such as GNA, NPA, SNA, and/or MAL is attached or a column comprising a lectin affinity resin (e.g., GNA)) to remove nucleic acids, such as DNA or RNA, circulating extracellular DNA (ceDNA), extracellular vesicle associated DNA, NETs and their components including DNA, histones, neutrophil granule proteins and/or oxidant enzymes, from an organ, a population of cells or a tissue donor or an organ, a population of cells or a tissue recipient prior to, during, or after transplantation or the organ itself after removal from the donor such as during or after perfusion, which can be near normothermic or hypothermic perfusion. NETS and circulating extracellular DNA (ceDNA) are released from activated neutrophils in response to stimuli including LPS, microorganisms, pro-inflammatory cytokines, activated platelets, exosomes and immune complexes. NETS are networks of extracellular fibers containing DNA, histones, the neutrophil granule proteins myeloperoxidase and neutrophil elastase, and/or oxidant enzymes. NETs serve as microbial traps but in excess are injurious to organs. Tissue injury is mediated, for example, through complement activation, exposed histone cytotoxicity, coagulation activation and/or propagation of neutrophil recruitment and activation. NETs have been implicated in acute lung injury and primary graft dysfunction. NETs have been detected in the perfusates of donor lungs undergoing ex vivo lung perfusion (EVLP) and was associated with worse recipient outcomes. NETs have further been implicated in ischemia-reperfusion injury and antibody medicated rejection in kidney transplants. In some embodiments, a donor organ is placed on a machine perfusion circuit containing the lectin affinity device (e.g., a Hemopurifier®) and NETs are measured over time in the perfusate to demonstrate a reduction over time. In other embodiments, the donor and/or the organ recipient will have their blood, plasma or organ perfusion fluid passed over the lectin affinity device (e.g., Hemopurifier®) to reduce the presence of NETs.

In some embodiments, the circulating extracellular DNA (ceDNA), extracellular vesicle associated DNA, NETs and/or their components including DNA, histones, neutrophil granule proteins and/or oxidant enzymes are isolated, using one or more of the lectin affinity devices set forth herein, from an organ, an organ perfusate, a cell or a tissue donor. In some embodiments, the circulating extracellular DNA (ceDNA), extracellular vesicle associated DNA, NETs and/or their components including DNA, histones, neutrophil granule proteins and/or oxidant enzymes are obtained utilizing one or more of the devices and methods set forth herein from an organ, an organ perfusate, a population of cells or a tissue that has been removed from a donor. In some embodiments, the circulating extracellular DNA (ceDNA), extracellular vesicle associated DNA, NETs and/or their components including DNA, histones, neutrophil granule proteins and/or oxidant enzymes are obtained utilizing one or more of the devices and methods set forth herein from an organ, an organ perfusate, a population of cells or a tissue that has been transplanted into a recipient. In some embodiments, the circulating extracellular DNA (ceDNA), extracellular vesicle associated DNA, NETs and/or their components including DNA, histones, neutrophil granule proteins and/or oxidant enzymes are obtained utilizing one or more of the devices and methods set forth herein from a recipient after an organ, an organ perfusate, a population of cells or a tissue has been transplanted.

Such extracorporeal therapies and methods can include contacting blood, plasma or organ perfusion solution from said subject or harvested organ having nucleic acids, such as DNA or RNA, circulating extracellular DNA (ceDNA), extracellular vesicle associated DNA, NETs and their components including DNA, histones, neutrophil granule proteins and/or oxidant enzymes with a column having a substrate, such as diatomaceous earth, to which a lectin such as GNA is attached, or to a column comprising a lectin affinity resin (e.g., GNA) for a time sufficient to bind said nucleic acids, such as DNA or RNA, circulating extracellular DNA (ceDNA), extracellular vesicle associated DNA, NETs and their components including DNA, histones, neutrophil granule proteins and/or oxidant enzymes to said diatomaceous earth and/or lectin or to a column comprising a lectin affinity resin (e.g., GNA) and reintroducing the blood, plasma or organ perfusion solution from said subject back into said subject or harvested organ, wherein the blood or plasma that is reintroduced, i.e., after contact with the column, has less nucleic acids, such as DNA or RNA, circulating extracellular DNA (ceDNA), extracellular vesicle associated DNA, NETs and their components including DNA, histones, neutrophil granule proteins and/or oxidant enzymes than the blood or plasma or organ perfusion solution from said subject or organ prior to contact with the column. In some embodiments, the subject is identified or selected as one having nucleic acids, such as DNA or RNA, circulating extracellular DNA (ceDNA), extracellular vesicle associated DNA, NETs and their components including DNA, histones, neutrophil granule proteins and/or oxidant enzymes prior to and/or after receiving the aforementioned extracorporeal therapy e.g., a sample of said subject's blood or plasma or organ perfusion solution is analyzed for the presence of nucleic acids, such as DNA or RNA, circulating extracellular DNA (ceDNA), extracellular vesicle associated DNA, NETs and their components including DNA, histones, neutrophil granule proteins and/or oxidant enzymes before, during, and/or after receiving the aforementioned extracorporeal therapy.

### Pathogens, Viral Particles, Virus-Like Particles (VLPs) and Fragments Thereof

Aspects of the invention concern the use of lectin affinity (e.g., employing a column having a substrate to which a lectin such as GNA is attached) to remove pathogens, viral particles, VLPs or fragments thereof from an organ, an organ perfusate, a population of cells or a tissue donor or an organ, an organ perfusate, a population of cells or a tissue recipient prior to, during, or after transplantation or the organ itself after removal from the donor such as during or after perfusion, which can be near normothermic or hypothermic perfusion. Some embodiments concern the use of lectin affinity (e.g., employing a column having a substrate to which a lectin such as GNA is attached or a column comprising a lectin affinity resin (e.g., GNA)) to remove pathogens, viral particles, VLPs or fragments thereof from an organ, an organ perfusate, cell population or a tissue to be transplanted. Some embodiments provided herein include a sample comprising pathogens, viral particles, virus-like particles (VLP), or fragments thereof. In some embodiments, the pathogens, viral particles, VLP or fragments thereof are from subjects having ischemia-reperfusion injury (IRI), delayed graft function (DGF) and/or a tissue or transplant rejection. In some embodiments, the transplant rejection is a solid organ transplant rejection. In some embodiments, the transplant rejection is a kidney transplant rejection. Some non-limiting examples of the solid organ transplant include kidney, liver, intestines, heart, lung, or pancreas. In other embodiments, the transplant rejection relates to a stem cell or a bone marrow transplant. In some embodiments, the transplant is a CAR T cell or pancreatic islet transplant. In some embodiments, the transplant rejection in the subject is a hyperacute rejection. In some embodiments, the transplant rejection in the subject is an acute rejection. In some embodiments, the transplant rejection in the subject is a chronic rejection.

In some embodiments, the pathogens, viral particles or fragments thereof are from an organ, an organ perfusate, a population of cells or a tissue donor. In some embodiments, the pathogens, viral particles are from an organ, an organ perfusate, a population of cells or a tissue that has been removed from a donor. In some embodiments, the pathogens, viral particles or fragments thereof are from an organ, an organ perfusate, a population of cells or a tissue that has been transplanted into a recipient. In some embodiments, the pathogens, viral particles or fragments thereof are from a recipient after an organ, an organ perfusate, a population of cells or a tissue has been transplanted.

Such extracorporeal therapies and methods can include contacting blood and/or plasma or organ perfusion solution from said subject or harvested organ having the pathogens, viral particles or fragments thereof with a column having a substrate, such as diatomaceous earth, to which a lectin such as GNA is attached, or to a column comprising a lectin affinity resin (e.g., GNA) for a time sufficient to bind said pathogens, viral particles or fragments thereof to said diatomaceous earth and/or lectin or to a column comprising a lectin affinity resin (e.g., GNA) and reintroducing the blood and/or plasma or organ perfusion solution from said subject back into said subject or harvested organ, wherein the blood, plasma or organ perfusion fluid that is reintroduced, i.e., after contact with the column, has less pathogens, viral particles or fragments thereof than the blood or plasma or organ perfusion solution from said subject or organ prior to contact with the column. In some embodiments, the subject is identified or selected as one having the pathogens, viral particles or fragments thereof prior to and/or after receiving the aforementioned extracorporeal therapy e.g., a sample of said subject's blood or plasma or organ perfusion solution is analyzed for the presence of the pathogens, viral particles or fragments thereof before, during, and/or after receiving the aforementioned extracorporeal therapy.

Removal of Pathogens, Viral Particles, VLP, and/or Extracellular Vesicles (e.g., Exosomes or Ectosomes), Inflammatory or Immunomodulatory Glycoproteins, Glycolipids or Lipoglycans, or Fragments Thereof

Some embodiments provided herein include removing pathogens, viral particles, virus-like particles (VLPs), extracellular vesicles, such as exosomes or ectosomes, inflammatory or immunomodulatory glycoproteins, glycolipids or lipoglycans, or fragments thereof from a biological sample obtained from an organ, an organ perfusate, cell population, or tissue from a donor or an organ, cell population, or tissue from a recipient, or from an organ, a population of cells or tissue itself prior to transplantation, such as during or after perfusion of the organ or tissue, which may be near normothermic or hypothermic. In some embodiments, pathogens, viral particles, virus-like particles (VLPs), extracellular vesicles, such as exosomes or ectosomes, inflammatory or immunomodulatory glycoproteins, lipoglycans, or glycolipids, or fragments thereof are isolated by contacting a sample with lectin immobilized on a substrate (e.g., a dish, microtiter plate, resin, fibers, membrane, or bead) for a time sufficient to allow the pathogens, viral particles, virus-like particles (VLPs), extracellular vesicles, such as exosomes or ectosomes, inflammatory or immunomodulatory glycoproteins, glycolipids or lipoglycans, or fragments thereof to bind to said lectin. In some embodiments, the lectin binds to a viral coat or envelope protein, or a fragment thereof. In some embodiments, the lectin binds to a bacterial protein, or a fragment thereof. In some embodiments, the lectin binds to Extracellular vesicles (e.g., Exosomes or Ectosomes), Inflammatory or Immunomodulatory Glycoproteins, Glycolipids or Lipoglycans, or fragments thereof. Examples of lectins useful with embodiments provided herein include, but not limited to, mannose-binding lectins. In some embodiments, the lectin is selected from the group consisting of Galanthus nivalis lectin (GNA), Narcissus pseudonarcissus lectin (NPA), Allium sativum lectin (ASA), Lens culinaris lectin (LCH), Sambucus nigra lectin (SNA), Maackia amurensis lectin (MAL), and concanavalin A. In some embodiments, the lectin is GNA, NPA, SNA, or MAL. In particular embodiments, the lectin is GNA.

In some embodiments, the lectins bind to the pathogens, viruses or VLPs in the organs, cells or tissues procured from porcine sources for xenotransplantation that include, but are not limited to, porcine cytomegalovirus (pCMV), porcine reseolovirus, porcine Retroviruses, porcine endogenous retroviruses (PERVs) A, B, C, porcine lymphotrophic herpesviruses (pLHV), or porcine circovirus.

In some embodiments, the lectins bind to the pathogens, viruses or VLPs to be removed in the organs, cells or tissues procured from human sources that include, but are not limited to Human Immunodeficiency Virus (HIV), Hepatitis C virus (HCV), Hepatitis B virus (HBV), Hepatitis D Virus (HDV); herpes family viruses such as, HTLV-1, HSV1/2, varicella zoster virus (VZV), Human herpesvirus (HHV)-6, HHV-7, HHV-8, EBV, Kaposi's sarcoma-associated herpesvirus (KSHV), and Cytomegalovirus (CMV), Human papilloma virus (HPV), Herpes Simplex Virus (HSV), picornaviruses including coronaviruses such as SARS-CoV2, SARS-CoV2 Spike Protein, SARS, MERS, OC43, 229E, NL63 and HKU1, porcine epidemic diarrhea virus (PEDV), Transmissible gastroenteritis virus (TGEV), enteroviruses such as EV-D68, A71, Coxsackie A/B, echoviruses, polioviruses, rhinoviruses or noroviruses.

In some embodiments, the lectins bind to inflammatory or immunomodulatory glycoproteins, or glycolipids or lipoglycans, such as LPS. In some embodiments, the lectins bind Mycoplasma or Ureaplasma. Some non-limiting examples of Mycoplasma include M. amphoriforme, M. buccale, M. faucium, M. fermentans, M. genitalium, M. hominis, M. incognitus, M. lipophilum, M. orale, M. penetrans, M. pirum, M. pneumoniae, M. primatum, M. salivarium, M. spermatophilum or Ureaplasma urealyticum.

In some embodiments, the exosomes and exosomal micro RNAs generated in Donors after circulatory death (DCD) and Donors after brain death (DBD) may be removed from the explanted organ or organ perfusate based on their binding affinity to lectin.

In some embodiments, the lectins or support (e.g., diatomaceous earth) on which the lectins are affixed or both or a column packed with lectin affinity resin (e.g., GNA)) in one or more of the affinity devices described herein bind to the extracellular vesicles (e.g., Exosomes or Ectosomes), Inflammatory or Immunomodulatory Glycoproteins, Glycolipids or Lipoglycans, or fragments thereof produced in transplants with ischemia-reperfusion injury. In some embodiments, the lectins or support (e.g., diatomaceous earth) on which the lectins are affixed or both in one or more of the affinity devices described herein bind to the extracellular vesicles (e.g., Exosomes or Ectosomes), Inflammatory or Immunomodulatory Glycoproteins, Glycolipids or Lipoglycans, or fragments thereof produced in transplants subjected to cold storage. In some embodiments, the lectins or support (e.g., diatomaceous earth) on which the lectins are affixed or both in one or more of the affinity devices described herein bind to the extracellular vesicles (e.g., Exosomes or Ectosomes), Inflammatory or Immunomodulatory Glycoproteins, Glycolipids or Lipoglycans, or fragments thereof produced in transplants subjected to static cold storage. In some embodiments, the lectins or support (e.g., diatomaceous earth) on which the lectins are affixed or both or a column packed with lectin affinity resin (e.g., GNA)) in one or more of the affinity devices described herein bind to the extracellular vesicles (e.g., Exosomes or Ectosomes), Inflammatory or Immunomodulatory Glycoproteins, Glycolipids or Lipoglycans, or fragments thereof produced in transplants subjected to normothermic machine perfusion. In some embodiments, the lectins or support (e.g., diatomaceous earth) on which the lectins are affixed or both or a column packed with lectin affinity resin (e.g., GNA)) in one or more of the affinity devices described herein bind to the extracellular vesicles (e.g., Exosomes or Ectosomes), Inflammatory or Immunomodulatory Glycoproteins, Glycolipids or Lipoglycans, or fragments thereof produced in transplants subjected to static cold storage and normothermic machine perfusion.

### Substrates

Some embodiments herein include a substrate also referred to as a support herein, which may be present or comprised in a vessel. In some embodiments, the substrate or support comprises a surface to which an extracellular vesicle binding material (e.g., exosome binding material, such as an antibody, lectin, ionic or non-ionic or ionic coating) or viral particle or viral particle fragment binding material (e.g., an antibody, lectin, ionic or non-ionic or ionic coating) is bound or immobilized. Substrates can include but are not limited to, for example, a surface, which may be in a vessel (e.g., a plate or microtiter well), beads, membranes, or fibers. A substrate can comprise appropriately functionalized matrices (e.g., ionic or non-ionic resins, Sepharose, latex, glass, polyethylene beads, polystyrene, polyvinyl, polyacrylamide, nitrocellulose, diatomaceous earth, zeolites or silicon).

### Therapy

In some embodiments described herein, methods of treatment or inhibition of organ or tissue transplant rejection are provided utilizing any one or more of the lectin affinity devices set forth herein. The methods can include removal of pathogens, viral particles, virus-like particles (VLPs), extracellular vesicles, such as exosomes or ectosomes, miRNAs, a biomarker or mediator of tissue damage, nucleic acids, such as DNA or RNA, extracellular vesicle associated DNA, circulating extracellular DNA (ceDNA), Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof, from an organ, an organ perfusate, cell population, or tissue prior to, during and/or after transplantation into a recipient utilizing any one or more of the lectin affinity devices set forth herein. Following removal of pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof, utilizing any one or more of the lectin affinity devices set forth herein, the transplant rejection or inflammatory response of the subject is reduced or inhibited. In some embodiments, utilizing any one or more of the lectin affinity devices set forth herein, the pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof are obtained from subjects that have experienced ischemia-reperfusion injury (IRI), delayed graft function (DGF) and/or organ, cell or tissue transplant rejection. In some embodiments, the transplant is a solid organ transplant. Some non-limiting examples of the solid organ transplant include kidney, liver, intestines, heart, lung, or pancreas. In some embodiments, the transplant is a transplantation of a population of cells. In some embodiments, the transplant is a kidney transplant. Some non-limiting examples of the population of cells transplant include a stem cell or a bone marrow transplant, pancreatic islet cells transplant, and chimeric antigen receptor T (CAR T) cells transplant. In some embodiments, the transplant rejection in a subject is a hyperacute rejection. In some embodiments, the transplant rejection in a subject is an acute rejection. In some embodiments, the transplant rejection in a subject is a chronic rejection. In some embodiments, the transplant rejection is cell-mediated while in some embodiments the transplant rejection is antibody-mediated. In some embodiments, utilizing any one or more of the lectin affinity devices set forth herein, the pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof are obtained from subjects having a delayed graft function (DGF) in kidney transplantation. In some embodiments, utilizing any one or more of the lectin affinity devices set forth herein, the pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof are obtained from samples having a transplant of normothermic machine perfused organs. In some embodiments, utilizing any one or more of the lectin affinity devices set forth herein, the pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof are obtained from samples subjected to static cold storage. In some embodiments, utilizing any one or more of the lectin affinity devices set forth herein, the pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof are obtained from samples that have an Ischemia-reperfusion injury. In some embodiments, utilizing any one or more of the lectin affinity devices set forth herein the pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof are obtained from samples having at least one or more of a delayed graft function (DGF), a transplant of normothermic machine perfused organs, subjected to static cold storage and having an Ischemia-reperfusion injury.

### Exemplary Lectin-Based Extracorporeal Devices

Disclosed herein are extracorporeal devices and methods of use for removing pathogens, viral particles, virus-like particles (VLPs), extracellular vesicles, such as exosomes or ectosomes, l miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof in a subject, wherein said subject is an organ, an organ perfusate, cell, or tissue donor. The extracorporeal devices comprise a lectin that binds to various glycoprotein-containing biological components, such as exosomes. When used for hemofiltration, the extracorporeal device with the lectin is able to filter, for example, pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof.

Once identified or selected as an organ, an organ perfusate, cell population, tissue, or subject (e.g., an organ, cell population, or tissue donor or recipient or isolated organ, cell population, or tissue) that would benefit from a lectin and/or antibody affinity therapy (e.g., Hemopurifier® therapy) utilizing the methods described herein, one can provide a lectin and/or antibody affinity therapy, such as Hemopurifier® therapy, to said identified or selected subjects. Such methods of selection or identification can be made by clinical evaluation or analysis of samples from said cell population, tissue, organ or subject (e.g., analysis of samples from said subject for the presence of pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof). Disclosed herein are extracorporeal devices and methods for the treatment or inhibition of organ, cell or tissue transplant rejection. Extracorporeal devices, such as the Hemopurifier®, comprise a lectin (e.g., GNA) and support (e.g., diatomaceous earth) that binds to various glycoprotein-containing biological components, such as pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof. In some embodiments, the extracorporeal devices comprise a packed column of lectin affinity resin (e.g., GNA) that binds to various glycoprotein-containing biological components, such as pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof. When used for hemofiltration, the extracorporeal device with the lectin and support (e.g., diatomaceous earth) or a packed column of lectin affinity resin (e.g., GNA) is able to filter and remove pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof and transplant rejection mediating nanoparticles.

For example, in some embodiments, the extracorporeal devices disclosed herein are useful in removing viral particles or fragments thereof that can lead to organ injury and death. In some embodiments, the extracorporeal devices disclosed herein are useful in removing extracellular vesicles (e.g., exosomes or ectosomes) from the organ, an organ perfusate, cell population, or tissue donor and/or the donor organ, cell population or tissue to be transplanted, resulting in reduced transplant rejections. This therapy may also or alternatively involve the depletion of extracellular vesicles (e.g., exosomes or ectosomes) from the transplant recipient after transplantation even if the recipient no longer rejects the transplanted cell(s), organ(s) or tissue(s).

In some embodiments, the extracorporeal devices disclosed herein comprising a lectin and support or a packed column of lectin affinity resin (e.g., GNA) are used for removing pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicles associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof in a subject may include the following steps: a) introducing a sample obtained from the subject prior to removal of an organ, an organ perfusate, population of cells, or tissue into a vessel, such as an extracorporeal device, comprising a lectin, such as GNA, NPA, SNA, and/or MAL, or an antibody or both immobilized therein on a support (e.g., diatomaceous earth) or into a packed column of lectin affinity resin (e.g., GNA); b) contacting the sample with the lectin or antibody or both and the support or a packed column of lectin affinity resin (e.g., GNA) in the vessel, such as an extracorporeal device; c) obtaining the sample obtained after b) for re-introduction into said subject or harvested organ, wherein the sample obtained after b) has a reduced amount of pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof, as compared to the sample of said subject prior to b); and d) optionally, selecting said subject for receiving an extracorporeal therapy accompanying organ, cell, or tissue transplantation, which removes pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof prior to (a) and/or e) optionally, screening for the presence of, e.g., detecting or identifying, pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof, in a sample from said subject, such as blood, plasma, prior to (a) or after (b) or both accompanying organ, cell, or tissue transplantation; and/or f) optionally, analyzing the glycan profile of extracellular vesicles, such as exosomes, ectosomes, or fragments thereof, from a sample from said subject prior to a) or after b) or both e.g., determining the ability of said extracellular vesicles, such as exosomes, ectosomes, or fragments thereof to bind to said lectin, such as GNA, NPA, SNA, and/or MAL, or said antibody and/or support (e.g., diatomaceous earth).

In some embodiments, the devices, systems and methods described herein comprise one or more hollow fiber cartridges containing an affinity agent that is a lectin, which preferably is GNA. Other lectins include NPA, Concanavalin A and cyanovirin. Examples of extracorporeal devices comprising lectins that can be used in the methods disclosed herein may be found in WO 2007/103572, WO 2009/023332, and WO 2010/065765, each of which is hereby expressly incorporated by reference in its entirety.

Accordingly, some embodiments relate to the use of extracorporeal devices comprising a lectin and a support such as diatomaceous earth (e.g., Hemopurifier® therapy), for removing viral particles or fragments thereof, extracellular vesicles (e.g., exosomes from blood or plasma or organ perfusion fluid from a subject or an organ to be transplanted), miRNAs, nucleic acids, such as DNA or RNA, circulating extracellular DNA (ceDNA), Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof. In some embodiments, the extracorporeal device comprises one or more hollow fiber cartridges comprising the lectin e.g., GNA. In accordance with hollow fiber membrane technology provided herein or otherwise known in the art, embodiments of the invention can involve a size exclusion mechanism for extracellular subcellular nanoparticles (including but not limited to viral particles, or fragments thereof, inflammatory mediating nanoparticles, extracellular vesicles, e.g., exosomes) to contact the affinity matrix, wherein larger blood components (including cells) are restricted from passing through the pores of the hollow fibers into the extra-capillary space of the device where the affinity agent resides. In some embodiments, the pore sizes range from 20-200 nanometers or 20-700 nanometers (e.g., 20 nm, 30 nm, 50 nm, 100 nm, 200 nm, 300 nm, 400 nm, 500 nm, 600 nm, or 700 nm). In some embodiments, the pore sizes are 200 nm or about 200 nm.

By way of example, blood, plasma, or the organ perfusion fluid is run through an extracorporeal circulation circuit that uses a hollow fiber cartridge with the membranes of said hollow fibers having sufficient permeability for the subcellular nanoparticles found in the blood, plasma or the organ perfusion fluid to be removed through the membrane of the hollow fibers and into an area outside of the fibers containing a substrate that is bound to a single or plurality of agents (e.g. lectins) capable of adhering to said viral particles or fragments thereof, subcellular nanoparticles or extracellular vesicles, (e.g., exosomes) in a manner such that said viral particles, subcellular nanoparticles extracellular vesicles (e.g., exosomes) are attached to said agent and do not substantially re-enter the hollow fibers. Within the knowledge of one skilled in the art are available numerous types of hollow fiber systems. Selection of said hollow fiber system is dependent on the desired blood, plasma or organ perfusion fluid volume and rate of passage of said blood, plasma or organ perfusion fluid volume through the hollow fiber system.

Regardless of the hollow fiber system used, the desirable approach is that said hollow fiber filters are required to allow passage of blood cells through the interior of said hollow fiber and allow diffusion of subcellular nanoparticles to the exterior. In order to allow such diffusion, the pores on the membrane of the hollow fiber need to be of a diameter sufficient to allow extracellular vesicles (e.g., exosomes ranging from the size of 20 nanometers to 700 nanometers in diameter), depending on the particles of interest. In some embodiments, the pores on the membrane of the hollow fiber need to be of a diameter sufficient to allow particles ranging from the size of 50 nanometers to 300 nanometers in diameter. In some embodiments, the pores on the membrane of the hollow fiber need to be of a diameter sufficient to allow particles ranging from the size of 80 nanometers to 700 nanometers in diameter.

The substrate, support, or matrix to be used with devices described herein preferably allows sufficient permeation of flow so that non-cellular blood components that enter the space exterior to the hollow fiber are distributed throughout the substrate or matrix material, so that substantial contact is made between the viral particles and/or extracellular vesicles (e.g., exosomes or ectosomes) permeating the hollow fiber filter and the binding agent that is attached to the substrate, support, or matrix. Suitable substrates, supports, or matrices are known to one skilled in the art. Said substrates, supports, or matrices include silica gel, dextran, agarose, nylon polymers, polymers of acrylic acid, co-polymers of ethylene and maleic acid anhydride, aminopropylsilica, aminocelite, polyethylene or glass beads, zeolite, diatomaceous earth, silicate containing diatomaceous earth or other substrates or matrices known in the art. Examples of such are described in the following patents, each of which are incorporated by reference herein in their entirety: Lentz U.S. Pat. No. 4,708,713, Motomura U.S. Pat. No. 5,667,684, Takashima et al U.S. Pat. No. 5,041,079, and Porath and Janson U.S. Pat. No. 3,925,152. The agents that are attached to said substrate may be chosen based on known affinity to viral particles and/or extracellular vesicles (e.g., exosomes or ectosomes).

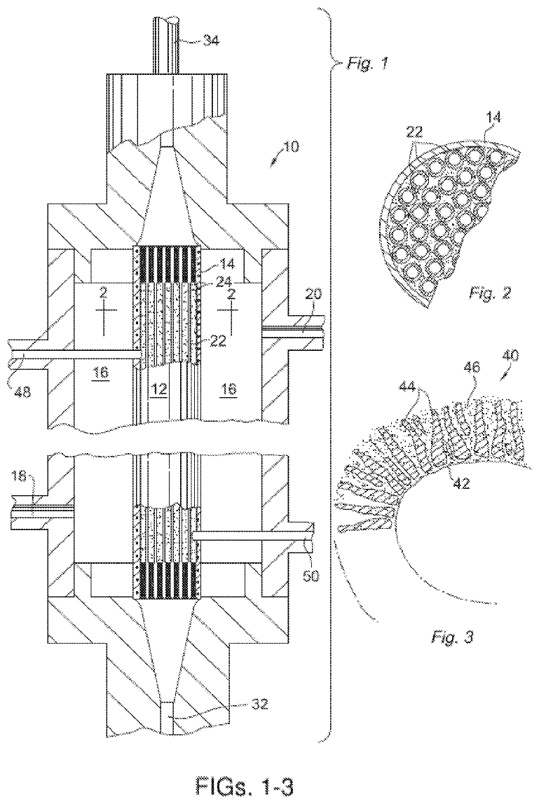

In some embodiments, the methods described herein are carried out by using an affinity cartridge using the device illustrated in FIG. 1. In this device, blood, plasma, or organ perfusate is passed through the lumen of a hollow fiber ultrafiltration membrane that is in intimate contact, on the non-blood wetted side of the membrane, with immobilized lectins, which accept and immobilize viral particles, virus-like particles (VLPs), extracellular vesicles, such as exosomes or ectosomes, miRNAs, nucleic acids, such as DNA or RNA, circulating extracellular DNA (ceDNA), Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof. Thus, the device retains intact glycoproteins (which may be a part of a larger structure) bound by lectin while allowing other components to pass through the lumen.

An exemplary device, described in detail in FIGS. 1-3, includes multiple channels of hollow fiber ultrafiltration membrane that forms a filtration chamber. An inlet port and an effluent port are in communication with the filtration chamber. The ultrafiltration membrane is preferably an anisotropic membrane with the tight or retention side facing the bloodstream. The membrane is conveniently formed of any number of polymers known to the art, for example, polysulfone, polyethersulfone, polyamides, polyimides, cellulose acetate, or polyacrylamide. Preferably, the membrane has pores 200-700 nm in diameter, which will allow passage of subcellular nanoparticles but not platelets or most blood cells (red blood cells, 2,000 nm diameter; lymphocytes, 7,000-12,000 nm diameter; macrophages, 10,000-18,000 nm diameter). A diagram of an exemplary device is shown in FIG. 1. The device comprises a cartridge 10 comprising a blood-processing chamber 12 formed of suitable material such as polycarbonate 14. Around chamber 12 is an optional exterior chamber 16. A temperature controlling fluid can be circulated into chamber 16 through port 18 and out of port 20. The device includes an inlet port 32 for the blood and an outlet port 34 for the effluent. The device also provides one or more ports 48 and 50, for accessing the extra-channel space in the cartridge. As shown in FIGS. 1 and 2, chamber 12 contains a plurality of ultrafiltration membranes 22. These membranes preferably have a 0.3 mm inside diameter and 0.5 mm outside diameter. FIG. 3 is a cross sectional representation of a channel 22 and shows the anisotropic nature of the membrane. As shown in FIG. 3, a hollow fiber membrane structure 40 is composed of a single polymeric material which is formed into a tubular section comprising a relatively tight ultrafiltration membrane 42 and relatively porous exterior portion 44 in which may be immobilized lectins 46. During the operation of the device, a solution containing the lectins is loaded on to the device through port 48. The lectins are allowed to immobilize to the exterior 22 of the membrane in FIG. 2. Unbound lectins can be collected from port 50 by washing with saline or other solutions. The cartridge housing is made of polycarbonate and the fibers are held in place with polyurethane potting material. The lectins are found in the extra-lumen (or extra-capillary or extra-channel) covalently bound to a solid material 10-200 microns (or wider) in diameter. The substrate material is made of a porous silicon dioxide material (SiO₂), preferably diatomaceous earth. Approximately 35-45 grams of lectin bound substrate is loaded into the extra-lumen space around the fibers through the polycarbonate side ports of the cartridge. During operation, blood runs along the length of the fibers and the plasma exits the pores and comes into contact with the lectin that is bound to the solid substrate.

For binding of lectins to the ultrafiltration membrane, the polymers of the ultrafiltration membrane are first activated (e.g., made susceptible for combining chemically with proteins) by using processes known in the art. Any number of different polymers and copolymers can be used. To obtain a reactive polyacrylic acid polymer, for example, carbodiimides can be used (Valuev et al., 1998, Biomaterials, 19:41-3). Once the polymer has been activated, the lectins can be attached directly or via a linker to form in either case an affinity matrix. Suitable linkers include, but are not limited to, avidin, strepavidin, biotin, protein A, or protein G. The lectins may also be directly bound to the polymer of the ultrafiltration membrane using coupling agents such as bifunctional reagents, or may be indirectly bound. In some embodiments, GNA covalently coupled to agarose can be used to form an affinity matrix.

Accordingly, in some of the methods described herein a lectin affinity device is used and said device comprises a filtration chamber configured to receive blood, plasma or organ perfusion fluid; a lectin, optionally coupled to agarose, diatomaceous earth, or aminocelite disposed within said filtration chamber; and a porous hollow fiber membrane, wherein said membrane has pores of 200-700 nm in diameter; wherein the lectin is selected from the group consisting of: Galanthus nivalis agglutinin (GNA), Narcissus pseudonarcissus agglutinin (NPA), cyanovirin, and Concanavalin A, and mixtures thereof wherein the cartridge is configured to remove viral particles, virus-like particles (VLPs), extracellular vesicles, such as exosomes or ectosomes, inflammatory or immunomodulatory glycoproteins, lipoglycans, or glycolipids, or fragments thereof from a sample (e.g., blood, plasma or organ perfusion fluid).

Processes that can be used to isolate lectins such as GNA are generally known in the art. For example, Van Damme et al. demonstrate isolating the lectin from Galanthus nivalis (snowdrop) bulbs by affinity purification with mannose or other sugars (Van Damme et al. FEBS Letters (1987) 215(1):140-144). Use of purified GNA for affinity purification purposes have been previously demonstrated, such as for isolating glycoproteins like immunoglobulins (Shibuya et al. Archives Biochem. Biophys. (1988) 267(2):676-680). Each of the references above are hereby expressly incorporated by reference in its entirety.

Aspects of the present invention also provide a device with a filtration chamber further comprises an inlet port and an outlet port; wherein a channel of said hollow fiber membrane is in fluidic communication with said inlet and said outlet ports; said cartridge having an extra-channel space within said chamber which surrounds said hollow fiber membrane; and wherein said lectin is, optionally, covalently coupled to agarose, diatomaceous earth or aminocelite that is disposed within said extra-channel space proximate to an exterior surface of said membrane.

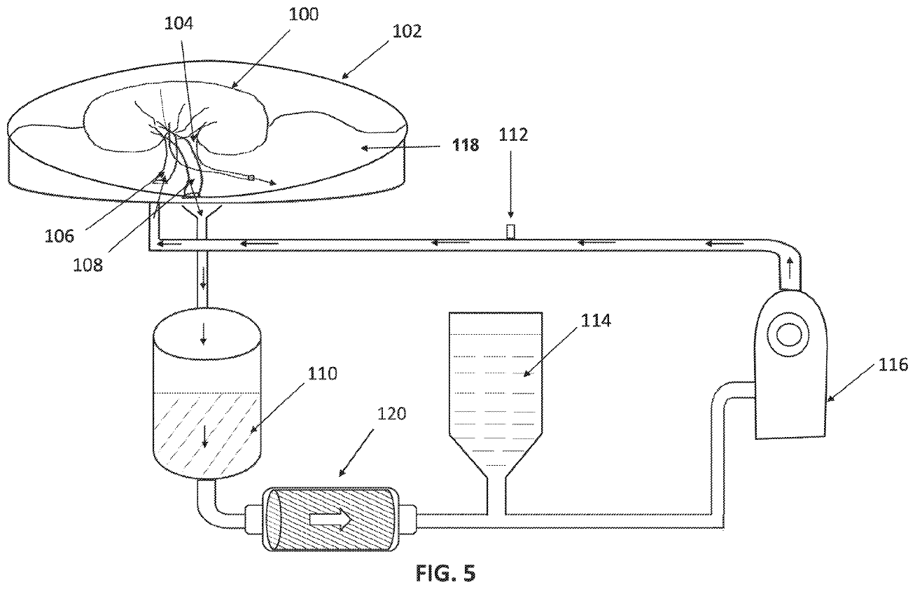

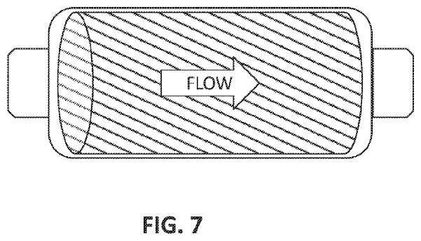

Aspects of the present invention also provide a device comprising a column packed with a lectin affinity resin (e.g., GNA). A diagram of an exemplary device comprising a packed column of lectin affinity resin is shown in FIG. 5. The device comprises an explanted kidney 100 placed in a basin 102. The basin comprises a chamber 118 that contains a reservoir from renal vein 108 and ureter 104. The explanted kidney 100 is connected directly to a packed column containing GNA affinity resin 110 followed by a 0.2 μm filter 120. An illustrative representation of the flow of fluid through the 0.2 μm filter 120 is shown in FIG. 7. A roller pump 116 supplies perfusion fluid 114 via the renal artery 106 to the kidney 100. The perfusion fluid will then exit the kidney 100 via the renal vein 108 and the ureter 104 and then enter the packed column containing GNA affinity resin 110 for removal of pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof, then pass the 0.2 μm filter 120 for the removal of particles larger than 200 nm such as, microvesicles, apoptotic bodies, oncosomes, and microorganisms before being returned to the renal artery 106 supplying the kidney 100. Perfusion fluid will be collected for pre-treatment and post-treatment assessment of pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof using a sample collection port 112.

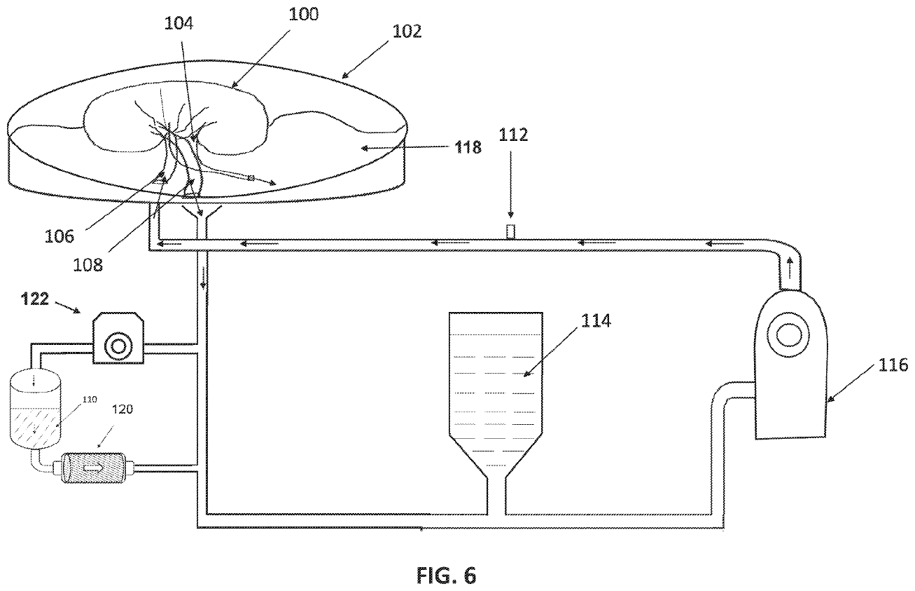

Aspects of the present invention also provide a device having the fluid path from the explanted device split into a shunted flow rate with a peristaltic pump at 200 ml/min with a column packed with a lectin affinity resin (e.g., GNA). A diagram of an exemplary device with a shunted flow and a packed column of lectin affinity resin is shown in FIG. 6. The device comprises an explanted kidney 100 placed in a basin 102. The basin comprises a chamber 118 that contains a reservoir from renal vein 108 and ureter 104. The explanted kidney 100 is connected directly to a packed column containing GNA affinity resin 110 followed by a 0.2 μm filter 120. A roller pump 116 supplies perfusion fluid 114 via the renal artery 106 to the kidney 100. The perfusion fluid will then exit the kidney 100 via the renal vein 108 and the ureter 104 and then fluid path will split into a shunted flow rate with a peristaltic pump at 200 ml/min 122 to a packed column containing GNA affinity resin 110 for removal of pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof, then pass the 0.2 μm filter 120 for the removal of particles larger than 200 nm such as, microvesicles, apoptotic bodies, oncosomes, and microorganisms before rejoining the fluid path to the renal artery 106 supplying the kidney 100. Perfusion fluid will be collected for pre-treatment and post-treatment assessment of pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof using a sample collection port 112.

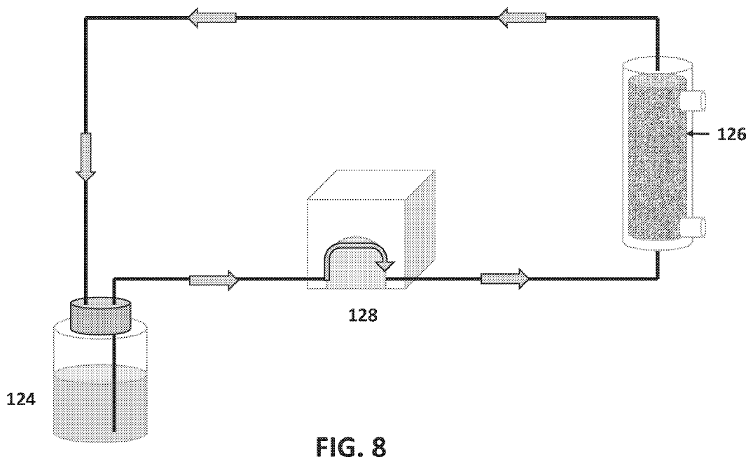

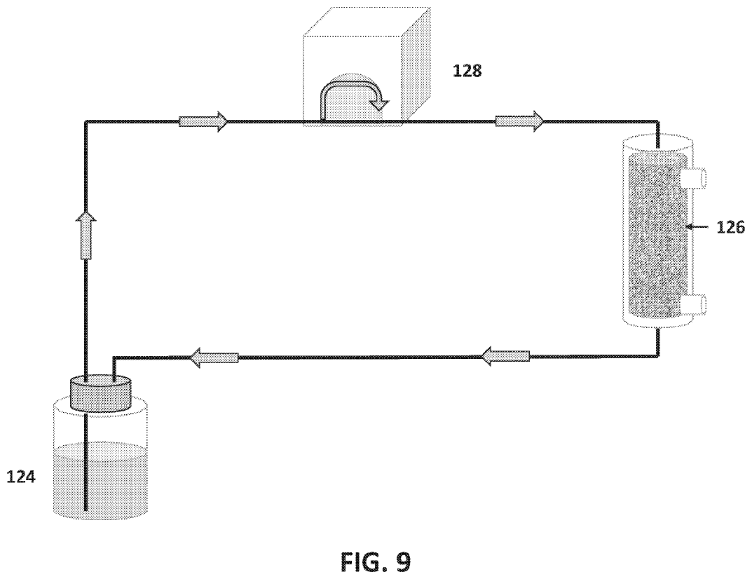

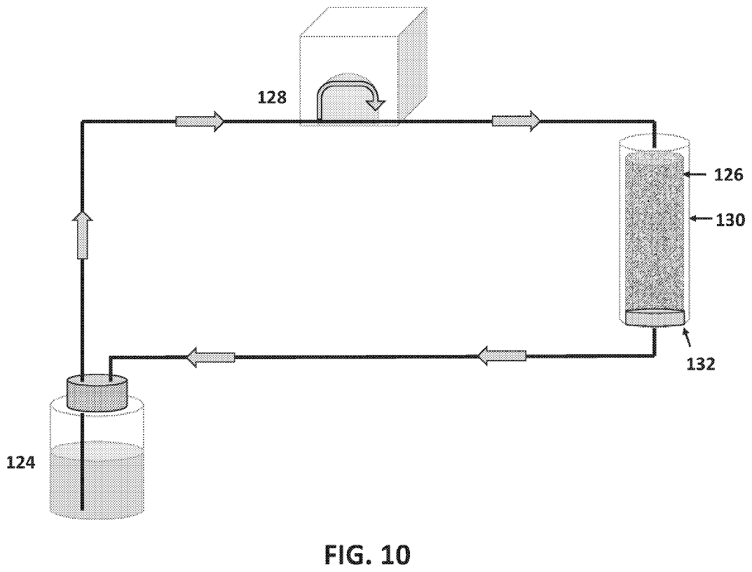

Aspects of the present invention also provide a benchtop configuration of the exemplary device depicted in FIGS. 1-3. A diagram of the benchtop configuration or down-scaled version or a mini-version of the exemplary device depicted in FIGS. 1-3 is shown in FIGS. 8 and 9. As shown in FIGS. 8 and 9, mini version of the device contains the same fiber material and filter pore size but representing about a 1/16th of the surface area of the exemplary device depicted in FIGS. 1-3. The device comprises a kidney perfusate 124 placed in a basin. The kidney perfusate 124 is circulated directly to a mini-Hemopurifier® cartridge circuit comprising a packed column containing GNA affinity resin 126. In some embodiments, 23.6 ml of kidney perfusate 124 is circulated directly to a mini-Hemopurifier® cartridge circuit comprising a packed column containing GNA affinity resin 126. A peristaltic pump at 19 ml/min 128 supplies perfusion fluid 124 to the mini-Hemopurifier® cartridge circuit 126 for removal of pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicles associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof. The perfusion fluid will then exit the mini-Hemopurifier® cartridge circuit 126 before being returned to the kidney perfusate 124 placed in a basin. In some embodiments, the kidney perfusate 124 is circulated directly to a mini-Hemopurifier® cartridge circuit comprising a packed column containing GNA affinity resin 126 for a total of 24 volume passes. In some embodiments, the kidney perfusate 124 is circulated directly to a mini-Hemopurifier® cartridge circuit comprising a packed column containing GNA affinity resin 126 for a total of about 2, about 4, about 6, about 8, about 10, about 12, about 14, about 16, about 18, about 20, about 22, about 24, about 26, about 28, about 30, about 40, about 50, about 70, about 100 or any number of volume passes between these values. FIG. 8 represents a normal flow of the perfusate into the mini-Hemopurifier® cartridge circuit comprising a packed column containing GNA affinity resin 126 while FIG. 9 represents a reverse flow of the perfusate into the mini-Hemopurifier® cartridge circuit comprising a packed column containing GNA affinity resin 126. Aspects of the present invention also provide a device comprising a column packed with a lectin affinity resin (e.g., GNA) followed by filtration through a 0.2 μm membrane. A diagram of an exemplary device comprising a packed column of lectin affinity resin is shown in FIG. 10. The device comprising a packed column of lectin affinity resin as shown in FIG. 10 does not place any size restriction on the components reaching the lectin affinity resin (e.g., GNA) as the components are filtered following their encounter with lectin affinity resin (e.g., GNA). The device comprises a kidney perfusate 124 placed in a basin. The kidney perfusate 124 is circulated directly to an affinity-resin packed column (e.g., GNA affinity resin) 126, which is a borosilicate 4 mL column 130 with a 20 μm polyethylene disc (DWK Life Sciences 4204001005) 132. In some embodiments, 23.6 ml of kidney perfusate 124 is circulated directly to an affinity-resin packed column (e.g., GNA affinity resin) 126. A peristaltic pump at 18 ml/min 128 supplies perfusion fluid 124 to the affinity-resin packed column (e.g., GNA affinity resin) 126 for removal of pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof. The perfusion fluid 124 will then exit the affinity-resin packed column (e.g., GNA affinity resin) 126 before being externally filtered using a 0.2 μm PES membrane filter to mimic the filtration effect of the exemplary devices of FIGS. 1-3 and 8-9 fibers. The perfusate 124 will then be returned to the kidney perfusate 124 placed in a basin. In some embodiments, the kidney perfusate 124 is circulated directly to an affinity-resin packed column (e.g., GNA affinity resin) 126 126 for a total of 24 volume passes. In some embodiments, the kidney perfusate 124 is circulated directly to an affinity-resin packed column (e.g., GNA affinity resin) 126 for a total of about 2, about 4, about 6, about 8, about 10, about 12, about 14, about 16, about 18, about 20, about 22, about 24, about 26, about 28, about 30, about 40, about 50, about 70, about 100 or any number of volume passes between these values.

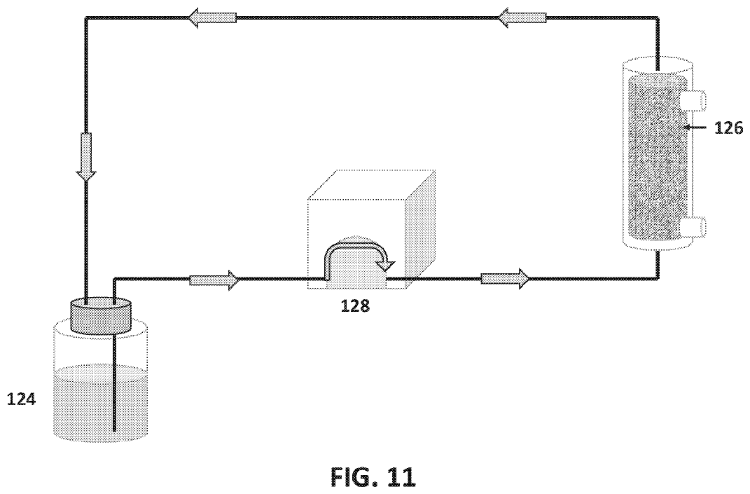

Aspects of the present invention also provide a device comprising a column packed with a lectin affinity resin (e.g., GNA). A diagram of an exemplary device comprising a packed column of lectin affinity resin is shown in FIG. 11. The device comprises a kidney perfusate 124 placed in a basin. The kidney perfusate 124 is circulated directly to a Hemopurifier® cartridge circuit comprising a packed column containing GNA affinity resin 126. In some embodiments, 250 ml of kidney perfusate 124 is circulated directly to an affinity-resin packed column (e.g., GNA affinity resin) 126. A peristaltic pump at 200 ml/min 128 supplies perfusion fluid 124 to the affinity-resin packed column (e.g., GNA affinity resin) 126 for removal of pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicles associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof. The perfusion fluid will then exit the Hemopurifier® cartridge circuit 126 before being returned to the kidney perfusate 124 placed in a basin. In some embodiments, the kidney perfusate 124 is circulated directly to a Hemopurifier® cartridge circuit comprising a packed column containing GNA affinity resin 126 for a total of 24 volume passes. In some embodiments, the kidney perfusate 124 is circulated directly to a Hemopurifier® cartridge circuit comprising a packed column containing GNA affinity resin 126 for a total of about 2, about 4, about 6, about 8, about 10, about 12, about 14, about 16, about 18, about 20, about 22, about 24, about 26, about 28, about 30, about 40, about 50, about 70, about 100 or any number of volume passes between these values. In some embodiments, the Hemopurifier® cartridge circuit comprises about 40 grams of GNA affinity resin. In some embodiments, the Hemopurifier® cartridge circuit comprises about 10 grams, about 20 grams, about 30 grams, about 40 grams, about 50 grams, about 60 grams, about 70 grams, about 80 grams, about 90 grams, about 100 grams of GNA affinity resin or any amount between these values. FIG. 11 represents a normal flow direction of the perfusate into the Hemopurifier® cartridge circuit 126 comprising a packed column containing GNA affinity resin 126.

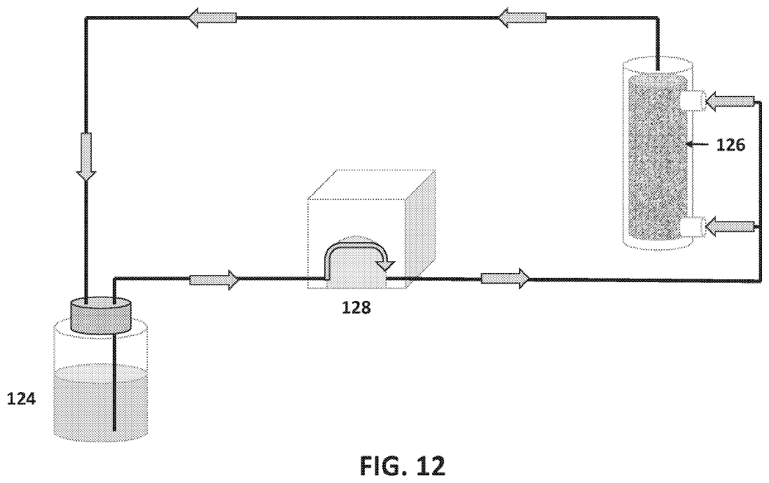

Aspects of the present invention also provide a device comprising a column packed with a lectin affinity resin (e.g., GNA). A diagram of an exemplary device comprising a packed column of lectin affinity resin with a side port flow direction is shown in FIG. 12. The device comprises a kidney perfusate 124 placed in a basin. The kidney perfusate 124 is circulated directly to a Hemopurifier® cartridge circuit comprising a packed column containing GNA affinity resin 126. In some embodiments, 250 ml of kidney perfusate 124 is circulated directly to an affinity-resin packed column (e.g., GNA affinity resin) 126. A peristaltic pump at 200 ml/min 128 supplies perfusion fluid 124 to the affinity-resin packed column (e.g., GNA affinity resin) 126 for removal of pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof. The perfusion fluid will then exit the Hemopurifier® cartridge circuit 126 before being returned to the kidney perfusate 124 placed in a basin. In some embodiments, the kidney perfusate 124 is circulated directly to a Hemopurifier® cartridge circuit comprising a packed column containing GNA affinity resin 126 for a total of 24 volume passes. In some embodiments, the kidney perfusate 124 is circulated directly to a Hemopurifier® cartridge circuit comprising a packed column containing GNA affinity resin 126 for a total of about 2, about 4, about 6, about 8, about 10, about 12, about 14, about 16, about 18, about 20, about 22, about 24, about 26, about 28, about 30, about 40, about 50, about 70, about 100 or any number of volume passes between these values. In some embodiments, the Hemopurifier® cartridge circuit comprises about 40 grams of GNA affinity resin. In some embodiments, the Hemopurifier® cartridge circuit comprises about 10 grams, about 20 grams, about 30 grams, about 40 grams, about 50 grams, about 60 grams, about 70 grams, about 80 grams, about 90 grams, about 100 grams of GNA affinity resin or any amount between these values. FIG. 12 represents a side port flow direction of the perfusate into the Hemopurifier® cartridge circuit 126 comprising a packed column containing GNA affinity resin 126.

For some methods described herein, blood, plasma, or organ perfusate from a subject or harvested organ having pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicles associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof is withdrawn from the subject and contacted with the device mentioned herein. In some embodiments, the blood is first separated into its plasma and cellular components. The blood, plasma or organ perfusion fluid is then contacted with the lectins to remove the viral particles and/or subcellular nanoparticles by binding between glycoproteins and lectins. The plasma can then be recombined with the cellular components and returned to the subject. Alternatively, the cellular components may be returned to the subject separately. In some embodiments, the time required to remove 50% of pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof is 5 minutes, 10 minutes, 15 minutes, 30 minutes, 45 minutes, 1, 2, 3, 4, 5, 6, 7, 8, 9, 10, 11, 12, 13, 14, 15, 16, 17, 18, 19, 20, 21, 22, 23, or 24 hours, or any amount of time within a range defined by any two of the aforementioned times. In some embodiments, the percentage of pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof are removed in 1, 2, 3, 4, 5, 6, 7, 8, 9, or 10 hours or any amount of time within a range defined by any two of the aforementioned times is 1, 2, 3, 4, 5, 10, 20, 30, 40, 50, 60, 70, 80, 90, or 100% or any percentage within a range defined by any two of the aforementioned values. The therapy can be repeated periodically until a desired response has been achieved. In some embodiments, the therapy can be carried out for 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, or 24 hours within a 24 hour period, or any amount of time within a range defined by any two of the aforementioned times. In some embodiments, the therapy can be repeated every day for 1, 2, 3, 4, 5, 6, 7, 8, 9, 10, 11, 12, 13, 14, or 15 days.

For some methods described herein, blood, plasma, or organ perfusion fluid, or organ perfusate (which can be near normothermic or hypothermic) having pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof is withdrawn from an organ and contacted with the device mentioned herein. In some embodiments, the blood, plasma, or organ perfusion fluid, or organ perfusate (which can be near normothermic or hypothermic) is first separated into its components. The blood, plasma, or organ perfusion fluid, or organ perfusate (which can be near normothermic or hypothermic) is then contacted with the lectin support or the column packed with a lectin affinity resin(e.g., GNA) to remove the pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof by binding to the lectin on the support. The blood, plasma, or organ perfusion fluid, or organ perfusate (which can be near normothermic or hypothermic) can then be recombined with the earlier isolated cellular components and returned to the organ. Alternatively, the cellular components may be returned to the organ separately. The therapy can be repeated periodically until a desired response has been achieved. In some embodiments, the therapy can be carried out for 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, or 24 hours within a 24 hour period, or any amount of time within a range defined by any two of the aforementioned times. In some embodiments, the therapy can be repeated every day for 1, 2, 3, 4, 5, 6, 7, 8, 9, 10, 11, 12, 13, 14, or 15 days.

In some embodiments, the methods and devices described herein additionally comprise affinity agents that are antibodies (e.g., monoclonal antibodies) that bind to pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof. In certain embodiments, the methods and devices utilized comprise a GNA or other lectin affinity agent and a monoclonal antibody affinity agent.

In some embodiments, the removal of pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof using a lectin affinity device as described herein (e.g., Hemopurifier® therapy) may include the following steps: a) providing a hollow fiber cartridge comprising a lectin or other affinity binding agent on a support (e.g., diatomaceous earth) or a column packed with a lectin affinity resin (e.g., GNA) that selectively binds to the pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof; b) removing a biological sample (e.g., blood or plasma or perfusate) from a subject or an organ using the system, the biological sample having a concentration of the pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof; c) processing the biological sample using the hollow fiber cartridge or a column packed with a lectin affinity resin (e.g., GNA) such that the affinity agents are in contact with the biological sample; d) capturing at least a portion of pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof from the biological sample such that said portion of the pathogens, viral particles, virus-like particles (VLPs), extracellular vesicles, such as exosomes or ectosomes, inflammatory or immunomodulatory glycoproteins, lipoglycans, or glycolipids, or fragments thereof is retained in the hollow fiber cartridge; and e) reintroducing the biological sample without said portion of captured pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof to the subject or an organ without removing the biological sample from the system before the biological sample is ready to be administered to the subject or the organ. Optionally, a biological sample from said subject or an organ, such as blood or plasma or perfusate, from said subject or organ is obtained before or after the therapy or both and said biological sample is analyzed for the level or amount of the pathogens, viral particles, virus-like particles (VLPs), extracellular vesicles, such as exosomes or ectosomes, inflammatory or immunomodulatory glycoproteins, lipoglycans, or glycolipids, or fragments thereof.

In some embodiments, a lectin and support (e.g., diatomaceous earth), comprised in an extracorporeal device or a column packed with a lectin affinity resin (e.g., GNA), is used to remove pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof in accordance with the following steps: a) introducing a sample obtained from the subject prior to removal of an organ, an organ perfusate, population of cells, or tissue into a vessel, such as an extracorporeal device, comprising a lectin, such as GNA, NPA, SNA, and/or MAL, or an antibody or both immobilized therein on a support (e.g., diatomaceous earth) or a column packed with a lectin affinity resin (e.g., GNA); b) contacting the sample with the lectin or antibody or both on the support or a column packed with a lectin affinity resin (e.g., GNA) in the vessel, such as in an extracorporeal device (e.g., a Hemopurifier®); c) obtaining the sample obtained after b) for re-introduction into said subject, wherein the sample obtained after b) has a reduced amount of pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof, as compared to the sample of said subject prior to b); and d) optionally, selecting said subject for receiving an extracorporeal therapy accompanying organ, an organ perfusate, cell, or tissue transplantation, which removes pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof prior to (a) and/or e) optionally, screening for the presence of e.g., detecting or identifying, pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof, in a sample from said subject, such as blood or plasma or organ perfusion fluid, prior to (a) or after (b) or both accompanying organ, cell, or tissue transplantation; and/or f) optionally, analyzing the glycan profile of extracellular vesicles, such as exosomes, ectosomes, or fragments thereof, from a sample from said subject prior to a) or after b) or both e.g., determining the ability of said extracellular vesicles, such as exosomes, ectosomes, or fragments thereof to bind to said lectin, such as GNA, NPA, SNA, and/or MAL, or said antibody or said support or all three.

One skilled in the art will recognize that a biological sample can be taken from, but not limited to the following fluids: organ perfusion fluid or organ perfusate (which can be near normothermic or hypothermic), peripheral blood, plasma, serum, ascites, cerebrospinal fluid (CSF), sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, nasal fluid (e.g., a nasal swab isolate) stool water, urine, pancreatic juice, lavage fluids from sinus cavities, bronchopulmonary aspirates or other lavage fluids. A biological sample may also include the blastocyst cavity, umbilical cord blood, or maternal circulation that may be of fetal or maternal origin. The biological sample may also be a tissue sample or biopsy or a cell population (e.g., a sample from a cell culture, such as a CAR T cell culture, pancreatic islet cell population, or stem cell population).

### Preparation of the Device for Hemopurification

The extracorporeal circuit is to be connected;
The extracorporeal circuit is primed and rinsed with a minimum of two liters of priming solution;
An anticoagulant such as heparin will be added per liter of priming solution if needed to prevent clotting of the blood circuit;
The initial flow rate for priming will be 200-250 mL/min and up to 400-500 ml/min for several minutes, while using a priming solution not blood, to increase the shear forces inside the fibers to encourage the dislodgement of microbubbles. During this procedure, all bubbles are removed from the tubing and the cartridge by gentle tapping.

### Therapy

For use on a subject with established vascular access, the subject will be connected to the dialysis machine, which pumps blood from the subject through the cartridge and returns the purified blood to the subject. Alternatively, an organ will be connected to a pump which will pump perfusate solution with or without red blood cells through the organ. Alternatively, an organ perfusate will be connected to a pump which will pump perfusate solution through the cartridge. Blood flow rates are typically maintained at up to 200 ml/min. In some embodiments, the flow rate of the blood or organ perfusate is 18 ml/min. In some embodiments, the flow rate of the blood or organ perfusate is 19 ml/min. Heparin injections are most often used to prevent blood clotting when the procedure is performed on a human. Typical treatment times are up to 4 hours. Longer times may be used to increase the effectiveness of the treatment. At the end of the treatment, the blood in the tubing and cartridge is washed back into the subject in the case of a treatment done on a human. The machine is then disconnected from the subject or the organ and the contaminated cartridge and blood tubing properly disposed.

During the therapy on person with the device, testing will be performed to measure the activated clotting times (ACT) for monitoring anticoagulation.

### Results

Pre- and post-treatment samples from a subject or an organ that will receive the therapy above will have a significantly reduced pathogens, viral particles, virus-like particles (VLPs), extracellular vesicles, such as exosomes or ectosomes, miRNAs, a biomarker or mediator of tissue damage, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, lipoglycans, or glycolipids, or fragments thereof in the blood post-therapy. When an organ, an organ perfusate, cell, or tissue transplantation procedure is accompanied by the therapy above, the organ, cell, or tissue viability, engraftment, and/or survival of the transplant and/or recipient is increased or improved as compared to the organ, cell, or tissue viability, engraftment, and/or survival of the transplant or recipient after transplantation in the absence of the therapy.

Samples collected from the subject at various intervals after therapy with a lectin affinity hemofiltration device will show reduced levels of pathogens, viral particles, virus-like particles (VLPs), extracellular vesicles, such as exosomes or ectosomes, inflammatory or immunomodulatory glycoproteins, lipoglycans, or glycolipids, or fragments thereof after treatment with the device vs. pre-treatment.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1—Preparation of GNA Covalently Coupled to Agarose Using Cyanogen Bromide

Cyanogen bromide (CNBr) activated agarose was used for direct coupling essentially according to Cuatrecasas, et al (Cuatracasas, Wilchek and Anfinsen. Proc Natl Acad Sci USA 61(2): 636-643, 1968). In brief, 1 ml of GNA at a concentration of 10 mg/ml in 0.1M NaHCO3 pH 9.5 is added to 1 ml CNBr activated agarose (Sigma, St. Louis, Mo.) and allowed to react overnight in the cold. Care must be taken to maintain alkaline pH to prevent the potential release of HCN gas. When the reaction is complete, unreacted materials are aspirated and the lectin coupled agarose washed extensively with sterile cold PBS. The lectin agarose affinity matrix is then stored cold until ready for use. Alternatively, GNA agarose is available commercially from Vector Labs (Burlingame, Calif.).

### Example 2—Preparation of an Exosome Specific Antibody or Lectin Covalently Coupled to Chromosorb (Diatomaceous Earth) Using Glutaraldehyde

Preparation of aminated diatomaceous earth is accomplished using γ-aminopropyl triethoxysilane (GAPS or APTES) (Sigma Chemical, St. Louis, Mo.) and Chromosorb 60/80 mesh. Although other grades of diatomaceous earth may be used, Chromosorb of this mesh size (200-300 microns in diameter) is often used to prevent small particulates from entering the sample through the largest available pore sizes found in hollow-fiber cartridges used for plasma separation (^{~}0.5 micron). Amino Chromosorb was prepared by suspension in an excess of 5% aqueous solution of GAPS in an overnight reaction. Aminated-Chromosorb was washed free of excess reagent with water and ethanol and dried overnight in a drying oven to yield an off white powder. One gram of the powder was then suspended in 5 ml 5% glutaraldehyde (Sigma) for 30 minutes. Excess glutaraldehyde was then removed by filtration and washing with water until no detectable aldehyde remained in the wash using Schiff's reagent (Sigma Chemical). The filter cake was then resuspended in 5 ml of Sigma borohydride coupling buffer containing 2-3 mg/ml of the antibody or lectin and the reaction allowed to proceed overnight at 4 degrees C. At the end of the reaction, excess antibody or lectin is washed off and the remaining aldehyde reacted with ethanolamine as described. After final washing in sterile PBS, the material was stored cold until ready for use.

### Example 3—Removal of Pathogens, Viral Particles, Virus-Like Particles (VLPs), Extracellular Vesicles, Such as Exosomes or Ectosomes, Nucleic Acids, Such as DNA or RNA, Circulating Extracellular DNA (ceDNA), Extracellular Vesicle Associated DNA, Neutrophil Extracellular Traps (NETs) and their Components Including DNA, Histones, Neutrophil Granule Proteins and/or Oxidant Enzymes, Inflammatory or Immunomodulatory Glycoproteins, Glycolipids or Lipoglycans, or Fragments Thereof in an Organ, Cell or a Tissue Donor Prior to Removal of the Organ, Cell or a Tissue

This example pertains to methods of removal of pathogens, viral particles, virus-like particles (VLPs), extracellular vesicles, such as exosomes or ectosomes, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans or fragments thereof from an organ, cell or a tissue donor prior to removal of the organ, a population of cells or a tissue from the donor. This example can be used in addition to performing examples 4 and 5 or individually.

Procedures for organ, cell or tissue donors prior to treatment with the lectin affinity hemofiltration device: Blood and/or plasma or organ perfusion solution or perfusate will be collected for pre-treatment assessment of pathogens, viral particles, virus-like particles (VLPs), extracellular vesicles, such as exosomes or ectosomes, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof. The results of the pre-treatment can be used for later comparison of pathogens, viral particles, virus-like particles (VLPs), extracellular vesicles, such as exosomes or ectosomes, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof present in the patient's circulatory system prior to treatment and in the post-treatment sample. Also, a pre-treatment blood and/or plasma or organ perfusion solution or perfusate sample may be used for detection of cytokines/inflammatory and other blood biomarkers of organ damage. In preparation for therapy, a hemodialysis catheter will be placed into the donor.

### Example 4—Removal of Pathogens, Viral Particles, Virus-Like Particles (VLPs), Extracellular Vesicles, Such as Exosomes or Ectosomes, Nucleic Acids, Such as DNA or RNA, Circulating Extracellular DNA (ceDNA), Extracellular Vesicle Associated DNA, Neutrophil Extracellular Traps (NETs) and their Components Including DNA, Histones, Neutrophil Granule Proteins and/or Oxidant Enzymes, Inflammatory or Immunomodulatory Glycoproteins, Glycolipids or Lipoglycans, or Fragments Thereof in a Perfused Organ

This example pertains to methods of removal of pathogens, viral particles, virus-like particles (VLPs), extracellular vesicles, such as exosomes or ectosomes, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof from a perfused organ and can be used in addition to performing examples 3 and 5 or individually.

Procedures for perfused organ prior to treatment with the lectin affinity hemofiltration device: The explanted donor organ will be connected directly to the Hemopurifier® filter. Typically, a pump will supply perfusion fluid via the artery supplying the organ. The perfusion fluid will then exit the organ via the vein and then enter the Hemopurifier® for removal of pathogens, viral particles, virus-like particles (VLPs), extracellular vesicles, such as exosomes or ectosomes, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof before being returned the artery supplying the organ. Perfusion buffer will be collected for pre-treatment assessment of pathogens, viral particles, virus-like particles (VLPs), extracellular vesicles, such as exosomes or ectosomes, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof. The results of the pre-treatment can be used for later comparison of pathogens, viral particles, virus-like particles (VLPs), extracellular vesicles, such as exosomes or ectosomes, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof present in the patient's circulatory system prior to treatment and in the post-treatment sample. Also, a pre-treatment perfusion buffer sample may be used for detection of cytokines/inflammatory and other blood biomarkers of organ damage.

### Example 5—Removal of Pathogens, Viral Particles, Virus-Like Particles (VLP), Extracellular Vesicles, Such as Exosomes or Ectosomes, Nucleic Acids, Such as DNA or RNA, Circulating Extracellular DNA (ceDNA), Extracellular Vesicle Associated DNA, Neutrophil Extracellular Traps (NETs) and their Components Including DNA, Histones, Neutrophil Granule Proteins and/or Oxidant Enzymes, Inflammatory or Immunomodulatory Glycoproteins, Glycolipids or Lipoglycans, or Fragments Thereof in a Transplant Recipient

This example pertains to methods of removal of pathogens, viral particles, virus-like particles (VLPs), extracellular vesicles, such as exosomes or ectosomes, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof in a transplant recipient. This example can be used in addition to performing examples 3 and 4 or individually.

Procedures for transplant recipient prior to treatment with the lectin affinity hemofiltration device: plasma or perfusate samples will be collected for pre-treatment assessment of pathogens, viral particles, virus-like particles (VLPs), extracellular vesicles, such as exosomes or ectosomes, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof. The results of the pre-treatment can be used for later comparison of pathogens, viral particles, virus-like particles (VLPs), extracellular vesicles, such as exosomes or ectosomes, nucleic acids, such as DNA or RNA, circulating extracellular DNA (ceDNA), extracellular vesicle associated DNA, Neutrophil extracellular traps (NETs) and their components including DNA, histones, neutrophil granule proteins and/or oxidant enzymes, inflammatory or immunomodulatory glycoproteins, glycolipids or lipoglycans, or fragments thereof present in the patient's circulatory system prior to treatment and in the post-treatment sample. Also, a pre-treatment blood and/or plasma sample may be used for detection of cytokines/inflammatory and other blood biomarkers of organ damage. In preparation for treatment, a hemodialysis catheter will be placed into the recipient.

### Examples 6: Removal of miRNAs from Kidney Perfusate

This example describes methods of removal of miRNAs or fragments thereof from a kidney perfusate and can be used in addition to performing examples disclosed herein or individually.

For this example, discarded kidneys were perfused using normothermic ex vivo machine perfusion. Following this, 250 milliliters of kidney perfusate was put into a circuit (FIG. 11 or FIG. 12) containing a full-scale Hemopurifier®, filled with 40 grams of GNA affinity resin. The perfusate was circulated at 200 milliliters per minute for a total of 24 total volume passes. RNA was then isolated using a Plasma/Serum RNA purification Mini Kit (Norgen Biotek Corp, Thorold, Canada) following the manufacturers protocol. In brief, 200 microliters of kidney perfusate samples were loaded onto spin columns with lysis buffer and ethanol. Samples were vortexed and mixed well. After vortexing, 650 μl of mixture was loaded onto a spin column and centrifuged at 400×g to 13,000×g based on the protocol. Retained RNA was washed to remove impurities. RNA was eluted with 20 μl of elution buffer. RNA was quantified using the Nanodrop One Spectrophotometer. At least 100 nanograms of total RNA from each sample was provided to either the Proof of Principle (PoP) Laboratory at NanoString (NanoString, Seattle, WA) or NanoString Services Laboratory at Canopy Biosciences (Canopy Biosciences, Hayward, CA). The samples were prepared for nCounter miRNA expression profiling according to the manufacturer's protocol. Before data normalization, Background Thresholding was performed. The threshold count value was set to 25 reads. Data normalization was performed using the geometric mean of the positive controls for code count normalization. Sample input amounts were normalized to the geometric mean of the positive ligation controls. The data was loaded into the R statistical environment (v4.3.1). Statistical analysis of experimental data was performed using R programing language (v4.3.2) and the integrated development environment RStudio. R and RStudio were used for data preprocessing, exploratory data analysis, hypothesis testing, and data visualization. A list of miRNAs depleted using devices as depicted in FIG. 11 or FIG. 12 are shown in Tables 1-4. Table 1 shows a list of miRNAs depleted using a normal flow device as depicted in FIG. 11. Table 2 shows a list of miRNAs depleted using a normal flow device as depicted in FIG. 11 with a p<0.1. Table 3 shows a list of miRNAs depleted using a side port flow device as depicted in FIG. 11. Table 4 shows a list of miRNAs depleted using a side port flow device as depicted in FIG. 11 with p<0.1.

TABLE 1
miRNAs Depleted using GNA Resin Normal Flow.

| | | GNA Resin - Normal Flow | | | |
|---|---|---|---|---|---|
| miRNA Name | Accession # | Fold Change | Log2 FC | Percent Change | p-value |
| hsa-let-7a-5p | MIMAT0000062 | 0.02 | −5.55 | −97.87 | 4.22E−03 |
| hsa-let-7b-5p | MIMAT0000063 | 0.44 | −1.18 | −55.86 | 0.20 |
| hsa-let-7c-5p | MIMAT0000064 | 0.28 | −1.86 | −72.45 | 0.12 |
| hsa-let-7d-5p | MIMAT0000065 | 0.35 | −1.51 | −64.89 | 0.15 |
| hsa-miR-29b-3p | MIMAT0000100 | 0.06 | −4.04 | −93.92 | 1.89E−02 |
| hsa-miR-99a-5p | MIMAT0000097 | 0.08 | −3.66 | −92.09 | 9.39E−03 |
| hsa-miR-15b-5p | MIMAT0000417 | 0.11 | −3.15 | −88.73 | 2.47E−02 |
| hsa-miR-1-3p | MIMAT0000416 | 0.35 | −1.52 | −65.13 | 0.57 |
| hsa-miR-548aa | MIMAT0018447 | 0.10 | −3.32 | −89.99 | 9.74E−03 |
| hsa-miR-106a-5p | MIMAT0000103 | 0.47 | −1.08 | −52.70 | 0.52 |
| hsa-miR-106b-5p | MIMAT0000680 | 0.56 | −0.84 | −44.14 | 0.44 |
| hsa-miR-10a-5p | MIMAT0000253 | 0.40 | −1.32 | −59.95 | 0.23 |
| hsa-miR-10b-5p | MIMAT0000254 | 0.32 | −1.63 | −67.69 | 0.14 |
| hsa-miR-1180-3p | MIMAT0005825 | 0.30 | −1.75 | −70.27 | 0.12 |
| hsa-miR-1183 | MIMAT0005828 | 0.57 | −0.81 | −42.96 | 0.43 |
| hsa-miR-122-5p | MIMAT0000421 | 0.93 | −0.11 | −7.34 | 0.92 |
| hsa-miR-1224-5p | MIMAT0005458 | 0.90 | −0.15 | −9.87 | 0.91 |
| hsa-miR-1246 | MIMAT0005898 | 0.66 | −0.61 | −34.48 | 0.60 |
| hsa-miR-1253 | MIMAT0005904 | 0.41 | −1.27 | −58.53 | 0.13 |
| hsa-miR-1258 | MIMAT0005909 | 0.95 | −0.07 | −4.74 | 0.90 |
| hsa-miR-125a-5p | MIMAT0000443 | 0.76 | −0.39 | −23.69 | 0.72 |
| hsa-miR-125b-5p | MIMAT0000423 | 0.40 | −1.32 | −59.95 | 0.15 |
| hsa-miR-100-5p | MIMAT0000098 | 0.24 | −2.04 | −75.68 | 0.10 |
| hsa-miR-548ah-5p | MIMAT0018972 | 0.81 | −0.3 | −18.77 | 0.65 |
| hsa-miR-98-5p | MIMAT0000096 | 0.68 | −0.56 | −32.17 | 0.66 |
| hsa-miR-4454 | MIMAT0018976 | 0.10 | −3.35 | −90.19 | 4.07E−02 |
| hsa-miR-128-3p | MIMAT0000424 | 0.66 | −0.61 | −34.48 | 0.56 |
| hsa-miR-1285-3p | MIMAT0005876 | 0.73 | −0.46 | −27.30 | 0.53 |
| hsa-miR-1285-5p | MIMAT0022719 | 0.59 | −0.76 | −40.95 | 0.12 |
| hsa-miR-1286 | MIMAT0005877 | 0.90 | −0.16 | −10.50 | 0.86 |
| hsa-miR-1268a | MIMAT0005922 | 0.85 | −0.23 | −14.74 | 0.88 |
| hsa-miR-1299 | MIMAT0005887 | 0.38 | −1.4 | −62.11 | 0.18 |
| hsa-miR-1306-5p | MIMAT0022726 | 0.87 | −0.2 | −12.94 | 0.70 |
| hsa-miR-1290 | MIMAT0005880 | 0.72 | −0.48 | −28.30 | 0.63 |
| hsa-miR-15a-5p | MIMAT0000068 | 0.12 | −3.05 | −87.93 | 3.33E−02 |
| hsa-miR-132-3p | MIMAT0000426 | 0.24 | −2.07 | −76.18 | 0.14 |
| hsa-miR-323a-3p | MIMAT0000755 | 0.77 | −0.37 | −22.62 | 0.53 |
| hsa-miR-133a-3p | MIMAT0000427 | 0.66 | −0.6 | −34.02 | 0.77 |
| hsa-miR-135a-5p | MIMAT0000428 | 0.46 | −1.12 | −53.99 | 0.44 |
| hsa-miR-135b-5p | MIMAT0000758 | 0.90 | −0.15 | −9.87 | 0.89 |
| hsa-miR-140-3p | MIMAT0004597 | 0.83 | −0.27 | −17.07 | 0.74 |
| hsa-miR-140-5p | MIMAT0000431 | 0.34 | −1.56 | −66.08 | 0.28 |
| hsa-miR-141-3p | MIMAT0000432 | 0.69 | −0.53 | −30.74 | 0.67 |
| hsa-miR-193a-5p | MIMAT0004614 | 0.13 | −2.91 | −86.70 | 4.95E−02 |
| hsa-miR-143-3p | MIMAT0000435 | 0.43 | −1.22 | −57.07 | 0.23 |
| hsa-miR-144-3p | MIMAT0000436 | 0.20 | −2.29 | −79.55 | 0.16 |
| hsa-miR-126-3p | MIMAT0000445 | 0.41 | −1.3 | −59.39 | 9.62E−02 |
| hsa-miR-199b-5p | MIMAT0000263 | 0.14 | −2.84 | −86.03 | 3.13E−02 |
| hsa-miR-142-3p | MIMAT0000434 | 0.15 | −2.7 | −84.61 | 3.36E−02 |
| hsa-miR-424-5p | MIMAT0001341 | 0.16 | −2.62 | −83.73 | 4.70E−02 |
| hsa-miR-150-5p | MIMAT0000451 | 0.54 | −0.88 | −45.66 | 0.28 |
| hsa-miR-151a-3p | MIMAT0000757 | 0.38 | −1.4 | −62.11 | 0.25 |
| hsa-miR-152-3p | MIMAT0000438 | 0.49 | −1.03 | −51.03 | 0.45 |
| hsa-miR-1537-3p | MIMAT0007399 | 0.76 | −0.39 | −23.69 | 0.54 |
| hsa-miR-154-5p | MIMAT0000452 | 0.56 | −0.83 | −43.75 | 0.42 |
| hsa-miR-1972 | MIMAT0009447 | 0.28 | −1.84 | −72.07 | 2.84E−02 |
| hsa-miR-361-5p | MIMAT0000703 | 0.16 | −2.6 | −83.51 | 9.25E−02 |
| hsa-miR-24-3p | MIMAT0000080 | 0.17 | −2.54 | −82.81 | 7.64E−02 |
| hsa-miR-16-5p | MIMAT0000069 | 0.35 | −1.53 | −65.37 | 0.13 |
| hsa-miR-181a-2-3p | MIMAT0004558 | 0.98 | −0.03 | −2.06 | 0.95 |
| hsa-miR-181a-5p | MIMAT0000256 | 0.33 | −1.61 | −67.24 | 0.24 |
| hsa-miR-181c-5p | MIMAT0000258 | 0.75 | −0.41 | −24.74 | 0.72 |
| hsa-miR-1827 | MIMAT0006767 | 0.97 | −0.05 | −3.41 | 0.92 |
| hsa-miR-185-5p | MIMAT0000455 | 0.59 | −0.77 | −41.36 | 0.52 |
| hsa-miR-186-5p | MIMAT0000456 | 0.51 | −0.96 | −48.59 | 0.45 |
| hsa-miR-187-3p | MIMAT0000262 | 0.81 | −0.31 | −19.34 | 0.55 |
| hsa-miR-4455 | MIMAT0018977 | 0.57 | −0.8 | −42.57 | 0.17 |
| hsa-miR-190b | MIMAT0004929 | 0.98 | −0.03 | −2.06 | 0.95 |
| hsa-miR-191-5p | MIMAT0000440 | 0.26 | −1.94 | −73.94 | 0.17 |
| hsa-miR-1910-5p | MIMAT0007884 | 0.44 | −1.2 | −56.47 | 0.31 |
| hsa-miR-1915-3p | MIMAT0007892 | 0.47 | −1.1 | −53.35 | 0.50 |
| hsa-miR-192-5p | MIMAT0000222 | 0.65 | −0.62 | −34.93 | 0.56 |
| hsa-miR-193a-3p | MIMAT0000459 | 0.75 | −0.42 | −25.26 | 0.78 |
| hsa-miR-23b-3p | MIMAT0000418 | 0.18 | −2.51 | −82.44 | 4.45E−02 |
| hsa-miR-193b-3p | MIMAT0002819 | 0.20 | −2.35 | −80.39 | 0.17 |
| hsa-miR-194-5p | MIMAT0000460 | 0.56 | −0.83 | −43.75 | 0.55 |
| hsa-miR-195-5p | MIMAT0000461 | 0.25 | −2 | −75.00 | 0.23 |
| hsa-miR-505-3p | MIMAT0002876 | 0.19 | −2.41 | −81.18 | 5.81E−02 |
| hsa-miR-574-3p | MIMAT0003239 | 0.19 | −2.41 | −81.18 | 9.02E−02 |
| hsa-miR-1976 | MIMAT0009451 | 0.88 | −0.18 | −11.73 | 0.70 |
| hsa-miR-198 | MIMAT0000228 | 0.90 | −0.15 | −9.87 | 0.76 |
| hsa-miR-199a-3p | MIMAT0000232 | 0.40 | −1.32 | −59.95 | 0.23 |
| hsa-miR-199a-5p | MIMAT0000231 | 0.49 | −1.02 | −50.69 | 0.29 |
| hsa-miR-30e-3p | MIMAT0000693 | 0.20 | −2.31 | −79.83 | 7.45E−02 |
| hsa-miR-19a-3p | MIMAT0000073 | 0.29 | −1.81 | −71.48 | 0.19 |
| hsa-miR-19b-3p | MIMAT0000074 | 0.30 | −1.75 | −70.27 | 0.18 |
| hsa-miR-200a-3p | MIMAT0000682 | 0.50 | −1.01 | −50.35 | 0.15 |
| hsa-miR-200b-3p | MIMAT0000318 | 0.57 | −0.82 | −43.36 | 0.15 |
| hsa-miR-203a-5p | MIMAT0031890 | 0.86 | −0.22 | −14.14 | 0.77 |
| hsa-miR-204-5p | MIMAT0000265 | 0.49 | −1.02 | −50.69 | 0.19 |
| hsa-miR-205-5p | MIMAT0000266 | 0.64 | −0.64 | −35.83 | 0.58 |
| hsa-miR-2053 | MIMAT0009978 | 0.93 | −0.1 | −6.70 | 0.87 |
| hsa-miR-206 | MIMAT0000462 | 0.73 | −0.45 | −26.80 | 0.88 |
| hsa-miR-208a-3p | MIMAT0000241 | 0.42 | −1.25 | −57.96 | 0.36 |
| hsa-miR-208b-3p | MIMAT0004960 | 0.83 | −0.27 | −17.07 | 0.86 |
| hsa-miR-20a-5p | MIMAT0000075 | 0.35 | −1.51 | −64.89 | 0.30 |
| hsa-miR-21-5p | MIMAT0000076 | 0.43 | −1.21 | −56.77 | 0.22 |
| hsa-miR-210-3p | MIMAT0000267 | 0.84 | −0.25 | −15.91 | 0.56 |
| hsa-miR-548ar-5p | MIMAT0022265 | 0.65 | −0.62 | −34.93 | 0.13 |
| hsa-miR-214-3p | MIMAT0000271 | 0.51 | −0.98 | −49.30 | 0.50 |
| hsa-miR-146a-5p | MIMAT0000449 | 0.22 | −2.21 | −78.39 | 9.51E−02 |
| hsa-miR-216b-5p | MIMAT0004959 | 0.75 | −0.42 | −25.26 | 0.62 |
| hsa-miR-335-5p | MIMAT0000765 | 0.22 | −2.18 | −77.93 | 7.86E−02 |
| hsa-miR-22-3p | MIMAT0000077 | 0.36 | −1.48 | −64.15 | 0.10 |
| hsa-miR-221-3p | MIMAT0000278 | 0.33 | −1.6 | −67.01 | 0.21 |
| hsa-miR-222-3p | MIMAT0000279 | 0.33 | −1.61 | −67.24 | 0.15 |
| hsa-miR-223-3p | MIMAT0000280 | 0.45 | −1.14 | −54.62 | 0.53 |
| hsa-miR-660-5p | MIMAT0003338 | 0.23 | −2.14 | −77.31 | 9.17E−02 |
| hsa-miR-377-3p | MIMAT0000730 | 0.23 | −2.1 | −76.67 | 6.02E−02 |
| hsa-miR-145-5p | MIMAT0000437 | 0.24 | −2.06 | −76.02 | 5.81E−02 |
| hsa-miR-218-5p | MIMAT0000275 | 0.24 | −2.06 | −76.02 | 9.56E−02 |
| hsa-miR-26a-5p | MIMAT0000082 | 0.36 | −1.47 | −63.90 | 0.40 |
| hsa-miR-26b-5p | MIMAT0000083 | 0.38 | −1.4 | −62.11 | 0.22 |
| hsa-miR-27a-3p | MIMAT0000084 | 0.73 | −0.45 | −26.80 | 0.52 |
| hsa-miR-27b-3p | MIMAT0000419 | 0.42 | −1.25 | −57.96 | 0.17 |
| hsa-miR-28-3p | MIMAT0004502 | 0.89 | −0.17 | −11.12 | 0.86 |
| hsa-miR-28-5p | MIMAT0000085 | 0.63 | −0.66 | −36.71 | 0.23 |
| hsa-miR-299-3p | MIMAT0000687 | 0.41 | −1.3 | −59.39 | 0.28 |
| hsa-miR-299-5p | MIMAT0002890 | 0.86 | −0.21 | −13.55 | 0.74 |
| hsa-miR-29a-3p | MIMAT0000086 | 0.26 | −1.97 | −74.47 | 0.13 |
| hsa-miR-495-3p | MIMAT0002817 | 0.24 | −2.06 | −76.02 | 1.48E−02 |
| hsa-miR-29c-3p | MIMAT0000681 | 0.44 | −1.19 | −56.17 | 0.25 |
| hsa-miR-301a-3p | MIMAT0000688 | 0.77 | −0.37 | −22.62 | 0.68 |
| hsa-miR-302b-3p | MIMAT0000715 | 0.63 | −0.66 | −36.71 | 0.19 |
| hsa-miR-30a-3p | MIMAT0000088 | 0.31 | −1.71 | −69.43 | 0.12 |
| hsa-let-7g-5p | MIMAT0000414 | 0.25 | −1.98 | −74.65 | 7.73E−02 |
| hsa-miR-30b-5p | MIMAT0000420 | 0.71 | −0.5 | −29.29 | 0.72 |
| hsa-miR-30c-5p | MIMAT0000244 | 0.90 | −0.16 | −10.50 | 0.83 |
| hsa-miR-127-3p | MIMAT0000446 | 0.27 | −1.89 | −73.02 | 4.66E−02 |
| hsa-miR-4286 | MIMAT0016916 | 0.66 | −0.59 | −33.57 | 0.39 |
| hsa-miR-30e-5p | MIMAT0000692 | 0.18 | −2.44 | −81.57 | 0.10 |
| hsa-miR-3158-3p | MIMAT0015032 | 0.50 | −1 | −50.00 | 0.58 |
| hsa-miR-3168 | MIMAT0015043 | 0.93 | −0.1 | −6.70 | 0.89 |
| hsa-miR-3180-5p | MIMAT0015057 | 0.93 | −0.1 | −6.70 | 0.84 |
| hsa-miR-3195 | MIMAT0015079 | 0.99 | −0.02 | −1.38 | 0.97 |
| hsa-miR-32-5p | MIMAT0000090 | 0.72 | −0.47 | −27.80 | 0.68 |
| hsa-miR-320a | MIMAT0000510 | 0.97 | −0.04 | −2.73 | 0.94 |
| hsa-miR-320e | MIMAT0015072 | 0.66 | −0.59 | −33.57 | 0.45 |
| hsa-let-7e-5p | MIMAT0000066 | 0.65 | −0.63 | −35.38 | 0.28 |
| hsa-miR-323b-3p | MIMAT0015050 | 0.80 | −0.32 | −19.89 | 0.70 |
| hsa-miR-324-5p | MIMAT0000761 | 0.23 | −2.14 | −77.31 | 0.13 |
| hsa-miR-325 | MIMAT0000771 | 0.99 | −0.01 | −0.69 | 0.99 |
| hsa-miR-148a-3p | MIMAT0000243 | 0.30 | −1.75 | −70.27 | 2.90E−02 |
| hsa-miR-337-3p | MIMAT0000754 | 0.99 | −0.02 | −1.38 | 0.98 |
| hsa-miR-337-5p | MIMAT0004695 | 0.61 | −0.71 | −38.87 | 0.58 |
| hsa-miR-33a-5p | MIMAT0000091 | 0.86 | −0.22 | −14.14 | 0.76 |
| hsa-miR-340-5p | MIMAT0004692 | 0.47 | −1.1 | −53.35 | 0.23 |
| hsa-miR-342-3p | MIMAT0000753 | 0.43 | −1.23 | −57.37 | 0.15 |
| hsa-miR-346 | MIMAT0000773 | 0.96 | −0.06 | −4.07 | 0.90 |
| hsa-miR-34a-5p | MIMAT0000255 | 0.44 | −1.17 | −55.56 | 0.15 |
| hsa-miR-361-3p | MIMAT0004682 | 0.32 | −1.63 | −67.69 | 0.19 |
| hsa-miR-497-5p | MIMAT0002820 | 0.30 | −1.73 | −69.85 | 6.24E−02 |
| hsa-miR-362-3p | MIMAT0004683 | 0.73 | −0.45 | −26.80 | 0.69 |
| hsa-miR-363-3p | MIMAT0000707 | 0.49 | −1.03 | −51.03 | 0.36 |
| hsa-miR-365a-3p | MIMAT0000710 | 0.35 | −1.51 | −64.89 | 0.18 |
| hsa-miR-664a-3p | MIMAT0005949 | 0.74 | −0.43 | −25.77 | 0.45 |
| hsa-miR-374a-5p | MIMAT0000727 | 0.18 | −2.45 | −81.70 | 0.11 |
| hsa-miR-376a-3p | MIMAT0000729 | 0.47 | −1.08 | −52.70 | 0.22 |
| hsa-miR-376c-3p | MIMAT0000720 | 0.37 | −1.45 | −63.40 | 0.32 |
| hsa-miR-216a-5p | MIMAT0000273 | 0.35 | −1.51 | −64.89 | 2.20E−02 |
| hsa-miR-378d | MIMAT0018926 | 0.42 | −1.26 | −58.25 | 0.15 |
| hsa-miR-378f | MIMAT0018932 | 0.59 | −0.75 | −40.54 | 0.28 |
| hsa-miR-378g | MIMAT0018937 | 0.34 | −1.55 | −65.85 | 0.14 |
| hsa-miR-378i | MIMAT0019074 | 0.45 | −1.14 | −54.62 | 0.10 |
| hsa-miR-379-5p | MIMAT0000733 | 0.92 | −0.12 | −7.98 | 0.80 |
| hsa-miR-381-3p | MIMAT0000736 | 0.78 | −0.36 | −22.08 | 0.70 |
| hsa-miR-382-5p | MIMAT0000737 | 0.31 | −1.69 | −69.01 | 0.16 |
| hsa-miR-421 | MIMAT0003339 | 0.41 | −1.27 | −58.53 | 0.28 |
| hsa-miR-423-3p | MIMAT0001340 | 0.39 | −1.36 | −61.04 | 0.13 |
| hsa-miR-423-5p | MIMAT0004748 | 0.37 | −1.42 | −62.63 | 0.23 |
| hsa-miR-130a-3p | MIMAT0000425 | 0.39 | −1.35 | −60.77 | 7.05E−02 |
| hsa-miR-425-5p | MIMAT0003393 | 0.93 | −0.11 | −7.34 | 0.90 |
| hsa-miR-190a-5p | MIMAT0000458 | 0.68 | −0.56 | −32.17 | 0.31 |
| hsa-miR-2117 | MIMAT0011162 | 0.71 | −0.5 | −29.29 | 0.34 |
| hsa-miR-548z | MIMAT0018446 | 0.69 | −0.54 | −31.22 | 0.29 |
| hsa-miR-25-3p | MIMAT0000081 | 0.40 | −1.34 | −60.50 | 2.77E−02 |
| hsa-miR-1323 | MIMAT0005795 | 0.72 | −0.47 | −27.80 | 0.39 |
| hsa-miR-450a-5p | MIMAT0001545 | 0.40 | −1.34 | −60.50 | 0.41 |
| hsa-miR-4516 | MIMAT0019053 | 0.86 | −0.22 | −14.14 | 0.84 |
| hsa-miR-451a | MIMAT0001631 | 0.68 | −0.56 | −32.17 | 0.45 |
| hsa-miR-487a-3p | MIMAT0002178 | 0.83 | −0.27 | −17.07 | 0.58 |
| hsa-miR-4532 | MIMAT0019071 | 0.41 | −1.3 | −59.39 | 0.41 |
| hsa-miR-455-5p | MIMAT0003150 | 0.47 | −1.1 | −53.35 | 0.40 |
| hsa-miR-483-5p | MIMAT0004761 | 0.97 | −0.04 | −2.73 | 0.94 |
| hsa-miR-486-3p | MIMAT0004762 | 0.48 | −1.07 | −52.37 | 0.30 |
| hsa-miR-4443 | MIMAT0018961 | 0.80 | −0.32 | −19.89 | 0.54 |
| hsa-miR-487b-3p | MIMAT0003180 | 0.92 | −0.12 | −7.98 | 0.81 |
| hsa-miR-491-5p | MIMAT0002807 | 0.66 | −0.6 | −34.02 | 0.54 |
| hsa-miR-543 | MIMAT0004954 | 0.40 | −1.32 | −59.95 | 5.04E−02 |
| hsa-miR-496 | MIMAT0002818 | 0.95 | −0.07 | −4.74 | 0.91 |
| hsa-let-7f-5p | MIMAT0000067 | 0.73 | −0.45 | −26.80 | 0.45 |
| hsa-miR-499a-5p | MIMAT0002870 | 0.60 | −0.73 | −39.71 | 0.69 |
| hsa-miR-503-5p | MIMAT0002874 | 0.45 | −1.14 | −54.62 | 0.34 |
| hsa-miR-148b-3p | MIMAT0000759 | 0.43 | −1.23 | −57.37 | 3.34E−02 |
| hsa-miR-506-3p | MIMAT0002878 | 0.65 | −0.62 | −34.93 | 0.52 |
| hsa-miR-509-3p | MIMAT0002881 | 0.33 | −1.58 | −66.55 | 0.14 |
| hsa-miR-509-5p | MIMAT0004779 | 0.90 | −0.16 | −10.50 | 0.74 |
| hsa-miR-513b-5p | MIMAT0005788 | 0.72 | −0.48 | −28.30 | 0.71 |
| hsa-miR-513c-5p | MIMAT0005789 | 0.80 | −0.33 | −20.45 | 0.75 |
| hsa-miR-514a-3p | MIMAT0002883 | 0.59 | −0.77 | −41.36 | 0.19 |
| hsa-miR-514a-5p | MIMAT0022702 | 0.72 | −0.48 | −28.30 | 0.65 |
| hsa-miR-514b-5p | MIMAT0015087 | 0.81 | −0.31 | −19.34 | 0.72 |
| hsa-miR-518b | MIMAT0002844 | 0.30 | −1.75 | −70.27 | 0.24 |
| hsa-miR-5196-3p | MIMAT0021129 | 0.95 | −0.08 | −5.39 | 0.87 |
| hsa-miR-520f-3p | MIMAT0002830 | 0.69 | −0.53 | −30.74 | 0.70 |
| hsa-miR-521 | MIMAT0002854 | 0.97 | −0.04 | −2.73 | 0.93 |
| hsa-miR-526a | MIMAT0002845 | 0.40 | −1.34 | −60.50 | 0.11 |
| hsa-miR-548d-5p | MIMAT0004812 | 0.74 | −0.43 | −25.77 | 0.41 |
| hsa-miR-532-5p | MIMAT0002888 | 0.96 | −0.06 | −4.07 | 0.89 |
| hsa-miR-30a-5p | MIMAT0000087 | 0.45 | −1.15 | −54.94 | 8.58E−02 |
| hsa-miR-30d-5p | MIMAT0000245 | 0.49 | −1.04 | −51.37 | 5.20E−02 |
| hsa-miR-196a-5p | MIMAT0000226 | 0.55 | −0.85 | −44.52 | 8.95E−02 |
| hsa-miR-136-5p | MIMAT0000448 | 0.79 | −0.34 | −21.00 | 0.48 |
| hsa-miR-4421 | MIMAT0018934 | 0.94 | −0.09 | −6.05 | 0.87 |
| hsa-miR-548m | MIMAT0005917 | 0.76 | −0.4 | −24.21 | 0.56 |
| hsa-miR-548n | MIMAT0005916 | 0.61 | −0.72 | −39.29 | 0.23 |
| hsa-miR-99b-5p | MIMAT0000689 | 0.85 | −0.23 | −14.74 | 0.64 |
| hsa-miR-551b-3p | MIMAT0003233 | 0.22 | −2.2 | −78.24 | 0.13 |
| hsa-miR-556-3p | MIMAT0004793 | 0.97 | −0.05 | −3.41 | 0.92 |
| hsa-miR-23a-3p | MIMAT0000078 | 0.52 | −0.95 | −48.24 | 9.64E−02 |
| hsa-miR-574-5p | MIMAT0004795 | 0.82 | −0.28 | −17.64 | 0.81 |
| hsa-miR-575 | MIMAT0003240 | 0.30 | −1.75 | −70.27 | 0.20 |
| hsa-miR-578 | MIMAT0003243 | 0.99 | −0.01 | −0.69 | 0.99 |
| hsa-miR-584-3p | MIMAT0022708 | 0.93 | −0.11 | −7.34 | 0.83 |
| hsa-miR-655-3p | MIMAT0003331 | 0.87 | −0.2 | −12.94 | 0.67 |
| hsa-miR-590-5p | MIMAT0003258 | 0.48 | −1.06 | −52.04 | 0.44 |
| hsa-miR-593-3p | MIMAT0004802 | 0.73 | −0.46 | −27.30 | 0.41 |
| hsa-miR-597-5p | MIMAT0003265 | 0.75 | −0.41 | −24.74 | 0.65 |
| hsa-miR-598-3p | MIMAT0003266 | 0.67 | −0.57 | −32.64 | 0.60 |
| hsa-miR-601 | MIMAT0003269 | 0.78 | −0.35 | −21.54 | 0.74 |
| hsa-miR-607 | MIMAT0003275 | 0.69 | −0.53 | −30.74 | 0.42 |
| hsa-miR-616-3p | MIMAT0004805 | 0.61 | −0.71 | −38.87 | 0.48 |
| hsa-miR-625-5p | MIMAT0003294 | 0.90 | −0.15 | −9.87 | 0.74 |
| hsa-miR-630 | MIMAT0003299 | 0.57 | −0.81 | −42.96 | 0.57 |
| hsa-miR-656-3p | MIMAT0003332 | 0.70 | −0.52 | −30.26 | 0.57 |
| hsa-miR-663a | MIMAT0003326 | 0.70 | −0.51 | −29.78 | 0.72 |
| hsa-miR-7-5p | MIMAT0000252 | 0.65 | −0.62 | −34.93 | 0.33 |
| hsa-miR-764 | MIMAT0010367 | 0.95 | −0.07 | −4.74 | 0.92 |
| hsa-miR-769-5p | MIMAT0003886 | 0.80 | −0.32 | −19.89 | 0.73 |
| hsa-miR-873-5p | MIMAT0004953 | 0.92 | −0.12 | −7.98 | 0.79 |
| hsa-miR-887-3p | MIMAT0004951 | 0.87 | −0.2 | −12.94 | 0.72 |
| hsa-miR-888-5p | MIMAT0004916 | 0.68 | −0.56 | −32.17 | 0.46 |
| hsa-miR-93-5p | MIMAT0000093 | 0.37 | −1.43 | −62.89 | 0.26 |
| hsa-miR-95-3p | MIMAT0000094 | 0.30 | −1.73 | −69.85 | 0.20 |

TABLE 2
miRNAs Significantly Depleted using GNA Normal Flow.

| | | GNA Resin - Normal Flow | | | |
|---|---|---|---|---|---|
| miRNA Name | Accession # | Fold Change | Log2 FC | Percent Change | p-value |
| hsa-let-7a-5p | MIMAT0000062 | 0.02 | −5.55 | −97.87 | 4.22E−03 |
| hsa-miR-29b-3p | MIMAT0000100 | 0.06 | −4.04 | −93.92 | 1.89E−02 |
| hsa-miR-99a-5p | MIMAT0000097 | 0.08 | −3.66 | −92.09 | 9.39E−03 |
| hsa-miR-15b-5p | MIMAT0000417 | 0.11 | −3.15 | −88.73 | 2.47E−02 |
| hsa-miR-548aa | MIMAT0018447 | 0.10 | −3.32 | −89.99 | 9.74E−03 |
| hsa-miR-4454 | MIMAT0018976 | 0.10 | −3.35 | −90.19 | 4.07E−02 |
| hsa-miR-15a-5p | MIMAT0000068 | 0.12 | −3.05 | −87.93 | 3.33E−02 |
| hsa-miR-193a-5p | MIMAT0004614 | 0.13 | −2.91 | −86.70 | 4.95E−02 |
| hsa-miR-126-3p | MIMAT0000445 | 0.41 | −1.3 | −59.39 | 9.62E−02 |
| hsa-miR-199b-5p | MIMAT0000263 | 0.14 | −2.84 | −86.03 | 3.13E−02 |
| hsa-miR-142-3p | MIMAT0000434 | 0.15 | −2.3 | −84.61 | 3.36E−02 |
| hsa-miR-424-5p | MIMAT0001341 | 0.16 | −2.62 | −83.73 | 4.70E−02 |
| hsa-miR-1972 | MIMAT0009447 | 0.28 | −1.84 | −72.07 | 2.84E−02 |
| hsa-miR-361-5p | MIMAT0000703 | 0.16 | −2.6 | −83.51 | 9.25E−02 |
| hsa-miR-24-3p | MIMAT0000080 | 0.17 | −2.54 | −82.81 | 7.64E−02 |
| hsa-miR-23b-3p | MIMAT0000418 | 0.18 | −2.51 | −82.44 | 4.45E−02 |
| hsa-miR-505-3p | MIMAT0002876 | 0.19 | −2.41 | −81.18 | 5.81E−02 |
| hsa-miR-574-3p | MIMAT0003239 | 0.19 | −2.41 | −81.18 | 9.02E−02 |
| hsa-miR-30e-3p | MIMAT0000693 | 0.20 | −2.31 | −79.83 | 7.45E−02 |
| hsa-miR-146a-5p | MIMAT0000449 | 0.22 | −2.21 | −78.39 | 9.51E−02 |
| hsa-miR-335-5p | MIMAT0000765 | 0.22 | −2.18 | −77.93 | 7.86E−02 |
| hsa-miR-660-5p | MIMAT0003338 | 0.23 | −2.14 | −77.31 | 9.17E−02 |
| hsa-miR-377-3p | MIMAT0000730 | 0.23 | −2.1 | −76.67 | 6.02E−02 |
| hsa-miR-145-5p | MIMAT0000437 | 0.24 | −2.06 | −76.02 | 5.81E−02 |
| hsa-miR-218-5p | MIMAT0000275 | 0.24 | −2.06 | −76.02 | 9.56E−02 |
| hsa-miR-495-3p | MIMAT0002817 | 0.24 | −2.06 | −76.02 | 1.48E−02 |
| hsa-let-7g-5p | MIMAT0000414 | 0.25 | −1.98 | −74.65 | 7.73E−02 |
| hsa-miR-127-3p | MIMAT0000446 | 0.27 | −1.89 | −73.02 | 4.66E−02 |
| hsa-miR-148a-3p | MIMAT0000243 | 0.30 | −1.75 | −70.27 | 2.90E−02 |
| hsa-miR-497-5p | MIMAT0002820 | 0.30 | −1.73 | −69.85 | 6.24E−02 |
| hsa-miR-216a-5p | MIMAT0000273 | 0.35 | −1.51 | −64.89 | 2.20E−02 |
| hsa-miR-130a-3p | MIMAT0000425 | 0.39 | −1.35 | −60.77 | 7.05E−02 |
| hsa-miR-25-3p | MIMAT0000081 | 0.40 | −1.34 | −60.50 | 2.77E−02 |
| hsa-miR-543 | MIMAT0004954 | 0.40 | −1.32 | −59.95 | 5.04E−02 |
| hsa-miR-148b-3p | MIMAT0000759 | 0.43 | −1.23 | −57.37 | 3.34E−02 |
| hsa-miR-30a-5p | MIMAT0000087 | 0.45 | −1.15 | −54.94 | 8.58E−02 |
| hsa-miR-30d-5p | MIMAT0000245 | 0.49 | −1.04 | −51.37 | 5.20E−02 |
| hsa-miR-196a-5p | MIMAT0000226 | 0.55 | −0.85 | −44.52 | 8.95E−02 |
| hsa-miR-23a-3p | MIMAT0000078 | 0.52 | −0.95 | −48.24 | 9.64E−02 |

TABLE 3
miRNAs Depleted using GNA Resin Side Port Flow.

| | | GNA Resin - Side Port Flow | | | |
|---|---|---|---|---|---|
| miRNA Name | Accession # | Fold Change | Log2 FC | Percent Change | p-value |
| hsa-let-7a-5p | MIMAT0000062 | 0.025 | −5.32 | −97.50 | 2.68E−02 |
| hsa-let-7b-5p | MIMAT0000063 | 0.423 | −1.24 | −57.66 | 0.27 |
| hsa-let-7c-5p | MIMAT0000064 | 0.291 | −1.78 | −70.88 | 0.22 |
| hsa-let-7d-5p | MIMAT0000065 | 0.727 | −0.46 | −27.30 | 0.68 |
| hsa-miR-29b-3p | MIMAT0000100 | 0.047 | −4.42 | −95.33 | 5.62E−02 |
| hsa-miR-99a-5p | MIMAT0000097 | 0.075 | −3.74 | −92.52 | 2.86E−02 |
| hsa-miR-15b-5p | MIMAT0000417 | 0.092 | −3.45 | −90.85 | 7.16E−02 |
| hsa-let-7i-5p | MIMAT0000415 | 0.476 | −1.07 | −52.37 | 0.28 |
| hsa-miR-1-3p | MIMAT0000416 | 0.590 | −0.76 | −40.95 | 0.81 |
| hsa-miR-1-5p | MIMAT0031892 | 0.642 | −0.64 | −35.83 | 0.25 |
| hsa-miR-548aa | MIMAT0018447 | 0.098 | −3.35 | −90.19 | 3.80E−02 |
| hsa-miR-101-3p | MIMAT0000099 | 0.812 | −0.3 | −18.77 | 0.26 |
| hsa-miR-103a-3p | MIMAT0000101 | 0.927 | −0.11 | −7.34 | 0.62 |
| hsa-miR-106a-5p | MIMAT0000103 | 0.540 | −0.89 | −46.04 | 0.61 |
| hsa-miR-106b-5p | MIMAT0000680 | 0.429 | −1.22 | −57.07 | 0.30 |
| hsa-miR-10a-5p | MIMAT0000253 | 0.599 | −0.74 | −40.13 | 0.44 |
| hsa-miR-10b-5p | MIMAT0000254 | 0.366 | −1.45 | −63.40 | 0.18 |
| hsa-miR-1178-3p | MIMAT0005823 | 0.927 | −0.11 | −7.34 | 0.62 |
| hsa-miR-1180-3p | MIMAT0005825 | 0.409 | −1.29 | −59.10 | 0.36 |
| hsa-miR-1183 | MIMAT0005828 | 0.768 | −0.38 | −23.16 | 0.76 |
| hsa-miR-1185-1-3p | MIMAT0022838 | 0.927 | −0.11 | −7.34 | 0.62 |
| hsa-miR-1185-2-3p | MIMAT0022713 | 0.847 | −0.24 | −15.33 | 0.40 |
| hsa-miR-1193 | MIMAT0015049 | 0.927 | −0.11 | −7.34 | 0.62 |
| hsa-miR-1200 | MIMAT0005863 | 0.927 | −0.11 | −7.34 | 0.62 |
| hsa-miR-1202 | MIMAT0005865 | 0.927 | −0.11 | −7.34 | 0.62 |
| hsa-miR-1203 | MIMAT0005866 | 0.927 | −0.11 | −7.34 | 0.62 |
| hsa-miR-1204 | MIMAT0005868 | 0.927 | −0.11 | −7.34 | 0.62 |
| hsa-miR-1205 | MIMAT0005869 | 0.927 | −0.11 | −7.34 | 0.62 |
| hsa-miR-1206 | MIMAT0005870 | 0.927 | −0.11 | −7.34 | 0.62 |
| hsa-miR-122-5p | MIMAT0000421 | 0.979 | −0.03 | −2.06 | 0.98 |
| hsa-miR-1226-3p | MIMAT0005577 | 0.927 | −0.11 | −7.34 | 0.62 |
| hsa-miR-1228-3p | MIMAT0005583 | 0.847 | −0.24 | −15.33 | 0.40 |
| hsa-miR-1233-3p | MIMAT0005588 | 0.927 | −0.11 | −7.34 | 0.62 |
| hsa-miR-1234-3p | MIMAT0005589 | 0.927 | −0.11 | −7.34 | 0.62 |
| hsa-miR-1236-3p | MIMAT0005591 | 0.927 | −0.11 | −7.34 | 0.62 |
| hsa-miR-124-3p | MIMAT0000422 | 0.927 | −0.11 | −7.34 | 0.62 |
| hsa-miR-1244 | MIMAT0005896 | 0.927 | −0.11 | −7.34 | 0.62 |
| hsa-miR-1245a | MIMAT0005897 | 0.927 | −0.11 | −7.34 | 0.62 |
| hsa-miR-1245b-3p | MIMAT0019951 | 0.927 | −0.11 | −7.34 | 0.62 |
| hsa-miR-1245b-5p | MIMAT0019950 | 0.927 | −0.11 | −7.34 | 0.62 |
| hsa-miR-1246 | MIMAT0005898 | 0.732 | −0.45 | −26.80 | 0.38 |
| hsa-miR-1247-5p | MIMAT0005899 | 0.927 | −0.11 | −7.34 | 0.62 |
| hsa-miR-1248 | MIMAT0005900 | 0.927 | −0.11 | −7.34 | 0.62 |
| hsa-miR-1249-3p | MIMAT0005901 | 0.927 | −0.11 | −7.34 | 0.62 |
| hsa-miR-1249-5p | MIMAT0032029 | 0.927 | −0.11 | −7.34 | 0.62 |
| hsa-miR-1250-5p | MIMAT0005902 | 0.927 | −0.11 | −7.34 | 0.62 |
| hsa-miR-1252-5p | MIMAT0005944 | 0.927 | −0.11 | −7.34 | 0.62 |
| hsa-miR-1253 | MIMAT0005904 | 0.525 | −0.93 | −47.51 | 0.33 |
| hsa-miR-1254 | MIMAT0005905 | 0.927 | −0.11 | −7.34 | 0.62 |
| hsa-miR-1255a | MIMAT0005906 | 0.979 | −0.03 | −2.06 | 0.98 |
| hsa-miR-1255b-5p | MIMAT0005945 | 0.758 | −0.4 | −24.21 | 0.23 |
| hsa-miR-1257 | MIMAT0005908 | 0.908 | −0.14 | −9.25 | 0.77 |
| hsa-miR-125a-5p | MIMAT0000443 | 0.712 | −0.49 | −28.80 | 0.72 |
| hsa-miR-125b-5p | MIMAT0000423 | 0.426 | −1.23 | −57.37 | 0.26 |
| hsa-miR-100-5p | MIMAT0000098 | 0.136 | −2.88 | −86.42 | 8.40E−02 |
| hsa-miR-1260a | MIMAT0005911 | 0.927 | −0.11 | −7.34 | 0.62 |
| hsa-miR-1260b | MIMAT0015041 | 0.927 | −0.11 | −7.34 | 0.62 |
| hsa-miR-1261 | MIMAT0005913 | 0.927 | −0.11 | −7.34 | 0.62 |
| hsa-miR-1262 | MIMAT0005914 | 0.927 | −0.11 | −7.34 | 0.62 |
| hsa-miR-1264 | MIMAT0005791 | 0.664 | −0.59 | −33.57 | 0.12 |
| hsa-miR-1266-5p | MIMAT0005920 | 0.927 | −0.11 | −7.34 | 0.62 |
| hsa-miR-548ah-5p | MIMAT0018972 | 0.245 | −2.03 | −75.51 | 4.44E−03 |
| hsa-miR-1268b | MIMAT0018925 | 0.927 | −0.11 | −7.34 | 0.62 |
| hsa-miR-98-5p | MIMAT0000096 | 0.255 | −1.97 | −74.47 | 5.36E−02 |
| hsa-miR-1269b | MIMAT0019059 | 0.927 | −0.11 | −7.34 | 0.62 |
| hsa-miR-4454 | MIMAT0018976 | 0.136 | −2.88 | −86.42 | 0.16 |
| hsa-miR-127-5p | MIMAT0004604 | 0.927 | −0.11 | −7.34 | 0.62 |
| hsa-miR-1270 | MIMAT0005924 | 0.927 | −0.11 | −7.34 | 0.62 |
| hsa-miR-1271-3p | MIMAT0022712 | 0.927 | −0.11 | −7.34 | 0.62 |
| hsa-miR-1271-5p | MIMAT0005796 | 0.927 | −0.11 | −7.34 | 0.62 |
| hsa-miR-1272 | MIMAT0005925 | 0.927 | −0.11 | −7.34 | 0.62 |
| hsa-miR-1273c | MIMAT0015017 | 0.927 | −0.11 | −7.34 | 0.62 |
| hsa-miR-1275 | MIMAT0005929 | 0.927 | −0.11 | −7.34 | 0.62 |
| hsa-miR-1276 | MIMAT0005930 | 0.927 | −0.11 | −7.34 | 0.62 |
| hsa-miR-1277-3p | MIMAT0005933 | 0.927 | −0.11 | −7.34 | 0.62 |
| hsa-miR-1278 | MIMAT0005936 | 0.927 | −0.11 | −7.34 | 0.62 |
| hsa-miR-1279 | MIMAT0005937 | 0.927 | −0.11 | −7.34 | 0.62 |
| hsa-miR-128-1-5p | MIMAT0026477 | 0.927 | −0.11 | −7.34 | 0.62 |
| hsa-miR-128-2-5p | MIMAT0031095 | 0.927 | −0.11 | −7.34 | 0.62 |
| hsa-miR-128-3p | MIMAT0000424 | 0.507 | −0.98 | −49.30 | 0.37 |
| hsa-miR-1281 | MIMAT0005939 | 0.927 | −0.11 | −7.34 | 0.62 |
| hsa-miR-1283 | MIMAT0005799 | 0.927 | −0.11 | −7.34 | 0.62 |
| hsa-miR-1285-5p | MIMAT0022719 | 0.655 | −0.61 | −34.48 | 0.15 |
| hsa-miR-1287-3p | MIMAT0026738 | 0.927 | −0.11 | −7.34 | 0.62 |
| hsa-miR-1287-5p | MIMAT0005878 | 0.758 | −0.4 | −24.21 | 0.12 |
| hsa-miR-1288-3p | MIMAT0005942 | 0.927 | −0.11 | −7.34 | 0.62 |
| hsa-miR-1289 | MIMAT0005879 | 0.927 | −0.11 | −7.34 | 0.62 |
| hsa-miR-129-2-3p | MIMAT0004605 | 0.927 | −0.11 | −7.34 | 0.62 |
| hsa-miR-129-5p | MIMAT0000242 | 0.927 | −0.11 | −7.34 | 0.62 |
| hsa-miR-1268a | MIMAT0005922 | 0.279 | −1.84 | −72.07 | 7.22E−02 |
| hsa-miR-1291 | MIMAT0005881 | 0.927 | −0.11 | −7.34 | 0.62 |
| hsa-miR-1293 | MIMAT0005883 | 0.927 | −0.11 | −7.34 | 0.62 |
| hsa-miR-1296-5p | MIMAT0005794 | 0.927 | −0.11 | −7.34 | 0.62 |
| hsa-miR-1297 | MIMAT0005886 | 0.859 | −0.22 | −14.14 | 0.33 |
| hsa-miR-1298-5p | MIMAT0005800 | 0.758 | −0.4 | −24.21 | 0.33 |
| hsa-miR-1299 | MIMAT0005887 | 0.871 | −0.2 | −12.94 | 0.88 |
| hsa-miR-1301-3p | MIMAT0005797 | 0.927 | −0.11 | −7.34 | 0.62 |
| hsa-miR-1302 | MIMAT0005890 | 0.927 | −0.11 | −7.34 | 0.62 |
| hsa-miR-1303 | MIMAT0005891 | 0.927 | −0.11 | −7.34 | 0.62 |
| hsa-miR-1304-3p | MIMAT0022720 | 0.927 | −0.11 | −7.34 | 0.62 |
| hsa-miR-1304-5p | MIMAT0005892 | 0.979 | −0.03 | −2.06 | 0.90 |
| hsa-miR-1305 | MIMAT0005893 | 0.877 | −0.19 | −12.34 | 0.85 |
| hsa-miR-1306-3p | MIMAT0005950 | 0.927 | −0.11 | −7.34 | 0.62 |
| hsa-miR-1306-5p | MIMAT0022726 | 0.646 | −0.63 | −35.38 | 0.12 |
| hsa-miR-1290 | MIMAT0005880 | 0.301 | −1.73 | −69.85 | 5.08E−02 |
| hsa-miR-15a-5p | MIMAT0000068 | 0.321 | −1.64 | −67.91 | 9.03E−02 |
| hsa-miR-130b-3p | MIMAT0000691 | 0.927 | −0.11 | −7.34 | 0.62 |
| hsa-miR-132-3p | MIMAT0000426 | 0.480 | −1.06 | −52.04 | 0.52 |
| hsa-miR-1322 | MIMAT0005953 | 0.927 | −0.11 | −7.34 | 0.62 |
| hsa-miR-323a-3p | MIMAT0000755 | 0.344 | −1.54 | −65.61 | 5.50E−03 |
| hsa-miR-133a-5p | MIMAT0026478 | 0.927 | −0.11 | −7.34 | 0.62 |
| hsa-miR-133b | MIMAT0000770 | 0.927 | −0.11 | −7.34 | 0.62 |
| hsa-miR-134-3p | MIMAT0026481 | 0.927 | −0.11 | −7.34 | 0.62 |
| hsa-miR-134-5p | MIMAT0000447 | 0.927 | −0.11 | −7.34 | 0.62 |
| hsa-miR-135a-5p | MIMAT0000428 | 0.351 | −1.51 | −64.89 | 0.48 |
| hsa-miR-135b-5p | MIMAT0000758 | 0.763 | −0.39 | −23.69 | 0.77 |
| hsa-miR-369-3p | MIMAT0000721 | 0.379 | −1.4 | −62.11 | 3.90E−02 |
| hsa-miR-137 | MIMAT0000429 | 0.927 | −0.11 | −7.34 | 0.62 |
| hsa-miR-138-5p | MIMAT0000430 | 0.993 | −0.01 | −0.69 | 0.96 |
| hsa-miR-139-3p | MIMAT0004552 | 0.871 | −0.2 | −12.94 | 0.52 |
| hsa-miR-139-5p | MIMAT0000250 | 0.927 | −0.11 | −7.34 | 0.62 |
| hsa-miR-140-3p | MIMAT0004597 | 0.620 | −0.69 | −38.01 | 0.31 |
| hsa-miR-140-5p | MIMAT0000431 | 0.287 | −1.8 | −71.28 | 0.41 |
| hsa-miR-141-3p | MIMAT0000432 | 0.599 | −0.74 | −40.13 | 0.63 |
| hsa-miR-193a-5p | MIMAT0004614 | 0.390 | −1.36 | −61.04 | 9.22E−02 |
| hsa-miR-142-5p | MIMAT0000433 | 0.927 | −0.11 | −7.34 | 0.62 |
| hsa-miR-143-3p | MIMAT0000435 | 0.547 | −0.87 | −45.29 | 0.47 |
| hsa-miR-144-3p | MIMAT0000436 | 0.521 | −0.94 | −47.88 | 0.61 |
| hsa-miR-126-3p | MIMAT0000445 | 0.392 | −1.35 | −60.77 | 7.95E−02 |
| hsa-miR-1469 | MIMAT0007347 | 0.927 | −0.11 | −7.34 | 0.62 |
| hsa-miR-199b-5p | MIMAT0000263 | 0.293 | −1.77 | −70.68 | 0.23 |
| hsa-miR-146b-3p | MIMAT0004766 | 0.927 | −0.11 | −7.34 | 0.62 |
| hsa-miR-147a | MIMAT0000251 | 0.927 | −0.11 | −7.34 | 0.62 |
| hsa-miR-147b | MIMAT0004928 | 0.927 | −0.11 | −7.34 | 0.62 |
| hsa-miR-142-3p | MIMAT0000434 | 0.212 | −2.24 | −78.83 | 0.10 |
| hsa-miR-424-5p | MIMAT0001341 | 0.540 | −0.89 | −46.04 | 0.27 |
| hsa-miR-149-5p | MIMAT0000450 | 0.927 | −0.11 | −7.34 | 0.62 |
| hsa-miR-150-5p | MIMAT0000451 | 0.438 | −1.19 | −56.17 | 0.25 |
| hsa-miR-151a-3p | MIMAT0000757 | 0.401 | −1.32 | −59.95 | 0.48 |
| hsa-miR-151a-5p | MIMAT0004697 | 0.503 | −0.99 | −49.65 | 0.13 |
| hsa-miR-151b | MIMAT0010214 | 0.895 | −0.16 | −10.50 | 0.45 |
| hsa-miR-152-3p | MIMAT0000438 | 0.460 | −1.12 | −53.99 | 0.50 |
| hsa-miR-152-5p | MIMAT0026479 | 0.927 | −0.11 | −7.34 | 0.62 |
| hsa-miR-1537-3p | MIMAT0007399 | 0.633 | −0.66 | −36.71 | 0.34 |
| hsa-miR-154-5p | MIMAT0000452 | 0.429 | −1.22 | −57.07 | 0.26 |
| hsa-miR-1972 | MIMAT0009447 | 0.403 | −1.31 | −59.67 | 2.95E−03 |
| hsa-miR-361-5p | MIMAT0000703 | 0.563 | −0.83 | −43.75 | 0.46 |
| hsa-miR-24-3p | MIMAT0000080 | 0.202 | −2.31 | −79.83 | 0.26 |
| hsa-miR-16-5p | MIMAT0000069 | 0.457 | −1.13 | −54.31 | 0.31 |
| hsa-miR-181a-2-3p | MIMAT0004558 | 0.697 | −0.52 | −30.26 | 0.15 |
| hsa-miR-181a-5p | MIMAT0000256 | 0.198 | −2.34 | −80.25 | 0.29 |
| hsa-miR-181b-2-3p | MIMAT0031893 | 0.678 | −0.56 | −32.17 | 0.13 |
| hsa-miR-181b-5p | MIMAT0000257 | 0.742 | −0.43 | −25.77 | 0.66 |
| hsa-miR-181c-5p | MIMAT0000258 | 0.790 | −0.34 | −21.00 | 0.83 |
| hsa-miR-181d-3p | MIMAT0026608 | 0.927 | −0.11 | −7.34 | 0.62 |
| hsa-miR-182-3p | MIMAT0000260 | 0.927 | −0.11 | −7.34 | 0.62 |
| hsa-miR-182-5p | MIMAT0000259 | 0.927 | −0.11 | −7.34 | 0.62 |
| hsa-miR-1827 | MIMAT0006767 | 0.688 | −0.54 | −31.22 | 0.16 |
| hsa-miR-183-5p | MIMAT0000261 | 0.841 | −0.25 | −15.91 | 0.84 |
| hsa-miR-184 | MIMAT0000454 | 0.889 | −0.17 | −11.12 | 0.45 |
| hsa-miR-186-5p | MIMAT0000456 | 0.953 | −0.07 | −4.74 | 0.96 |
| hsa-miR-187-3p | MIMAT0000262 | 0.889 | −0.17 | −11.12 | 0.82 |
| hsa-miR-188-3p | MIMAT0004613 | 0.927 | −0.11 | −7.34 | 0.62 |
| hsa-miR-18a-5p | MIMAT0000072 | 0.914 | −0.13 | −8.62 | 0.52 |
| hsa-miR-18b-5p | MIMAT0001412 | 0.818 | −0.29 | −18.21 | 0.26 |
| hsa-miR-1908-3p | MIMAT0026916 | 0.927 | −0.11 | −7.34 | 0.62 |
| hsa-miR-1908-5p | MIMAT0007881 | 0.927 | −0.11 | −7.34 | 0.62 |
| hsa-miR-1909-3p | MIMAT0007883 | 0.927 | −0.11 | −7.34 | 0.62 |
| hsa-miR-190a-3p | MIMAT0026482 | 0.642 | −0.64 | −35.83 | 0.25 |
| hsa-miR-4455 | MIMAT0018977 | 0.426 | −1.23 | −57.37 | 1.84E−02 |
| hsa-miR-190b | MIMAT0004929 | 0.727 | −0.46 | −27.30 | 0.23 |
| hsa-miR-191-5p | MIMAT0000440 | 0.514 | −0.96 | −48.59 | 0.44 |
| hsa-miR-1910-3p | MIMAT0026917 | 0.927 | −0.11 | −7.34 | 0.62 |
| hsa-miR-1910-5p | MIMAT0007884 | 0.616 | −0.7 | −38.44 | 0.69 |
| hsa-miR-1915-3p | MIMAT0007892 | 0.547 | −0.87 | −45.29 | 0.65 |
| hsa-miR-193a-3p | MIMAT0000459 | 0.551 | −0.86 | −44.90 | 0.58 |
| hsa-miR-23b-3p | MIMAT0000418 | 0.480 | −1.06 | −52.04 | 0.31 |
| hsa-miR-193b-3p | MIMAT0002819 | 0.268 | −1.9 | −73.21 | 0.37 |
| hsa-miR-194-5p | MIMAT0000460 | 0.268 | −1.9 | −73.21 | 0.24 |
| hsa-miR-195-5p | MIMAT0000461 | 0.346 | −1.53 | −65.37 | 0.47 |
| hsa-miR-196a-3p | MIMAT0004562 | 0.927 | −0.11 | −7.34 | 0.62 |
| hsa-miR-505-3p | MIMAT0002876 | 0.403 | −1.31 | −59.67 | 0.46 |
| hsa-miR-196b-5p | MIMAT0001080 | 0.927 | −0.11 | −7.34 | 0.62 |
| hsa-miR-574-3p | MIMAT0003239 | 0.291 | −1.78 | −70.88 | 0.35 |
| hsa-miR-1973 | MIMAT0009448 | 0.812 | −0.3 | −18.77 | 0.37 |
| hsa-miR-1976 | MIMAT0009451 | 0.953 | −0.07 | −4.74 | 0.91 |
| hsa-miR-199a-3p | MIMAT0000232 | 0.470 | −1.09 | −53.02 | 0.53 |
| hsa-miR-199a-5p | MIMAT0000231 | 0.624 | −0.68 | −37.58 | 0.55 |
| hsa-miR-30e-3p | MIMAT0000693 | 0.291 | −1.78 | −70.88 | 0.30 |
| hsa-miR-19a-3p | MIMAT0000073 | 0.423 | −1.24 | −57.66 | 0.50 |
| hsa-miR-19b-3p | MIMAT0000074 | 0.503 | −0.99 | −49.65 | 0.57 |
| hsa-miR-200a-3p | MIMAT0000682 | 0.603 | −0.73 | −39.71 | 0.41 |
| hsa-miR-200b-3p | MIMAT0000318 | 0.555 | −0.85 | −44.52 | 0.39 |
| hsa-miR-200c-3p | MIMAT0000617 | 0.847 | −0.24 | −15.33 | 0.40 |
| hsa-miR-202-3p | MIMAT0002811 | 0.927 | −0.11 | −7.34 | 0.62 |
| hsa-miR-203a-3p | MIMAT0000264 | 0.841 | −0.25 | −15.91 | 0.29 |
| hsa-miR-203a-5p | MIMAT0031890 | 0.979 | −0.03 | −2.06 | 0.97 |
| hsa-miR-204-5p | MIMAT0000265 | 0.547 | −0.87 | −45.29 | 0.40 |
| hsa-miR-205-5p | MIMAT0000266 | 0.582 | −0.78 | −41.76 | 0.61 |
| hsa-miR-208b-5p | MIMAT0026722 | 0.927 | −0.11 | −7.34 | 0.62 |
| hsa-miR-20a-5p | MIMAT0000075 | 0.651 | −0.62 | −34.93 | 0.70 |
| hsa-miR-21-5p | MIMAT0000076 | 0.651 | −0.62 | −34.93 | 0.53 |
| hsa-miR-210-5p | MIMAT0026475 | 0.979 | −0.03 | −2.06 | 0.96 |
| hsa-miR-211-3p | MIMAT0022694 | 0.927 | −0.11 | −7.34 | 0.62 |
| hsa-miR-211-5p | MIMAT0000268 | 0.946 | −0.08 | −5.39 | 0.88 |
| hsa-miR-2110 | MIMAT0010133 | 0.927 | −0.11 | −7.34 | 0.62 |
| hsa-miR-2113 | MIMAT0009206 | 0.927 | −0.11 | −7.34 | 0.62 |
| hsa-miR-2116-5p | MIMAT0011160 | 0.927 | −0.11 | −7.34 | 0.62 |
| hsa-miR-548ar-5p | MIMAT0022265 | 0.426 | −1.23 | −57.37 | 1.34E−02 |
| hsa-miR-214-3p | MIMAT0000271 | 0.620 | −0.69 | −38.01 | 0.65 |
| hsa-miR-215-5p | MIMAT0000272 | 0.732 | −0.45 | −26.80 | 0.23 |
| hsa-miR-146a-5p | MIMAT0000449 | 0.586 | −0.77 | −41.36 | 0.37 |
| hsa-miR-217 | MIMAT0000274 | 0.927 | −0.11 | −7.34 | 0.62 |
| hsa-miR-335-5p | MIMAT0000765 | 0.285 | −1.81 | −71.48 | 0.16 |
| hsa-miR-219a-1-3p | MIMAT0004567 | 0.927 | −0.11 | −7.34 | 0.62 |
| hsa-miR-219a-2-3p | MIMAT0004675 | 0.774 | −0.37 | −22.62 | 0.14 |
| hsa-miR-219a-5p | MIMAT0000276 | 0.927 | −0.11 | −7.34 | 0.62 |
| hsa-miR-22-3p | MIMAT0000077 | 0.409 | −1.29 | −59.10 | 0.23 |
| hsa-miR-221-3p | MIMAT0000278 | 0.476 | −1.07 | −52.37 | 0.48 |
| hsa-miR-221-5p | MIMAT0004568 | 0.927 | −0.11 | −7.34 | 0.62 |
| hsa-miR-222-3p | MIMAT0000279 | 0.285 | −1.81 | −71.48 | 0.36 |
| hsa-miR-223-3p | MIMAT0000280 | 0.423 | −1.24 | −57.66 | 0.60 |
| hsa-miR-224-5p | MIMAT0000281 | 0.927 | −0.11 | −7.34 | 0.62 |
| hsa-miR-2278 | MIMAT0011778 | 0.927 | −0.11 | −7.34 | 0.62 |
| hsa-miR-660-5p | MIMAT0003338 | 0.629 | −0.67 | −37.15 | 0.69 |
| hsa-miR-377-3p | MIMAT0000730 | 0.543 | −0.88 | −45.66 | 0.37 |
| hsa-miR-23c | MIMAT0018000 | 0.927 | −0.11 | −7.34 | 0.62 |
| hsa-miR-145-5p | MIMAT0000437 | 0.321 | −1.64 | −67.91 | 0.14 |
| hsa-miR-218-5p | MIMAT0000275 | 0.486 | −1.04 | −51.37 | 0.41 |
| hsa-miR-25-5p | MIMAT0004498 | 0.586 | −0.77 | −41.36 | 0.28 |
| hsa-miR-26a-5p | MIMAT0000082 | 0.366 | −1.45 | −63.40 | 0.42 |
| hsa-miR-26b-5p | MIMAT0000083 | 0.435 | −1.2 | −56.47 | 0.34 |
| hsa-miR-27a-3p | MIMAT0000084 | 0.559 | −0.84 | −44.14 | 0.23 |
| hsa-miR-27b-3p | MIMAT0000419 | 0.595 | −0.75 | −40.54 | 0.49 |
| hsa-miR-28-3p | MIMAT0004502 | 0.616 | −0.7 | −38.44 | 0.47 |
| hsa-miR-28-5p | MIMAT0000085 | 0.895 | −0.16 | −10.50 | 0.72 |
| hsa-miR-296-3p | MIMAT0004679 | 0.927 | −0.11 | −7.34 | 0.62 |
| hsa-miR-296-5p | MIMAT0000690 | 0.927 | −0.11 | −7.34 | 0.62 |
| hsa-miR-297 | MIMAT0004450 | 0.927 | −0.11 | −7.34 | 0.62 |
| hsa-miR-298 | MIMAT0004901 | 0.927 | −0.11 | −7.34 | 0.62 |
| hsa-miR-299-3p | MIMAT0000687 | 0.824 | −0.28 | −17.64 | 0.83 |
| hsa-miR-29a-3p | MIMAT0000086 | 0.559 | −0.84 | −44.14 | 0.45 |
| hsa-miR-495-3p | MIMAT0002817 | 0.476 | −1.07 | −52.37 | 0.20 |
| hsa-miR-29c-3p | MIMAT0000681 | 0.629 | −0.67 | −37.15 | 0.58 |
| hsa-miR-300 | MIMAT0004903 | 0.927 | −0.11 | −7.34 | 0.62 |
| hsa-miR-301a-3p | MIMAT0000688 | 0.732 | −0.45 | −26.80 | 0.69 |
| hsa-miR-301a-5p | MIMAT0022696 | 0.697 | −0.52 | −30.26 | 0.64 |
| hsa-miR-301b-3p | MIMAT0004958 | 0.927 | −0.11 | −7.34 | 0.62 |
| hsa-miR-301b-5p | MIMAT0032026 | 0.927 | −0.11 | −7.34 | 0.62 |
| hsa-miR-302a-3p | MIMAT0000684 | 0.927 | −0.11 | −7.34 | 0.62 |
| hsa-miR-302a-5p | MIMAT0000683 | 0.927 | −0.11 | −7.34 | 0.62 |
| hsa-miR-302b-3p | MIMAT0000715 | 0.737 | −0.44 | −26.29 | 0.55 |
| hsa-miR-302c-3p | MIMAT0000717 | 0.927 | −0.11 | −7.34 | 0.62 |
| hsa-miR-302d-3p | MIMAT0000718 | 0.933 | −0.1 | −6.70 | 0.70 |
| hsa-miR-302e | MIMAT0005931 | 0.927 | −0.11 | −7.34 | 0.62 |
| hsa-miR-302f | MIMAT0005932 | 0.927 | −0.11 | −7.34 | 0.62 |
| hsa-miR-3065-3p | MIMAT0015378 | 0.927 | −0.11 | −7.34 | 0.62 |
| hsa-miR-3065-5p | MIMAT0015066 | 0.927 | −0.11 | −7.34 | 0.62 |
| hsa-miR-3074-3p | MIMAT0015027 | 0.812 | −0.3 | −18.77 | 0.26 |
| hsa-miR-30a-3p | MIMAT0000088 | 0.486 | −1.04 | −51.37 | 0.50 |
| hsa-let-7g-5p | MIMAT0000414 | 0.285 | −1.81 | −71.48 | 0.12 |
| hsa-miR-30b-5p | MIMAT0000420 | 0.395 | −1.34 | −60.50 | 0.23 |
| hsa-miR-30c-5p | MIMAT0000244 | 0.674 | −0.57 | −32.64 | 0.60 |
| hsa-miR-127-3p | MIMAT0000446 | 0.358 | −1.48 | −64.15 | 0.28 |
| hsa-miR-4286 | MIMAT0016916 | 0.460 | −1.12 | −53.99 | 7.07E−02 |
| hsa-miR-30e-5p | MIMAT0000692 | 0.500 | −1 | −50.00 | 0.31 |
| hsa-miR-31-5p | MIMAT0000089 | 0.927 | −0.11 | −7.34 | 0.62 |
| hsa-miR-3127-5p | MIMAT0014990 | 0.927 | −0.11 | −7.34 | 0.62 |
| hsa-miR-3130-3p | MIMAT0014994 | 0.927 | −0.11 | −7.34 | 0.62 |
| hsa-miR-3131 | MIMAT0014996 | 0.927 | −0.11 | −7.34 | 0.62 |
| hsa-miR-3136-5p | MIMAT0015003 | 0.927 | −0.11 | −7.34 | 0.62 |
| hsa-miR-3140-3p | MIMAT0015008 | 0.927 | −0.11 | −7.34 | 0.62 |
| hsa-miR-3140-5p | MIMAT0019204 | 0.927 | −0.11 | −7.34 | 0.62 |
| hsa-miR-3144-5p | MIMAT0015014 | 0.927 | −0.11 | −7.34 | 0.62 |
| hsa-miR-3147 | MIMAT0015019 | 0.927 | −0.11 | −7.34 | 0.62 |
| hsa-miR-3150b-3p | MIMAT0018194 | 0.927 | −0.11 | −7.34 | 0.62 |
| hsa-miR-3151-5p | MIMAT0015024 | 0.563 | −0.83 | −43.75 | 0.13 |
| hsa-miR-3158-3p | MIMAT0015032 | 0.835 | −0.26 | −16.49 | 0.89 |
| hsa-miR-3161 | MIMAT0015035 | 0.927 | −0.11 | −7.34 | 0.62 |
| hsa-miR-3164 | MIMAT0015038 | 0.927 | −0.11 | −7.34 | 0.62 |
| hsa-miR-3168 | MIMAT0015043 | 0.920 | −0.12 | −7.98 | 0.86 |
| hsa-miR-3179 | MIMAT0015056 | 0.927 | −0.11 | −7.34 | 0.62 |
| hsa-miR-3180-3p | MIMAT0015058 | 0.927 | −0.11 | −7.34 | 0.62 |
| hsa-miR-3180-5p | MIMAT0015057 | 0.693 | −0.53 | −30.74 | 0.17 |
| hsa-miR-3185 | MIMAT0015065 | 0.927 | −0.11 | −7.34 | 0.62 |
| hsa-miR-3190-3p | MIMAT0022839 | 0.927 | −0.11 | −7.34 | 0.62 |
| hsa-miR-3192-5p | MIMAT0015076 | 0.927 | −0.11 | −7.34 | 0.62 |
| hsa-miR-3195 | MIMAT0015079 | 0.732 | −0.45 | −26.80 | 0.23 |
| hsa-miR-3196 | MIMAT0015080 | 0.927 | −0.11 | −7.34 | 0.62 |
| hsa-miR-32-5p | MIMAT0000090 | 0.500 | −1 | −50.00 | 0.38 |
| hsa-miR-3202 | MIMAT0015089 | 0.927 | −0.11 | −7.34 | 0.62 |
| hsa-miR-320a | MIMAT0000510 | 0.722 | −0.47 | −27.80 | 0.23 |
| hsa-miR-320b | MIMAT0005792 | 0.927 | −0.11 | −7.34 | 0.62 |
| hsa-miR-320c | MIMAT0005793 | 0.927 | −0.11 | −7.34 | 0.62 |
| hsa-miR-320d | MIMAT0006764 | 0.785 | −0.35 | −21.54 | 0.24 |
| hsa-miR-320e | MIMAT0015072 | 0.940 | −0.09 | −6.05 | 0.92 |
| hsa-let-7e-5p | MIMAT0000066 | 0.480 | −1.06 | −52.04 | 4.53E−02 |
| hsa-miR-323a-5p | MIMAT0004696 | 0.927 | −0.11 | −7.34 | 0.62 |
| hsa-miR-323b-3p | MIMAT0015050 | 0.901 | −0.15 | −9.87 | 0.90 |
| hsa-miR-323b-5p | MIMAT0001630 | 0.927 | −0.11 | −7.34 | 0.62 |
| hsa-miR-324-3p | MIMAT0000762 | 0.829 | −0.27 | −17.07 | 0.28 |
| hsa-miR-324-5p | MIMAT0000761 | 0.384 | −1.38 | −61.58 | 0.43 |
| hsa-miR-326 | MIMAT0000756 | 0.927 | −0.11 | −7.34 | 0.62 |
| hsa-miR-328-3p | MIMAT0000752 | 0.624 | −0.68 | −37.58 | 0.14 |
| hsa-miR-328-5p | MIMAT0026486 | 0.889 | −0.17 | −11.12 | 0.45 |
| hsa-miR-329-3p | MIMAT0001629 | 0.829 | −0.27 | −17.07 | 0.28 |
| hsa-miR-329-5p | MIMAT0026555 | 0.790 | −0.34 | −21.00 | 0.24 |
| hsa-miR-330-3p | MIMAT0000751 | 0.927 | −0.11 | −7.34 | 0.62 |
| hsa-miR-330-5p | MIMAT0004693 | 0.927 | −0.11 | −7.34 | 0.62 |
| hsa-miR-331-3p | MIMAT0000760 | 0.927 | −0.11 | −7.34 | 0.62 |
| hsa-miR-331-5p | MIMAT0004700 | 0.927 | −0.11 | −7.34 | 0.62 |
| hsa-miR-148a-3p | MIMAT0000243 | 0.470 | −1.09 | −53.02 | 0.23 |
| hsa-miR-337-3p | MIMAT0000754 | 0.607 | −0.72 | −39.29 | 0.37 |
| hsa-miR-337-5p | MIMAT0004695 | 0.607 | −0.72 | −39.29 | 0.64 |
| hsa-miR-338-5p | MIMAT0004701 | 0.758 | −0.4 | −24.21 | 0.33 |
| hsa-miR-339-3p | MIMAT0004702 | 0.927 | −0.11 | −7.34 | 0.62 |
| hsa-miR-339-5p | MIMAT0000764 | 0.927 | −0.11 | −7.34 | 0.62 |
| hsa-miR-33a-5p | MIMAT0000091 | 0.753 | −0.41 | −24.74 | 0.65 |
| hsa-miR-340-5p | MIMAT0004692 | 0.669 | −0.58 | −33.10 | 0.41 |
| hsa-miR-342-3p | MIMAT0000753 | 0.500 | −1 | −50.00 | 0.31 |
| hsa-miR-342-5p | MIMAT0004694 | 0.927 | −0.11 | −7.34 | 0.62 |
| hsa-miR-345-3p | MIMAT0022698 | 0.859 | −0.22 | −14.14 | 0.33 |
| hsa-miR-345-5p | MIMAT0000772 | 0.807 | −0.31 | −19.34 | 0.20 |
| hsa-miR-34a-5p | MIMAT0000255 | 0.595 | −0.75 | −40.54 | 0.45 |
| hsa-miR-34b-3p | MIMAT0004676 | 0.927 | −0.11 | −7.34 | 0.62 |
| hsa-miR-34c-3p | MIMAT0004677 | 0.927 | −0.11 | −7.34 | 0.62 |
| hsa-miR-34c-5p | MIMAT0000686 | 0.927 | −0.11 | −7.34 | 0.62 |
| hsa-miR-3605-3p | MIMAT0017982 | 0.927 | −0.11 | −7.34 | 0.62 |
| hsa-miR-3605-5p | MIMAT0017981 | 0.927 | −0.11 | −7.34 | 0.62 |
| hsa-miR-361-3p | MIMAT0004682 | 0.356 | −1.49 | −64.40 | 0.36 |
| hsa-miR-497-5p | MIMAT0002820 | 0.574 | −0.8 | −42.57 | 0.43 |
| hsa-miR-3613-3p | MIMAT0017991 | 0.927 | −0.11 | −7.34 | 0.62 |
| hsa-miR-3613-5p | MIMAT0017990 | 0.927 | −0.11 | −7.34 | 0.62 |
| hsa-miR-3614-3p | MIMAT0017993 | 0.927 | −0.11 | −7.34 | 0.62 |
| hsa-miR-3614-5p | MIMAT0017992 | 0.933 | −0.1 | −6.70 | 0.90 |
| hsa-miR-3615 | MIMAT0017994 | 0.927 | −0.11 | −7.34 | 0.62 |
| hsa-miR-362-3p | MIMAT0004683 | 0.563 | −0.83 | −43.75 | 0.48 |
| hsa-miR-362-5p | MIMAT0000705 | 0.895 | −0.16 | −10.50 | 0.45 |
| hsa-miR-363-3p | MIMAT0000707 | 0.448 | −1.16 | −55.25 | 0.30 |
| hsa-miR-363-5p | MIMAT0003385 | 0.927 | −0.11 | −7.34 | 0.62 |
| hsa-miR-365a-3p | MIMAT0000710 | 0.387 | −1.37 | −61.31 | 0.19 |
| hsa-miR-365b-5p | MIMAT0022833 | 0.927 | −0.11 | −7.34 | 0.62 |
| hsa-miR-367-3p | MIMAT0000719 | 0.841 | −0.25 | −15.91 | 0.29 |
| hsa-miR-664a-3p | MIMAT0005949 | 0.500 | −1 | −50.00 | 5.89E−02 |
| hsa-miR-369-5p | MIMAT0001621 | 0.927 | −0.11 | −7.34 | 0.62 |
| hsa-miR-3690 | MIMAT0018119 | 0.927 | −0.11 | −7.34 | 0.62 |
| hsa-miR-370-5p | MIMAT0026483 | 0.927 | −0.11 | −7.34 | 0.62 |
| hsa-miR-371a-5p | MIMAT0004687 | 0.927 | −0.11 | −7.34 | 0.62 |
| hsa-miR-371b-5p | MIMAT0019892 | 0.927 | −0.11 | −7.34 | 0.62 |
| hsa-miR-372-3p | MIMAT0000724 | 0.927 | −0.11 | −7.34 | 0.62 |
| hsa-mIR-373-3p | MIMAT0000726 | 0.927 | −0.11 | −7.34 | 0.62 |
| hsa-miR-374a-3p | MIMAT0004688 | 0.927 | −0.11 | −7.34 | 0.62 |
| hsa-miR-374a-5p | MIMAT0000727 | 0.274 | −1.87 | −72.64 | 0.36 |
| hsa-miR-374b-5p | MIMAT0004955 | 0.790 | −0.34 | −21.00 | 0.64 |
| hsa-miR-374c-5p | MIMAT0018443 | 0.927 | −0.11 | −7.34 | 0.62 |
| hsa-miR-375 | MIMAT0000728 | 0.927 | −0.11 | −7.34 | 0.62 |
| hsa-miR-376a-3p | MIMAT0000729 | 0.747 | −0.42 | −25.26 | 0.68 |
| hsa-miR-376b-3p | MIMAT0002172 | 0.927 | −0.11 | −7.34 | 0.62 |
| hsa-miR-376c-3p | MIMAT0000720 | 0.586 | −0.77 | −41.36 | 0.64 |
| hsa-miR-376c-5p | MIMAT0022861 | 0.927 | −0.11 | −7.34 | 0.62 |
| hsa-miR-378b | MIMAT0014999 | 0.812 | −0.3 | −18.77 | 0.26 |
| hsa-miR-378c | MIMAT0016847 | 0.927 | −0.11 | −7.34 | 0.62 |
| hsa-miR-378d | MIMAT0018926 | 0.470 | −1.09 | −53.02 | 0.30 |
| hsa-miR-378f | MIMAT0018932 | 0.563 | −0.83 | −43.75 | 0.48 |
| hsa-miR-378g | MIMAT0018937 | 0.801 | −0.32 | −19.89 | 0.65 |
| hsa-miR-378h | MIMAT0018984 | 0.812 | −0.3 | −18.77 | 0.26 |
| hsa-miR-378i | MIMAT0019074 | 0.529 | −0.92 | −47.15 | 0.36 |
| hsa-miR-379-5p | MIMAT0000733 | 0.979 | −0.03 | −2.06 | 0.96 |
| hsa-miR-380-3p | MIMAT0000735 | 0.927 | −0.11 | −7.34 | 0.62 |
| hsa-miR-381-5p | MIMAT0022862 | 0.927 | −0.11 | −7.34 | 0.62 |
| hsa-miR-382-3p | MIMAT0022697 | 0.889 | −0.17 | −11.12 | 0.45 |
| hsa-miR-382-5p | MIMAT0000737 | 0.586 | −0.77 | −41.36 | 0.55 |
| hsa-miR-383-5p | MIMAT0000738 | 0.927 | −0.11 | −7.34 | 0.62 |
| hsa-miR-384 | MIMAT0001075 | 0.927 | −0.11 | −7.34 | 0.62 |
| hsa-miR-3916 | MIMAT0018190 | 0.927 | −0.11 | −7.34 | 0.62 |
| hsa-miR-3918 | MIMAT0018192 | 0.927 | −0.11 | −7.34 | 0.62 |
| hsa-miR-3928-3p | MIMAT0018205 | 0.927 | −0.11 | −7.34 | 0.62 |
| hsa-miR-3934-5p | MIMAT0018349 | 0.927 | −0.11 | −7.34 | 0.62 |
| hsa-miR-409-3p | MIMAT0001639 | 0.712 | −0.49 | −28.80 | 0.24 |
| hsa-miR-409-5p | MIMAT0001638 | 0.927 | −0.11 | −7.34 | 0.62 |
| hsa-miR-410-3p | MIMAT0002171 | 0.927 | −0.11 | −7.34 | 0.62 |
| hsa-miR-412-3p | MIMAT0002170 | 0.927 | −0.11 | −7.34 | 0.62 |
| hsa-miR-422a | MIMAT0001339 | 0.865 | −0.21 | −13.55 | 0.35 |
| hsa-miR-423-3p | MIMAT0001340 | 0.732 | −0.45 | −26.80 | 0.64 |
| hsa-miR-423-5p | MIMAT0004748 | 0.420 | −1.25 | −57.96 | 0.33 |
| hsa-miR-130a-3p | MIMAT0000425 | 0.547 | −0.87 | −45.29 | 0.38 |
| hsa-miR-425-5p | MIMAT0003393 | 0.624 | −0.68 | −37.58 | 0.40 |
| hsa-miR-4284 | MIMAT0016915 | 0.835 | −0.26 | −16.49 | 0.25 |
| hsa-miR-190a-5p | MIMAT0000458 | 0.503 | −0.99 | −49.65 | 3.96E−02 |
| hsa-miR-429 | MIMAT0001536 | 0.444 | −1.17 | −55.56 | 0.11 |
| hsa-miR-431-5p | MIMAT0001625 | 0.927 | −0.11 | −7.34 | 0.62 |
| hsa-miR-432-5p | MIMAT0002814 | 0.790 | −0.34 | −21.00 | 0.16 |
| hsa-miR-433-3p | MIMAT0001627 | 0.927 | −0.11 | −7.34 | 0.62 |
| hsa-miR-433-5p | MIMAT0026554 | 0.927 | −0.11 | −7.34 | 0.62 |
| hsa-miR-2117 | MIMAT0011162 | 0.507 | −0.98 | −49.30 | 3.29E−02 |
| hsa-miR-4425 | MIMAT0018940 | 0.927 | −0.11 | −7.34 | 0.62 |
| hsa-miR-4431 | MIMAT0018947 | 0.927 | −0.11 | −7.34 | 0.62 |
| hsa-miR-4435 | MIMAT0018951 | 0.927 | −0.11 | −7.34 | 0.62 |
| hsa-miR-548z | MIMAT0018446 | 0.511 | −0.97 | −48.95 | 1.97E−02 |
| hsa-miR-4448 | MIMAT0018967 | 0.927 | −0.11 | −7.34 | 0.62 |
| hsa-miR-25-3p | MIMAT0000081 | 0.607 | −0.72 | −39.29 | 0.31 |
| hsa-miR-1323 | MIMAT0005795 | 0.514 | −0.96 | −48.59 | 4.92E−02 |
| hsa-miR-4458 | MIMAT0018980 | 0.927 | −0.11 | −7.34 | 0.62 |
| hsa-miR-4461 | MIMAT0018983 | 0.927 | −0.11 | −7.34 | 0.62 |
| hsa-miR-4485-3p | MIMAT0019019 | 0.927 | −0.11 | −7.34 | 0.62 |
| hsa-miR-449a | MIMAT0001541 | 0.927 | −0.11 | −7.34 | 0.62 |
| hsa-miR-449b-5p | MIMAT0003327 | 0.927 | −0.11 | −7.34 | 0.62 |
| hsa-miR-449c-5p | MIMAT0010251 | 0.927 | −0.11 | −7.34 | 0.62 |
| hsa-miR-450a-1-3p | MIMAT0022700 | 0.859 | −0.22 | −14.14 | 0.33 |
| hsa-miR-450b-3p | MIMAT0004910 | 0.927 | −0.11 | −7.34 | 0.62 |
| hsa-miR-4516 | MIMAT0019053 | 0.366 | −1.45 | −63.40 | 0.15 |
| hsa-miR-451a | MIMAT0001631 | 0.785 | −0.35 | −21.54 | 0.73 |
| hsa-miR-487a-3p | MIMAT0002178 | 0.532 | −0.91 | −46.78 | 3.77E−02 |
| hsa-miR-4521 | MIMAT0019058 | 0.927 | −0.11 | −7.34 | 0.62 |
| hsa-miR-4524a-5p | MIMAT0019062 | 0.927 | −0.11 | −7.34 | 0.62 |
| hsa-miR-4532 | MIMAT0019071 | 0.420 | −1.25 | −57.96 | 0.46 |
| hsa-miR-4536-3p | MIMAT0020959 | 0.859 | −0.22 | −14.14 | 0.33 |
| hsa-miR-454-3p | MIMAT0003885 | 0.779 | −0.36 | −22.08 | 0.24 |
| hsa-miR-455-3p | MIMAT0004784 | 0.624 | −0.68 | −37.58 | 0.25 |
| hsa-miR-455-5p | MIMAT0003150 | 0.722 | −0.47 | −27.80 | 0.74 |
| hsa-miR-4647 | MIMAT0019709 | 0.927 | −0.11 | −7.34 | 0.62 |
| hsa-miR-4707-3p | MIMAT0019808 | 0.927 | −0.11 | −7.34 | 0.62 |
| hsa-miR-4707-5p | MIMAT0019807 | 0.812 | −0.3 | −18.77 | 0.37 |
| hsa-miR-4741 | MIMAT0019871 | 0.927 | −0.11 | −7.34 | 0.62 |
| hsa-miR-4787-3p | MIMAT0019957 | 0.927 | −0.11 | −7.34 | 0.62 |
| hsa-miR-4792 | MIMAT0019964 | 0.927 | −0.11 | −7.34 | 0.62 |
| hsa-miR-483-3p | MIMAT0002173 | 0.774 | −0.37 | −22.62 | 0.23 |
| hsa-miR-483-5p | MIMAT0004761 | 0.722 | −0.47 | −27.80 | 0.23 |
| hsa-miR-484 | MIMAT0002174 | 0.747 | −0.42 | −25.26 | 0.23 |
| hsa-miR-485-5p | MIMAT0002175 | 0.927 | −0.11 | −7.34 | 0.62 |
| hsa-miR-486-3p | MIMAT0004762 | 0.753 | −0.41 | −24.74 | 0.78 |
| hsa-miR-4443 | MIMAT0018961 | 0.540 | −0.89 | −46.04 | 5.50E−02 |
| hsa-miR-487b-3p | MIMAT0003180 | 0.655 | −0.61 | −34.48 | 0.15 |
| hsa-miR-487b-5p | MIMAT0026614 | 0.927 | −0.11 | −7.34 | 0.62 |
| hsa-miR-489-3p | MIMAT0002805 | 0.927 | −0.11 | −7.34 | 0.62 |
| hsa-miR-490-3p | MIMAT0002806 | 0.927 | −0.11 | −7.34 | 0.62 |
| hsa-miR-490-5p | MIMAT0004764 | 0.927 | −0.11 | −7.34 | 0.62 |
| hsa-miR-491-3p | MIMAT0004765 | 0.835 | −0.26 | −16.49 | 0.34 |
| hsa-miR-491-5p | MIMAT0002807 | 0.841 | −0.25 | −15.91 | 0.84 |
| hsa-miR-492 | MIMAT0002812 | 0.927 | −0.11 | −7.34 | 0.62 |
| hsa-miR-493-3p | MIMAT0003161 | 0.927 | −0.11 | −7.34 | 0.62 |
| hsa-miR-494-3p | MIMAT0002816 | 0.683 | −0.55 | −31.70 | 0.24 |
| hsa-miR-494-5p | MIMAT0026607 | 0.927 | −0.11 | −7.34 | 0.62 |
| hsa-miR-543 | MIMAT0004954 | 0.824 | −0.28 | −17.64 | 0.43 |
| hsa-miR-496 | MIMAT0002818 | 0.865 | −0.21 | −13.55 | 0.81 |
| hsa-let-7f-5p | MIMAT0000067 | 0.543 | −0.88 | −45.66 | 9.44E−02 |
| hsa-miR-498 | MIMAT0002824 | 0.927 | −0.11 | −7.34 | 0.62 |
| hsa-miR-499a-3p | MIMAT0004772 | 0.927 | −0.11 | −7.34 | 0.62 |
| hsa-miR-499a-5p | MIMAT0002870 | 0.940 | −0.09 | −6.05 | 0.97 |
| hsa-miR-499b-3p | MIMAT0019898 | 0.927 | −0.11 | −7.34 | 0.62 |
| hsa-miR-499b-5p | MIMAT0019897 | 0.927 | −0.11 | −7.34 | 0.62 |
| hsa-miR-5001-3p | MIMAT0021022 | 0.927 | −0.11 | −7.34 | 0.62 |
| hsa-miR-5001-5p | MIMAT0021021 | 0.927 | −0.11 | −7.34 | 0.62 |
| hsa-miR-500a-5p | MIMAT0004773 | 0.835 | −0.26 | −16.49 | 0.24 |
| hsa-miR-501-3p | MIMAT0004774 | 0.927 | −0.11 | −7.34 | 0.62 |
| hsa-miR-5010-3p | MIMAT0021044 | 0.889 | −0.17 | −11.12 | 0.45 |
| hsa-miR-5010-5p | MIMAT0021043 | 0.927 | −0.11 | −7.34 | 0.62 |
| hsa-miR-502-3p | MIMAT0004775 | 0.927 | −0.11 | −7.34 | 0.62 |
| hsa-miR-502-5p | MIMAT0002873 | 0.927 | −0.11 | −7.34 | 0.62 |
| hsa-miR-503-3p | MIMAT0022925 | 0.927 | −0.11 | −7.34 | 0.62 |
| hsa-miR-503-5p | MIMAT0002874 | 0.737 | −0.44 | −26.29 | 0.66 |
| hsa-miR-504-3p | MIMAT0026612 | 0.927 | −0.11 | −7.34 | 0.62 |
| hsa-miR-504-5p | MIMAT0002875 | 0.927 | −0.11 | −7.34 | 0.62 |
| hsa-miR-148b-3p | MIMAT0000759 | 0.574 | −0.8 | −42.57 | 0.35 |
| hsa-miR-506-5p | MIMAT0022701 | 0.927 | −0.11 | −7.34 | 0.62 |
| hsa-miR-507 | MIMAT0002879 | 0.927 | −0.11 | −7.34 | 0.62 |
| hsa-miR-508-3p | MIMAT0002880 | 0.927 | −0.11 | −7.34 | 0.62 |
| hsa-miR-508-5p | MIMAT0004778 | 0.927 | −0.11 | −7.34 | 0.62 |
| hsa-miR-509-3-5p | MIMAT0004975 | 0.927 | −0.11 | −7.34 | 0.62 |
| hsa-miR-509-3p | MIMAT0002881 | 0.379 | −1.4 | −62.11 | 0.34 |
| hsa-miR-509-5p | MIMAT0004779 | 0.664 | −0.59 | −33.57 | 0.12 |
| hsa-miR-510-3p | MIMAT0026613 | 0.927 | −0.11 | −7.34 | 0.62 |
| hsa-miR-510-5p | MIMAT0002882 | 0.927 | −0.11 | −7.34 | 0.62 |
| hsa-miR-511-5p | MIMAT0002808 | 0.758 | −0.4 | −24.21 | 0.12 |
| hsa-miR-512-3p | MIMAT0002823 | 0.927 | −0.11 | −7.34 | 0.62 |
| hsa-miR-512-5p | MIMAT0002822 | 0.927 | −0.11 | −7.34 | 0.62 |
| hsa-miR-513a-5p | MIMAT0002877 | 0.966 | −0.05 | −3.41 | 0.90 |
| hsa-miR-513b-5p | MIMAT0005788 | 0.655 | −0.61 | −34.48 | 0.65 |
| hsa-miR-513c-3p | MIMAT0022728 | 0.927 | −0.11 | −7.34 | 0.62 |
| hsa-miR-513c-5p | MIMAT0005789 | 0.525 | −0.93 | −47.51 | 0.20 |
| hsa-miR-514a-3p | MIMAT0002883 | 0.470 | −1.09 | −53.02 | 0.16 |
| hsa-miR-514a-5p | MIMAT0022702 | 0.688 | −0.54 | −31.22 | 0.70 |
| hsa-miR-514b-3p | MIMAT0015088 | 0.927 | −0.11 | −7.34 | 0.62 |
| hsa-miR-514b-5p | MIMAT0015087 | 0.674 | −0.57 | −32.64 | 0.66 |
| hsa-miR-515-3p | MIMAT0002827 | 0.927 | −0.11 | −7.34 | 0.62 |
| hsa-miR-515-5p | MIMAT0002826 | 0.927 | −0.11 | −7.34 | 0.62 |
| hsa-miR-516a-3p | MIMAT0006778 | 0.895 | −0.16 | −10.50 | 0.45 |
| hsa-miR-516a-5p | MIMAT0004770 | 0.927 | −0.11 | −7.34 | 0.62 |
| hsa-miR-S16b-5p | MIMAT0002859 | 0.812 | −0.3 | −18.77 | 0.26 |
| hsa-miR-517a-3p | MIMAT0002852 | 0.927 | −0.11 | −7.34 | 0.62 |
| hsa-miR-517b-3p | MIMAT0002857 | 0.927 | −0.11 | −7.34 | 0.62 |
| hsa-miR-517c-3p | MIMAT0002866 | 0.847 | −0.24 | −15.33 | 0.40 |
| hsa-miR-518b | MIMAT0002844 | 0.651 | −0.62 | −34.93 | 0.69 |
| hsa-miR-518c-3p | MIMAT0002848 | 0.927 | −0.11 | −7.34 | 0.62 |
| hsa-miR-518d-3p | MIMAT0002864 | 0.927 | −0.11 | −7.34 | 0.62 |
| hsa-miR-518e-3p | MIMAT0002861 | 0.927 | −0.11 | −7.34 | 0.62 |
| hsa-miR-5196-3p | MIMAT0021129 | 0.702 | −0.51 | −29.78 | 0.16 |
| hsa-miR-5196-5p | MIMAT0021128 | 0.768 | −0.38 | −23.16 | 0.27 |
| hsa-miR-519b-3p | MIMAT0002837 | 0.927 | −0.11 | −7.34 | 0.62 |
| hsa-miR-519b-5p | MIMAT0005454 | 0.927 | −0.11 | −7.34 | 0.62 |
| hsa-miR-519c-3p | MIMAT0002832 | 0.712 | −0.49 | −28.80 | 0.18 |
| hsa-miR-519d-3p | MIMAT0002853 | 0.865 | −0.21 | −13.55 | 0.67 |
| hsa-miR-519e-3p | MIMAT0002829 | 0.927 | −0.11 | −7.34 | 0.62 |
| hsa-miR-520a-3p | MIMAT0002834 | 0.927 | −0.11 | −7.34 | 0.62 |
| hsa-miR-520a-5p | MIMAT0002833 | 0.927 | −0.11 | −7.34 | 0.62 |
| hsa-miR-520b | MIMAT0002843 | 0.927 | −0.11 | −7.34 | 0.62 |
| hsa-miR-520c-3p | MIMAT0002846 | 0.927 | −0.11 | −7.34 | 0.62 |
| hsa-miR-520d-3p | MIMAT0002856 | 0.927 | −0.11 | −7.34 | 0.62 |
| hsa-miR-520d-5p | MIMAT0002855 | 0.927 | −0.11 | −7.34 | 0.62 |
| hsa-miR-520e | MIMAT0002825 | 0.927 | −0.11 | −7.34 | 0.62 |
| hsa-miR-520g-3p | MIMAT0002858 | 0.785 | −0.35 | −21.54 | 0.32 |
| hsa-miR-520h | MIMAT0002867 | 0.774 | −0.37 | −22.62 | 0.21 |
| hsa-miR-522-3p | MIMAT0002868 | 0.927 | −0.11 | −7.34 | 0.62 |
| hsa-miR-523-3p | MIMAT0002840 | 0.927 | −0.11 | −7.34 | 0.62 |
| hsa-miR-524-3p | MIMAT0002850 | 0.927 | −0.11 | −7.34 | 0.62 |
| hsa-miR-525-3p | MIMAT0002839 | 0.678 | −0.56 | −32.17 | 0.24 |
| hsa-miR-526a | MIMAT0002845 | 0.463 | −1.11 | −53.67 | 0.30 |
| hsa-miR-526b-5p | MIMAT0002835 | 0.927 | −0.11 | −7.34 | 0.62 |
| hsa-miR-548d-5p | MIMAT0004812 | 0.551 | −0.86 | −44.90 | 5.00E−02 |
| hsa-miR-539-3p | MIMAT0022705 | 0.927 | −0.11 | −7.34 | 0.62 |
| hsa-miR-539-5p | MIMAT0003163 | 0.927 | −0.11 | −7.34 | 0.62 |
| hsa-miR-541-3p | MIMAT0004920 | 0.927 | −0.11 | −7.34 | 0.62 |
| hsa-miR-542-3p | MIMAT0003389 | 0.497 | −1.01 | −50.35 | 0.11 |
| hsa-miR-542-5p | MIMAT0003340 | 0.678 | −0.56 | −32.17 | 0.24 |
| hsa-miR-30a-5p | MIMAT0000087 | 0.555 | −0.85 | −44.52 | 0.29 |
| hsa-miR-544a | MIMAT0003164 | 0.927 | −0.11 | −7.34 | 0.62 |
| hsa-miR-545-3p | MIMAT0003165 | 0.807 | −0.31 | −19.34 | 0.79 |
| hsa-miR-548a-3p | MIMAT0003251 | 0.927 | −0.11 | −7.34 | 0.62 |
| hsa-miR-548a-5p | MIMAT0004803 | 0.927 | −0.11 | −7.34 | 0.62 |
| hsa-miR-30d-5p | MIMAT0000245 | 0.611 | −0.71 | −38.87 | 0.35 |
| hsa-miR-548ad-3p | MIMAT0018946 | 0.927 | −0.11 | −7.34 | 0.62 |
| hsa-miR-196a-5p | MIMAT0000226 | 0.578 | −0.79 | −42.17 | 3.36E−02 |
| hsa-miR-548ai | MIMAT0018989 | 0.895 | −0.16 | −10.50 | 0.45 |
| hsa-miR-548ak | MIMAT0019013 | 0.927 | −0.11 | −7.34 | 0.62 |
| hsa-miR-548al | MIMAT0019024 | 0.927 | −0.11 | −7.34 | 0.62 |
| hsa-miR-155-5p | MIMAT0000646 | 0.582 | −0.78 | −41.76 | 9.93E−02 |
| hsa-miR-136-5p | MIMAT0000448 | 0.586 | −0.77 | −41.36 | 3.36E−02 |
| hsa-miR-548b-3p | MIMAT0003254 | 0.927 | −0.11 | −7.34 | 0.62 |
| hsa-miR-548c-5p | MIMAT0004806 | 0.927 | −0.11 | −7.34 | 0.62 |
| hsa-miR-548d-3p | MIMAT0003323 | 0.927 | −0.11 | −7.34 | 0.62 |
| hsa-miR-4421 | MIMAT0018934 | 0.586 | −0.77 | −41.36 | 9.25E−02 |
| hsa-miR-548e-3p | MIMAT0005874 | 0.927 | −0.11 | −7.34 | 0.62 |
| hsa-miR-548g-3p | MIMAT0005912 | 0.841 | −0.25 | −15.91 | 0.29 |
| hsa-miR-548h-5p | MIMAT0005928 | 0.927 | −0.11 | −7.34 | 0.62 |
| hsa-miR-548i | MIMAT0005935 | 0.927 | −0.11 | −7.34 | 0.62 |
| hsa-miR-S48j-5p | MIMAT0005875 | 0.927 | −0.11 | −7.34 | 0.62 |
| hsa-miR-548k | MIMAT0005882 | 0.801 | −0.32 | −19.89 | 0.25 |
| hsa-miR-548l | MIMAT0005889 | 0.927 | −0.11 | −7.34 | 0.62 |
| hsa-miR-548m | MIMAT0005917 | 0.688 | −0.54 | −31.22 | 0.53 |
| hsa-miR-548n | MIMAT0005916 | 0.595 | −0.75 | −40.54 | 0.20 |
| hsa-miR-548o-3p | MIMAT0005919 | 0.927 | −0.11 | −7.34 | 0.62 |
| hsa-miR-548q | MIMAT0011163 | 0.927 | −0.11 | −7.34 | 0.62 |
| hsa-miR-532-3p | MIMAT0004780 | 0.611 | −0.71 | −38.87 | 9.48E−02 |
| hsa-miR-548y | MIMAT0018354 | 0.933 | −0.1 | −6.70 | 0.75 |
| hsa-miR-99b-5p | MIMAT0000689 | 0.633 | −0.66 | −36.71 | 8.79E−02 |
| hsa-miR-549a | MIMAT0003333 | 0.753 | −0.41 | −24.74 | 0.20 |
| hsa-miR-550a-5p | MIMAT0004800 | 0.927 | −0.11 | −7.34 | 0.62 |
| hsa-miR-551a | MIMAT0003214 | 0.927 | −0.11 | −7.34 | 0.62 |
| hsa-miR-551b-3p | MIMAT0003233 | 0.702 | −0.51 | −29.78 | 0.58 |
| hsa-miR-552-3p | MIMAT0003215 | 0.927 | −0.11 | −7.34 | 0.62 |
| hsa-miR-553 | MIMAT0003216 | 0.927 | −0.11 | −7.34 | 0.62 |
| hsa-miR-554 | MIMAT0003217 | 0.927 | −0.11 | −7.34 | 0.62 |
| hsa-miR-555 | MIMAT0003219 | 0.927 | −0.11 | −7.34 | 0.62 |
| hsa-miR-556-3p | MIMAT0004793 | 0.717 | −0.48 | −28.30 | 0.24 |
| hsa-miR-556-5p | MIMAT0003220 | 0.807 | −0.31 | −19.34 | 0.21 |
| hsa-miR-561-3p | MIMAT0003225 | 0.927 | −0.11 | −7.34 | 0.62 |
| hsa-miR-562 | MIMAT0003226 | 0.927 | −0.11 | −7.34 | 0.62 |
| hsa-miR-563 | MIMAT0003227 | 0.901 | −0.15 | −9.87 | 0.52 |
| hsa-miR-564 | MIMAT0003228 | 0.927 | −0.11 | −7.34 | 0.62 |
| hsa-miR-566 | MIMAT0003230 | 0.927 | −0.11 | −7.34 | 0.62 |
| hsa-miR-567 | MIMAT0003231 | 0.927 | −0.11 | −7.34 | 0.62 |
| hsa-miR-568 | MIMAT0003232 | 0.927 | −0.11 | −7.34 | 0.62 |
| hsa-miR-570-3p | MIMAT0003235 | 0.841 | −0.25 | −15.91 | 0.29 |
| hsa-miR-571 | MIMAT0003236 | 0.927 | −0.11 | −7.34 | 0.62 |
| hsa-miR-573 | MIMAT0003238 | 0.927 | −0.11 | −7.34 | 0.62 |
| hsa-miR-23a-3p | MIMAT0000078 | 0.633 | −0.66 | −36.71 | 0.41 |
| hsa-miR-574-5p | MIMAT0004795 | 0.376 | −1.41 | −62.37 | 0.36 |
| hsa-miR-575 | MIMAT0003240 | 0.796 | −0.33 | −20.45 | 0.84 |
| hsa-miR-576-3p | MIMAT0004796 | 0.859 | −0.22 | −14.14 | 0.33 |
| hsa-miR-577 | MIMAT0003242 | 0.927 | −0.11 | −7.34 | 0.62 |
| hsa-miR-578 | MIMAT0003243 | 0.737 | −0.44 | −26.29 | 0.23 |
| hsa-miR-579-3p | MIMAT0003244 | 0.927 | −0.11 | −7.34 | 0.62 |
| hsa-miR-579-5p | MIMAT0026616 | 0.889 | −0.17 | −11.12 | 0.45 |
| hsa-miR-580-3p | MIMAT0003245 | 0.927 | −0.11 | −7.34 | 0.62 |
| hsa-miR-582-3p | MIMAT0004797 | 0.927 | −0.11 | −7.34 | 0.62 |
| hsa-miR-582-5p | MIMAT0003247 | 0.812 | −0.3 | −18.77 | 0.35 |
| hsa-miR-584-3p | MIMAT0022708 | 0.688 | −0.54 | −31.22 | 0.20 |
| hsa-miR-585-3p | MIMAT0003250 | 0.712 | −0.49 | −28.80 | 0.60 |
| hsa-miR-655-3p | MIMAT0003331 | 0.646 | −0.63 | −35.38 | 8.61E−02 |
| hsa-miR-589-5p | MIMAT0004799 | 0.927 | −0.11 | −7.34 | 0.62 |
| hsa-miR-590-3p | MIMAT0004801 | 0.927 | −0.11 | −7.34 | 0.62 |
| hsa-miR-590-5p | MIMAT0003258 | 0.323 | −1.63 | −67.69 | 0.35 |
| hsa-miR-591 | MIMAT0003259 | 0.927 | −0.11 | −7.34 | 0.62 |
| hsa-miR-592 | MIMAT0003260 | 0.790 | −0.34 | −21.00 | 0.34 |
| hsa-miR-593-3p | MIMAT0004802 | 0.620 | −0.69 | −38.01 | 0.22 |
| hsa-miR-595 | MIMAT0003263 | 0.927 | −0.11 | −7.34 | 0.62 |
| hsa-miR-596 | MIMAT0003264 | 0.927 | −0.11 | −7.34 | 0.62 |
| hsa-miR-597-5p | MIMAT0003265 | 0.697 | −0.52 | −30.26 | 0.52 |
| hsa-miR-599 | MIMAT0003267 | 0.927 | −0.11 | −7.34 | 0.62 |
| hsa-miR-600 | MIMAT0003268 | 0.927 | −0.11 | −7.34 | 0.62 |
| hsa-miR-601 | MIMAT0003269 | 0.532 | −0.91 | −46.78 | 0.36 |
| hsa-miR-604 | MIMAT0003272 | 0.927 | −0.11 | −7.34 | 0.62 |
| hsa-miR-605-5p | MIMAT0003273 | 0.927 | −0.11 | −7.34 | 0.62 |
| hsa-miR-606 | MIMAT0003274 | 0.927 | −0.11 | −7.34 | 0.62 |
| hsa-miR-607 | MIMAT0003275 | 0.768 | −0.38 | −23.16 | 0.68 |
| hsa-miR-610 | MIMAT0003278 | 0.927 | −0.11 | −7.34 | 0.62 |
| hsa-miR-613 | MIMAT0003281 | 0.927 | −0.11 | −7.34 | 0.62 |
| hsa-miR-614 | MIMAT0003282 | 0.927 | −0.11 | −7.34 | 0.62 |
| hsa-miR-615-3p | MIMAT0003283 | 0.768 | −0.38 | −23.16 | 0.33 |
| hsa-miR-615-5p | MIMAT0004804 | 0.927 | −0.11 | −7.34 | 0.62 |
| hsa-miR-616-3p | MIMAT0004805 | 0.807 | −0.31 | −19.34 | 0.56 |
| hsa-miR-617 | MIMAT0003286 | 0.927 | −0.11 | −7.34 | 0.62 |
| hsa-miR-619-3p | MIMAT0003288 | 0.927 | −0.11 | −7.34 | 0.62 |
| hsa-miR-620 | MIMAT0003289 | 0.927 | −0.11 | −7.34 | 0.62 |
| hsa-miR-624-3p | MIMAT0004807 | 0.927 | −0.11 | −7.34 | 0.62 |
| hsa-miR-626 | MIMAT0003295 | 0.927 | −0.11 | −7.34 | 0.62 |
| hsa-miR-627-5p | MIMAT0003296 | 0.790 | −0.34 | −21.00 | 0.19 |
| hsa-miR-628-3p | MIMAT0003297 | 0.927 | −0.11 | −7.34 | 0.62 |
| hsa-miR-628-5p | MIMAT0004809 | 0.927 | −0.11 | −7.34 | 0.62 |
| hsa-miR-629-5p | MIMAT0004810 | 0.927 | −0.11 | −7.34 | 0.62 |
| hsa-miR-630 | MIMAT0003299 | 0.476 | −1.07 | −52.37 | 0.62 |
| hsa-miR-631 | MIMAT0003300 | 0.847 | −0.24 | −15.33 | 0.40 |
| hsa-miR-637 | MIMAT0003307 | 0.927 | −0.11 | −7.34 | 0.62 |
| hsa-miR-638 | MIMAT0003308 | 0.927 | −0.11 | −7.34 | 0.62 |
| hsa-miR-639 | MIMAT0003309 | 0.927 | −0.11 | −7.34 | 0.62 |
| hsa-miR-640 | MIMAT0003310 | 0.927 | −0.11 | −7.34 | 0.62 |
| hsa-miR-641 | MIMAT0003311 | 0.927 | −0.11 | −7.34 | 0.62 |
| hsa-miR-642a-3p | MIMAT0020924 | 0.927 | −0.11 | −7.34 | 0.62 |
| hsa-miR-642a-5p | MIMAT0003312 | 0.753 | −0.41 | −24.74 | 0.22 |
| hsa-miR-643 | MIMAT0003313 | 0.927 | −0.11 | −7.34 | 0.62 |
| hsa-miR-644a | MIMAT0003314 | 0.927 | −0.11 | −7.34 | 0.62 |
| hsa-miR-649 | MIMAT0003319 | 0.927 | −0.11 | −7.34 | 0.62 |
| hsa-miR-650 | MIMAT0003320 | 0.927 | −0.11 | −7.34 | 0.62 |
| hsa-miR-6503-3p | MIMAT0025463 | 0.801 | −0.32 | −19.89 | 0.25 |
| hsa-miR-6503-5p | MIMAT0025462 | 0.927 | −0.11 | −7.34 | 0.62 |
| hsa-miR-651-3p | MIMAT0026624 | 0.927 | −0.11 | −7.34 | 0.62 |
| hsa-miR-651-5p | MIMAT0003321 | 0.727 | −0.46 | −27.30 | 0.37 |
| hsa-miR-6511a-3p | MIMAT0025479 | 0.927 | −0.11 | −7.34 | 0.62 |
| hsa-miR-6511a-5p | MIMAT0025478 | 0.927 | −0.11 | −7.34 | 0.62 |
| hsa-miR-652-3p | MIMAT0003322 | 0.927 | −0.11 | −7.34 | 0.62 |
| hsa-miR-652-5p | MIMAT0022709 | 0.927 | −0.11 | −7.34 | 0.62 |
| hsa-miR-654-3p | MIMAT0004814 | 0.785 | −0.35 | −21.54 | 0.24 |
| hsa-miR-654-5p | MIMAT0003330 | 0.841 | −0.25 | −15.91 | 0.38 |
| hsa-miR-587 | MIMAT0003253 | 0.660 | −0.6 | −34.02 | 9.10E−02 |
| hsa-miR-660-3p | MIMAT0022711 | 0.927 | −0.11 | −7.34 | 0.62 |
| hsa-miR-452-5p | MIMAT0001635 | 0.669 | −0.58 | −33.10 | 8.73E−02 |
| hsa-miR-661 | MIMAT0003324 | 0.927 | −0.11 | −7.34 | 0.62 |
| hsa-miR-663a | MIMAT0003326 | 0.841 | −0.25 | −15.91 | 0.88 |
| hsa-miR-548v | MIMAT0015020 | 0.693 | −0.53 | −30.74 | 9.24E−02 |
| hsa-miR-664b-3p | MIMAT0022272 | 0.927 | −0.11 | −7.34 | 0.62 |
| hsa-miR-664b-5p | MIMAT0022271 | 0.927 | −0.11 | −7.34 | 0.62 |
| hsa-miR-665 | MIMAT0004952 | 0.927 | −0.11 | −7.34 | 0.62 |
| hsa-miR-671-3p | MIMAT0004819 | 0.927 | −0.11 | −7.34 | 0.62 |
| hsa-miR-671-5p | MIMAT0003880 | 0.927 | −0.11 | −7.34 | 0.62 |
| hsa-miR-6720-3p | MIMAT0025851 | 0.927 | −0.11 | −7.34 | 0.62 |
| hsa-miR-6721-5p | MIMAT0025852 | 0.986 | −0.02 | −1.38 | 0.95 |
| hsa-miR-6724-5p | MIMAT0025856 | 0.637 | −0.65 | −36.27 | 0.10 |
| hsa-miR-675-5p | MIMAT0004284 | 0.901 | −0.15 | −9.87 | 0.52 |
| hsa-miR-7-5p | MIMAT0000252 | 0.473 | −1.08 | −52.70 | 0.14 |
| hsa-miR-708-5p | MIMAT0004926 | 0.927 | −0.11 | −7.34 | 0.62 |
| hsa-miR-744-5p | MIMAT0004945 | 0.927 | −0.11 | −7.34 | 0.62 |
| hsa-miR-758-5p | MIMAT0022929 | 0.927 | −0.11 | −7.34 | 0.62 |
| hsa-miR-760 | MIMAT0004957 | 0.927 | −0.11 | −7.34 | 0.62 |
| hsa-miR-761 | MIMAT0010364 | 0.927 | −0.11 | −7.34 | 0.62 |
| hsa-miR-764 | MIMAT0010367 | 0.920 | −0.12 | −7.98 | 0.91 |
| hsa-miR-766-3p | MIMAT0003888 | 0.927 | −0.11 | −7.34 | 0.62 |
| hsa-miR-766-5p | MIMAT0022714 | 0.927 | −0.11 | −7.34 | 0.62 |
| hsa-miR-767-3p | MIMAT0003883 | 0.927 | −0.11 | −7.34 | 0.62 |
| hsa-miR-769-3p | MIMAT0003887 | 0.927 | −0.11 | −7.34 | 0.62 |
| hsa-miR-769-5p | MIMAT0003886 | 0.540 | −0.89 | −46.04 | 0.43 |
| hsa-miR-770-5p | MIMAT0003948 | 0.927 | −0.11 | −7.34 | 0.62 |
| hsa-miR-802 | MIMAT0004185 | 0.927 | −0.11 | −7.34 | 0.62 |
| hsa-miR-874-3p | MIMAT0004911 | 0.889 | −0.17 | −11.12 | 0.45 |
| hsa-miR-874-5p | MIMAT0026718 | 0.927 | −0.11 | −7.34 | 0.62 |
| hsa-miR-875-3p | MIMAT0004923 | 0.927 | −0.11 | −7.34 | 0.62 |
| hsa-miR-876-3p | MIMAT0004925 | 0.927 | −0.11 | −7.34 | 0.62 |
| hsa-miR-876-5p | MIMAT0004924 | 0.927 | −0.11 | −7.34 | 0.62 |
| hsa-miR-877-5p | MIMAT0004949 | 0.737 | −0.44 | −26.29 | 0.55 |
| hsa-miR-885-3p | MIMAT0004948 | 0.927 | −0.11 | −7.34 | 0.62 |
| hsa-miR-885-5p | MIMAT0004947 | 0.927 | −0.11 | −7.34 | 0.62 |
| hsa-miR-887-3p | MIMAT0004951 | 0.563 | −0.83 | −43.75 | 0.10 |
| hsa-miR-887-5p | MIMAT0026720 | 0.927 | −0.11 | −7.34 | 0.62 |
| hsa-miR-888-5p | MIMAT0004916 | 0.511 | −0.97 | −48.95 | 0.21 |
| hsa-miR-889-3p | MIMAT0004921 | 0.927 | −0.11 | −7.34 | 0.62 |
| hsa-miR-890 | MIMAT0004912 | 0.927 | −0.11 | −7.34 | 0.62 |
| hsa-miR-891a-5p | MIMAT0004902 | 0.859 | −0.22 | −14.14 | 0.33 |
| hsa-miR-891b | MIMAT0004913 | 0.927 | −0.11 | −7.34 | 0.62 |
| hsa-miR-892a | MIMAT0004907 | 0.914 | −0.13 | −8.62 | 0.76 |
| hsa-miR-892b | MIMAT0004918 | 0.927 | −0.11 | −7.34 | 0.62 |
| hsa-miR-9-5p | MIMAT0000441 | 0.927 | −0.11 | −7.34 | 0.62 |
| hsa-miR-922 | MIMAT0004972 | 0.859 | −0.22 | −14.14 | 0.33 |
| hsa-miR-924 | MIMAT0004974 | 0.927 | −0.11 | −7.34 | 0.62 |
| hsa-miR-92a-1-5p | MIMAT0004507 | 0.927 | −0.11 | −7.34 | 0.62 |
| hsa-miR-92a-3p | MIMAT0000092 | 0.785 | −0.35 | −21.54 | 0.24 |
| hsa-miR-92b-3p | MIMAT0003218 | 0.927 | −0.11 | −7.34 | 0.62 |
| hsa-miR-93-5p | MIMAT0000093 | 0.451 | −1.15 | −54.94 | 0.44 |
| hsa-miR-933 | MIMAT0004976 | 0.818 | −0.29 | −18.21 | 0.33 |
| hsa-miR-934 | MIMAT0004977 | 0.927 | −0.11 | −7.34 | 0.62 |
| hsa-miR-935 | MIMAT0004978 | 0.927 | −0.11 | −7.34 | 0.62 |
| hsa-miR-936 | MIMAT0004979 | 0.865 | −0.21 | −13.55 | 0.36 |
| hsa-miR-937-3p | MIMAT0004980 | 0.927 | −0.11 | −7.34 | 0.62 |
| hsa-miR-940 | MIMAT0004983 | 0.927 | −0.11 | −7.34 | 0.62 |
| hsa-miR-941 | MIMAT0004984 | 0.927 | −0.11 | −7.34 | 0.62 |
| hsa-miR-942-3p | MIMAT0026734 | 0.927 | −0.11 | −7.34 | 0.62 |
| hsa-miR-942-5p | MIMAT0004985 | 0.927 | −0.11 | −7.34 | 0.62 |
| hsa-miR-944 | MIMAT0004987 | 0.859 | −0.22 | −14.14 | 0.33 |
| hsa-miR-95-3p | MIMAT0000094 | 0.293 | −1.77 | −70.68 | 0.31 |
| hsa-miR-96-5p | MIMAT0000095 | 0.785 | −0.35 | −21.54 | 0.37 |
| hsa-miR-98-3p | MIMAT0022842 | 0.927 | −0.11 | −7.34 | 0.62 |

TABLE 4
miRNAs Significantly Depleted using GNA Resin Side Port Flow.

| | | GNA Resin - Side Port Flow | | | |
|---|---|---|---|---|---|
| | | Fold | Log2 | Percent | |
| miRNA Name | Accession # | Change | FC | Change | p-value |
| hsa-let-7a-5p | MIMAT0000062 | 0.025 | −5.32 | −97.50 | 2.68E−02 |
| hsa-miR-29b-3p | MIMAT0000100 | 0.047 | −4.42 | −95.33 | 5.62E−02 |
| hsa-miR-99a-5p | MIMAT0000097 | 0.075 | −3.74 | −92.52 | 2.86E−02 |
| hsa-miR-15b-5p | MIMAT0000417 | 0.092 | −3.45 | −90.85 | 7.16E−02 |
| hsa-miR-548aa | MIMAT0018447 | 0.098 | −3.35 | −90.19 | 3.80E−02 |
| hsa-miR-100-5p | MIMAT0000098 | 0.136 | −2.88 | −86.42 | 8.40E−02 |
| hsa-miR-548ah-5p | MIMAT0018972 | 0.245 | −2.03 | −75.51 | 4.44E−03 |
| hsa-miR-98-5p | MIMAT0000096 | 0.255 | −1.97 | −74.47 | 5.36E−02 |
| hsa-miR-1268a | MIMAT0005922 | 0.279 | −1.84 | −72.07 | 7.22E−02 |
| hsa-miR-1290 | MIMAT0005880 | 0.301 | −1.73 | −69.85 | 5.08E−02 |
| hsa-miR-15a-5p | MIMAT0000068 | 0.321 | −1.64 | −67.91 | 9.03E−02 |
| hsa-miR-323a-3p | MIMAT0000755 | 0.344 | −1.54 | −65.61 | 5.50E−03 |
| hsa-miR-369-3p | MIMAT0000721 | 0.379 | −1.4 | −62.11 | 3.90E−02 |
| hsa-miR-193a-5p | MIMAT0004614 | 0.390 | −1.36 | −61.04 | 9.22E−02 |
| hsa-miR-126-3p | MIMAT0000445 | 0.392 | −1.35 | −60.77 | 7.95E−02 |
| hsa-miR-1972 | MIMAT0009447 | 0.403 | −1.31 | −59.67 | 2.95E−03 |
| hsa-miR-4455 | MIMAT0018977 | 0.426 | −1.23 | −57.37 | 1.84E−02 |
| hsa-miR-548ar-5p | MIMAT0022265 | 0.426 | −1.23 | −57.37 | 1.34E−02 |
| hsa-miR-4286 | MIMAT0016916 | 0.460 | −1.12 | −53.99 | 7.07E−02 |
| hsa-let-7e-5p | MIMAT0000066 | 0.480 | −1.06 | −52.04 | 4.53E−02 |
| hsa-miR-664a-3p | MIMAT0005949 | 0.500 | −1 | −50.00 | 5.89E−02 |
| hsa-miR-190a-5p | MIMAT0000458 | 0.503 | −0.99 | −49.65 | 3.96E−02 |
| hsa-miR-2117 | MIMAT0011162 | 0.507 | −0.98 | −49.30 | 3.29E−02 |
| hsa-miR-548z | MIMAT0018446 | 0.511 | −0.97 | −48.95 | 1.97E−02 |
| hsa-miR-1323 | MIMAT0005795 | 0.514 | −0.96 | −48.59 | 4.92E−02 |
| hsa-miR-487a-3p | MIMAT0002178 | 0.532 | −0.91 | −46.78 | 3.77E−02 |
| hsa-miR-4443 | MIMAT0018961 | 0.540 | −0.89 | −46.04 | 5.50E−02 |
| hsa-let-7f-5p | MIMAT0000067 | 0.543 | −0.88 | −45.66 | 9.44E−02 |
| hsa-miR-548d-5p | MIMAT0004812 | 0.551 | −0.86 | −44.90 | 5.00E−02 |
| hsa-miR-196a-5p | MIMAT0000226 | 0.578 | −0.79 | −42.17 | 3.36E−02 |
| hsa-miR-155-5p | MIMAT0000646 | 0.582 | −0.78 | −41.76 | 9.93E−02 |
| hsa-miR-136-5p | MIMAT0000448 | 0.586 | −0.77 | −41.36 | 3.36E−02 |
| hsa-miR-4421 | MIMAT0018934 | 0.586 | −0.77 | −41.36 | 9.25E−02 |
| hsa-miR-532-3p | MIMAT0004780 | 0.611 | −0.71 | −38.87 | 9.48E−02 |
| hsa-miR-99b-5p | MIMAT0000689 | 0.633 | −0.66 | −36.71 | 8.79E−02 |
| hsa-miR-655-3p | MIMAT0003331 | 0.646 | −0.63 | −35.38 | 8.61E−02 |
| hsa-miR-587 | MIMAT0003253 | 0.660 | −0.6 | −34.02 | 9.10E−02 |
| hsa-miR-452-5p | MIMAT0001635 | 0.669 | −0.58 | −33.10 | 8.73E−02 |
| hsa-miR-548v | MIMAT0015020 | 0.693 | −0.53 | −30.74 | 9.24E−02 |

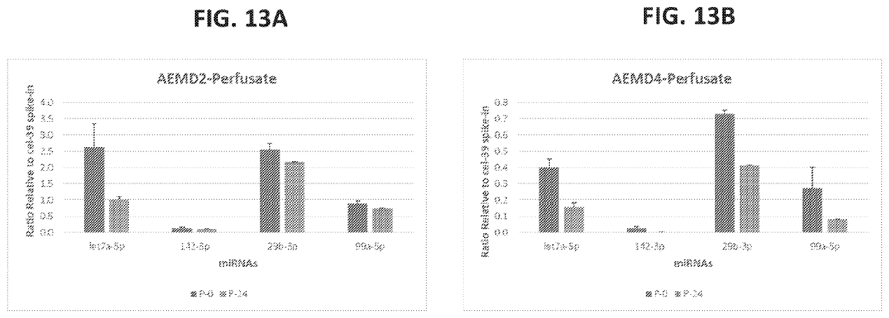

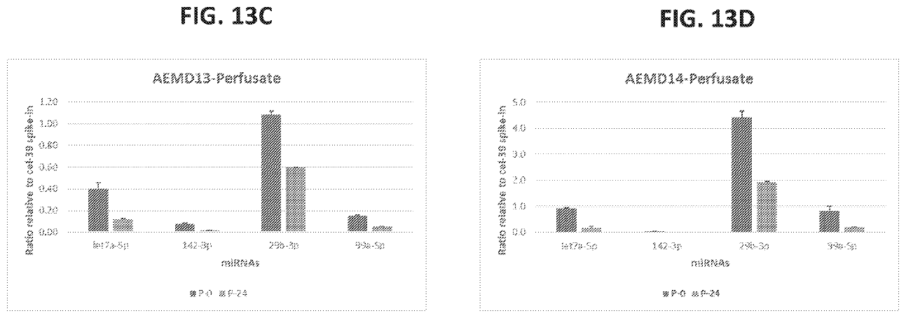

To further confirm the depletion of miRNAs using the devices disclosed herein, a quantitative measurement of 4 miRNAs that were depleted in the kidney perfusates was performed using qPCR. For this method, a cel-miR-39 miRNA marker was spiked into 200 μl of kidney perfusates during total RNA isolation using the Plasma/Serum RNA purification Mini Kit (Norgen Biotek Corp, Thorold, Canada). 2 μl of purified RNA was then reverse transcribed using the Taqman Advanced cDNA Synthesis Kit (Thermofisher) to produce cDNA templates. The miRNA targets of interest were amplified by qPCR on a Quant 3 Studio instrument by mixing one 1 ul of cDNA template in a 20 μl reaction with the Taqman Fast Advanced Master Mix (Thermofisher) and Taqman Advanced miRNA primers. The reaction conditions included an initial denaturation at 95° C. for 2 min, and then 40 cycles of denaturing at 95° C. for 3 s and annealing/extension at 60° C. for 30 s with target amplification data collected on the Quant 3 Studio instrument. The delta C(T) method was used to normalize the relative level of each miRNA target to the level of the cel-miR-39 spike-in used in each sample. Since the quantity of cel-miR-39 miRNA remained constant in each sample, the relative changes in the abundance of other miRNA targets were attributed to a reduction by treatments using the devices as depicted in FIGS. 11-12. As shown in FIG. 13, all 4 miRNAs (hsa-miR-let7a-5p, hsa-miR-142-3p, hsa-miR-29b-3p, and hsa-miR-99a-5p) showed a depletion after treatment. The average depletion observed for each miRNA target evaluated in these treatments was as follows: hsa-let-7a-5p (68±9%), hsa-miR-142-3p (70±37%), hsa-miR-29b-3p (40±18%), and/or hsa-miR-99a-5p (57±29%).

### Examples 7: Removal of Chemokines, Cytokines and Proteins from Kidney Perfusate

This example describes methods of removal of chemokines, cytokines and proteins from a kidney perfusate and can be used in addition to performing examples disclosed herein or individually.

For this example, discarded kidneys were perfused using normothermic ex vivo machine perfusion. Then 250 milliliters of kidney perfusate was put into a circuit (FIG. 11 or FIG. 12) containing a full-scale Hemopurifier®, filled with 40 grams of GNA affinity resin. The perfusate was circulated at 200 milliliters per minute for a total of 24 total volume passes. 500 microliters of pre- and post-treated kidney perfusate were then submitted for analysis. Immune protein levels were quantified using bead panels; Human Cytokine/Chemokine 96-Plex Discovery Assay, Human Complement 13-Plex Discovery Assay, and Human Supplemental Biomarker 10-Plex Discovery Assay Chemokine (Eve Technologies, Calgary, Canada) according to the manufacturer's protocol. The data was loaded into the R statistical environment (v4.3.1). Statistical analysis of experimental data was performed using R programing language (v4.3.2) and the integrated development environment RStudio. R and RStudio were used for data preprocessing, exploratory data analysis, hypothesis testing, and data visualization. A list of chemokines, cytokines and proteins depleted using devices as depicted in FIG. 11 or FIG. 12 are shown in Tables 5-8. Table 5 shows a list of chemokines, cytokines and proteins depleted using a normal flow device as depicted in FIG. 11. Table 6 shows a list of chemokines, cytokines and proteins depleted using a normal flow device as depicted in FIG. 11 with a p<0.1. Table 7 shows a list of chemokines and cytokines depleted using a side port flow device as depicted in FIG. 11. Table 8 shows a list of chemokines, cytokines and proteins depleted using a side port flow device as depicted in FIG. 11 with p<0.1.

TABLE 5
Chemokine and Cytokine Depleted using GNA Resin Normal flow direction.

| | | GNA Resin - Normal Flow | | | |
|---|---|---|---|---|---|
| | | | Log2 | | |
| | | Fold | Fold | Percent | |
| Protein | Gene ID | Change | Change | Change | p-value |
| Complement C2 | C2 | 0.23 | −2.12 | −77% | 1.56E−03 |
| Complement C4b | C4B | 0.17 | −2.59 | −83% | 1.02E−03 |
| Complement C5 | C5 | 0.73 | −0.46 | −27% | 0.24 |
| Mannose Binding Lectin | LMAN1 | 0.46 | −1.13 | −54% | 0.18 |
| Complement C1q | C1QBP | 0.32 | −1.63 | −68% | 1.42E−02 |
| BDNF | BDNF | 0.52 | −0.93 | −48% | 5.69E−03 |
| Cathepsin D | CTSD | 0.25 | −1.99 | −75% | 0.17 |
| NCAM | NCAM | 0.94 | −0.09 | −6% | 0.87 |
| PAI-1 (Total) | SERPINE1 | 0.61 | −0.72 | −39% | 0.50 |
| PDGF-AA | PDGFA | 0.83 | −0.27 | −17% | 0.37 |
| PDGF-AB/BB | PDGFB | 0.55 | −0.85 | −45% | 2.19E−03 |
| RANTES | CCLS | 0.23 | −2.13 | −77% | 2.10E−03 |
| sICAM-1 | ICAM1 | 0.83 | −0.27 | −17% | 0.53 |
| sVCAM-1 | VCAM1 | 0.52 | −0.94 | −48% | 6.55E−02 |
| FGF-2 | FGF2 | 0.55 | −0.87 | −45% | 0.28 |
| Fractalkine | CX3CL1 | 0.13 | −2.99 | −87% | 1.56E−02 |
| GROα | CXCL1 | 0.85 | −0.23 | −15% | 0.81 |
| IFNα2 | IFNA2 | 0.03 | −4.89 | −97% | 5.95E−02 |
| IL-1RA | IL1RN | 0.92 | −0.12 | −8% | 0.81 |
| IL-2 | IL2 | 0.22 | −2.21 | −78% | 0.35 |
| IL-3 | IL3 | 0.07 | −3.87 | −93% | 0.35 |
| IL-4 | IL4 | 0.85 | −0.23 | −15% | 0.67 |
| IL-8 | CXCL8 | 0.97 | −0.05 | −3% | 0.96 |
| IL-9 | IL9 | 0.61 | −0.72 | −39% | 1.94E−03 |
| IL-10 | IL10 | 0.96 | −0.06 | −4% | 0.93 |
| IL-12p40 | IL12B | 0.00 | −10.97 | −100% | 9.52E−02 |
| IL-12p70 | IL12 | 0.75 | −0.41 | −25% | 0.12 |
| IL-13 | IL13 | 0.57 | −0.80 | −43% | 0.38 |
| IL-15 | IL15 | 0.58 | −0.79 | −42% | 6.45E−02 |
| IP-10 | CXCL10 | 0.82 | −0.29 | −18% | 0.80 |
| MCP-1 | CCL2 | 0.71 | −0.49 | −29% | 0.53 |
| MCP-3 | CCL7 | 0.38 | −1.38 | −62% | 0.21 |
| MDC | CCL22 | 0.32 | −1.64 | −68% | 0.16 |
| MIP-1β | CCL4 | 0.00 | −13.79 | −100% | 3.66E−07 |
| PDGF-AA | PDGFA | 0.64 | −0.64 | −36% | 0.42 |
| RANTES | CCL5 | 0.29 | −1.80 | −71% | 3.03E−03 |
| sCD40L | CD40LG | 0.91 | −0.13 | −9% | 0.91 |
| TNFα | TNF | 0.81 | −0.31 | −19% | 0.60 |
| TNFβ | LTA | 0.03 | −5.25 | −97% | 0.14 |
| 6CKine | CCL21 | 0.00 | −16.87 | −100% | 1.26E−08 |
| APRIL | TNFSF13 | 0.01 | −6.27 | −99% | 5.72E−03 |
| BCA-1 | CXCL13 | 0.06 | −4.03 | 94% | 2.82E−02 |
| CCL28 | CCL28 | 0.56 | −0.83 | −44% | 0.45 |
| CTACK | CCL27 | 0.00 | −11.21 | −100% | 2.31E−02 |
| CXCL16 | CXCL16 | 0.96 | −0.07 | −4% | 0.80 |
| ENA-78 | CXCL5 | 0.15 | −2.78 | −85% | 1.17E−07 |
| Eotaxin-2 | CCL24 | 0.75 | −0.42 | −25% | 0.72 |
| Eotaxin-3 | CCL26 | 0.00 | −12.07 | −100% | 8.26E−03 |
| GCP-2 | TUBGCP2 | 0.26 | −1.93 | −74% | 1.37E−02 |
| Granzyme A | GZMA | 0.23 | −2.14 | −77% | 0.16 |
| Granzyme B | GZMB | 0.39 | −1.34 | −61% | 0.33 |
| HMGB1 | HMGB1 | 0.45 | −1.16 | −55% | 0.29 |
| I-TAC | CXCL11 | 0.61 | −0.72 | −39% | 0.32 |
| IFNβ | IFNB1 | 0.00 | −7.68 | −100% | 3.15E−02 |
| IFNω | IFNW1 | 0.04 | −4.76 | −96% | 0.35 |
| IL-11 | IL11 | 0.00 | −7.75 | −100% | 0.15 |
| IL-16 | IL16 | 0.23 | −2.13 | −77% | 9.66E−04 |
| IL-20 | IL20 | 0.17 | −2.58 | −83% | 6.63E−03 |
| IL-24 | IL24 | 0.35 | −1.53 | −65% | 0.23 |
| IL-31 | IL31 | 0.18 | −2.48 | −82% | 1.45E−03 |
| IL-33 | IL33 | 0.18 | −2.51 | −82% | 4.38E−02 |
| IL-34 | IL34 | 0.35 | −1.53 | −65% | 8.07E−03 |
| IL-35 | IL12A | 0.60 | −0.73 | −40% | 0.31 |
| LIF | LIF | 0.52 | −0.95 | −48% | 0.37 |
| MCP-2 | CCL8 | 0.39 | −1.37 | −61% | 8.73E−02 |
| MCP-4 | CCL13 | 0.40 | −1.32 | −60% | 0.11 |
| MIP 1δ | CCL15 | 0.76 | −0.40 | −24% | 0.12 |
| MIP-3α | CCL20 | 0.11 | −3.21 | −89% | 7.97E−02 |
| MIP-3β | CCL19 | 0.03 | −5.18 | −97% | 2.19E−02 |
| MPIF-1 | CCL23 | 0.65 | −0.62 | −35% | 0.24 |
| Perforin | PRF1 | 0.19 | −2.40 | −81% | 5.06E−02 |
| sCD137 | TNFRSF9 | 0.91 | −0.13 | −9% | 0.72 |
| SDF-1 | CXCL12 | 0.13 | −2.91 | −87% | 5.47E−05 |
| sFas | FAS | 0.71 | −0.50 | −29% | 0.35 |
| sFasL | FASLG | 0.96 | −0.06 | −4% | 0.86 |
| TARC | CCL17 | 0.23 | −2.15 | −77% | 7.47E−02 |
| TPO | TPO | 0.07 | −3.90 | −93% | 1.41E−02 |
| TRAIL | TNFSF10 | 0.99 | −0.01 | −1% | 0.99 |

TABLE 6
Chemokine/Cytokines Significantly Depleted
using GNA Resin Normal flow direction.

| | | GNA Resin - Normal Flow | | | |
|---|---|---|---|---|---|
| | | | Log2 | | |
| | | Fold | Fold | Percent | |
| Protein | Gene ID | Change | Change | Change | p-value |
| Complement C2 | C2 | 0.23 | −2.12 | −77% | 1.56E−03 |
| Complement C4b | C4B | 0.17 | −2.59 | −83% | 1.02E−03 |
| Complement C1q | C1QBP | 0.32 | −1.63 | −68% | 1.42E−02 |
| BDNF | BDNF | 0.52 | −0.93 | −48% | 5.69E−03 |
| PDGF-AB/BB | PDGFB | 0.55 | −0.85 | −45% | 2.19E−03 |
| RANTES | CCL5 | 0.23 | −2.13 | −77% | 2.10E−03 |
| sVCAM-1 | VCAM1 | 0.52 | −0.94 | −48% | 6.55E−02 |
| Fractalkine | CX3CL1 | 0.13 | −2.99 | −87% | 1.56E−02 |
| IFNα2 | IFNA2 | 0.03 | −4.89 | −97% | 5.95E−02 |
| IL-9 | IL9 | 0.61 | −0.72 | −39% | 1.94E−03 |
| IL-12p40 | IL12B | 0.00 | −10.97 | −100% | 9.52E−02 |
| IL-15 | IL15 | 0.58 | −0.79 | −42% | 6.45E−02 |
| MIP-1β | CCL4 | 0.00 | −13.79 | −100% | 3.66E−07 |
| RANTES | CCL5 | 0.29 | −1.80 | −71% | 3.03E−03 |
| 6CKine | CCL21 | 0.00 | −16.87 | −100% | 1.26E−08 |
| APRIL | TNFSF13 | 0.01 | −6.27 | −99% | 5.72E−03 |
| BCA-1 | CXCL13 | 0.06 | −4.03 | −94% | 2.82E−02 |
| CTACK | CCL27 | 0.00 | −11.21 | −100% | 2.31E−02 |
| ENA-78 | CXCL5 | 0.15 | −2.78 | −85% | 1.17E−07 |
| Eotaxin-3 | CCL26 | 0.00 | −12.07 | −100% | 8.26E−03 |
| GCP-2 | TUBGCP2 | 0.26 | −1.93 | −74% | 1.37E−02 |
| IFNβ | IFNB1 | 0.00 | −7.68 | −100% | 3.15E−02 |
| IL-16 | IL16 | 0.23 | −2.13 | −77% | 9.66E−04 |
| IL-20 | IL20 | 0.17 | −2.58 | −83% | 6.63E−03 |
| IL-31 | IL31 | 0.18 | −2.48 | −82% | 1.45E−03 |
| IL-33 | IL33 | 0.18 | −2.51 | −82% | 4.38E−02 |
| IL-34 | IL34 | 0.35 | −1.53 | −65% | 8.07E−03 |
| MCP-2 | CCL8 | 0.39 | −1.37 | −61% | 8.73E−02 |
| MIP-3α | CCL20 | 0.11 | −3.21 | −89% | 7.97E−02 |
| MIP-3β | CCL19 | 0.03 | −5.18 | −97% | 2.19E−02 |
| Perforin | PRF1 | 0.19 | −2.40 | −81% | 5.06E−02 |
| SDF-1 | CXCL12 | 0.13 | −2.91 | −87% | 5.47E−05 |
| TARC | CCL17 | 0.23 | −2.15 | −77% | 7.47E−02 |
| TPO | TPO | 0.07 | −3.90 | −93% | 1.41E−02 |

TABLE 7
Chemokine/Cytokines Depleted using GNA Resin Side Port Flow Direction

| | | GNA Resin - Side Port Flow | | | |
|---|---|---|---|---|---|
| | | | Log2 | | |
| | | Fold | Fold | Percent | |
| Protein | Gene ID | Change | Change | Change | p-value |
| Complement C2 | C2 | 0.41 | −1.29 | −59% | 4.26E−02 |
| Complement C4b | C4B | 0.11 | −3.18 | −89% | 2.68E−06 |
| Complement C5 | C5 | 0.70 | −0.51 | −30% | 0.23 |
| Mannose Binding Lectin | LMAN1 | 0.43 | −1.22 | −57% | 0.16 |
| Complement C1q | C1QBP | 0.20 | −2.30 | −80% | 8.95E−04 |
| Complement C4 | C4A | 0.93 | −0.10 | −7% | 0.49 |
| BDNF | BDNF | 0.47 | −1.10 | −53% | 4.50E−03 |
| Cathepsin D | CTSD | 0.26 | −1.92 | −74% | 0.18 |
| Myeloperoxidase (MPO) | MPO | 0.97 | −0.05 | −3% | 0.94 |
| NCAM | NCAM | 0.99 | −0.01 | −1% | 0.98 |
| PAI-1 (Total) | SERPINE1 | 0.61 | −0.72 | −39% | 0.49 |
| PDGF-AA | PDGFA | 0.76 | −0.39 | −24% | 0.19 |
| PDGF-AB/BB | PDGFB | 0.44 | −1.20 | −56% | 2.866−04 |
| RANTES | CCL5 | 0.27 | −1.90 | −73% | 2.84E−03 |
| sICAM-1 | ICAM1 | 0.84 | −0.26 | −16% | 0.56 |
| sVCAM-1 | VCAM1 | 0.53 | −0.93 | −47% | 5.42E−02 |
| Eotaxin | CCL11 | 0.93 | −0.11 | −7% | 0.59 |
| FGF-2 | FGF2 | 0.49 | −1.02 | −51% | 0.21 |
| Fractalkine | CX3CL1 | 0.13 | −2.89 | −87% | 1.47E−02 |
| GROα | CXCL1 | 0.78 | −0.35 | −22% | 0.71 |
| IFNα2 | IFNA2 | 0.00 | −9.08 | −100% | 5.33E−02 |
| IFNγ | IFNG | 0.06 | −3.97 | −94% | 0.17 |
| IL-1α | IL1A | 0.82 | −0.29 | −18% | 0.59 |
| IL-1RA | IL1RN | 0.75 | −0.41 | −25% | 0.36 |
| IL-2 | IL2 | 0.22 | −2.21 | −78% | 0.35 |
| IL-3 | IL3 | 0.07 | −3.87 | −93% | 0.35 |
| IL-4 | IL4 | 0.71 | −0.48 | −29% | 0.43 |
| IL-6 | IL6 | 0.99 | −0.01 | −1% | 0.99 |
| IL-8 | CXCL8 | 0.94 | −0.09 | −6% | 0.93 |
| IL-9 | 119 | 0.53 | −0.92 | −47% | 1.80E−02 |
| IL-10 | IL10 | 0.98 | −0.03 | −2% | 0.96 |
| IL-12p40 | IL12B | 0.95 | −0.07 | −5% | 0.96 |
| IL-13 | IL13 | 0.36 | −1.46 | −64% | 0.21 |
| IL-15 | IL15 | 0.62 | −0.69 | −38% | 0.14 |
| IP-10 | CXCL10 | 0.64 | −0.64 | −36% | 0.56 |
| MCP-1 | CCL2 | 0.68 | −0.56 | −32% | 0.46 |
| MCP-3 | CCL7 | 0.00 | −11.01 | −100% | 1.27E−03 |
| MDC | CCL22 | 0.27 | −1.88 | −73% | 0.12 |
| MIP-1β | CCL4 | 0.00 | −13.79 | −100% | 3.66E−07 |
| PDGF-AA | PDGFA | 0.54 | −0.89 | −46% | 0.30 |
| RANTES | CCL5 | 0.30 | −1.76 | −70% | 2.91E−03 |
| sCD40L | CD40LG | 0.98 | −0.04 | −2% | 0.98 |
| TNFα | TNF | 0.78 | −0.36 | −22% | 0.53 |
| TNFβ | LTA | 0.03 | −5.25 | −97% | 0.14 |
| 6CKine | CCL21 | 0.11 | −3.18 | −89% | 2.35E−03 |
| APRIL | TNFSF13 | 0.01 | −6.56 | −99% | 5.65E−03 |
| BAFF | TNFSF13B | 1.00 | −0.01 | 0% | 0.99 |
| BCA-1 | CXCL13 | 0.07 | −3.84 | −93% | 2.92E−02 |
| CCL28 | CCL28 | 0.19 | −2.39 | −81% | 6.96E−02 |
| CTACK | CCL27 | 0.00 | −11.21 | −100% | 2.31E−02 |
| ENA-78 | CXCL5 | 0.16 | −2.66 | −84% | 5.17E−07 |
| Eotaxin-2 | CCL24 | 0.70 | −0.51 | −30% | 0.68 |
| Eotaxin-3 | CCL26 | 0.00 | −12.07 | −100% | 8.26E−03 |
| GCP-2 | TUBGCP2 | 0.25 | −2.00 | −75% | 1.09E−02 |
| Granzyme A | GZMA | 0.26 | −1.96 | −74% | 0.18 |
| Granzyme B | GZMB | 0.36 | −1.47 | −64% | 0.30 |
| HMGB1 | HMGB1 | 0.31 | −1.69 | −69% | 0.15 |
| I-309 | CCL1 | 0.60 | −0.74 | −40% | 0.16 |
| I-TAC | CXCL11 | 0.40 | −1.33 | −60% | 0.11 |
| IFNβ | IFNB1 | 0.00 | −7.68 | −100% | 3.15E−02 |
| IFNω | IFNW1 | 0.04 | −4.76 | −96% | 0.35 |
| IL-11 | IL11 | 0.14 | −2.87 | −86% | 0.21 |
| IL-16 | IL16 | 0.30 | −1.75 | −70% | 1.86E−03 |
| IL-20 | IL20 | 0.22 | −2.18 | −78% | 9.46E−03 |
| IL-24 | IL24 | 0.20 | −2.30 | −80% | 8.71E−02 |
| IL-31 | IL31 | 0.08 | −3.69 | −92% | 6.64E−04 |
| IL-33 | IL33 | 0.19 | −2.39 | −81% | 4.71E−02 |
| IL-34 | IL34 | 0.39 | −1.34 | −61% | 9.45E−02 |
| IL-35 | IL12A | 0.56 | −0.84 | −44% | 0.27 |
| LIF | LIF | 0.45 | −1.14 | −55% | 0.31 |
| MCP-2 | CCL8 | 0.37 | −1.43 | −63% | 5.95E−02 |
| MCP-4 | CCL13 | 0.35 | −1.52 | −65% | 6.76E−02 |
| MIP 1δ | CCL15 | 0.74 | −0.43 | −26% | 0.10 |
| MIP-3α | CCL20 | 0.09 | −3.47 | −91% | 7.37E−02 |
| MIP-3β | CCL19 | 0.02 | −5.57 | −98% | 2.13E−02 |
| MPIF-1 | CCL23 | 0.66 | −0.59 | −34% | 0.27 |
| Perforin | PRF1 | 0.17 | −2.54 | −83% | 4.57E−02 |
| sCD137 | TNFRSF9 | 0.97 | −0.05 | −3% | 0.90 |
| SDF-1 | CXCL12 | 0.14 | −2.79 | −86% | 9.57E−05 |
| sFas | FAS | 0.68 | −0.55 | −32% | 0.27 |
| sFasL | FASLG | 0.92 | −0.11 | −8% | 0.76 |
| TARC | CCL17 | 0.20 | −2.34 | −80% | 6.46E−02 |
| TPO | TPO | 0.11 | −3.14 | −89% | 1.76E−02 |
| TRAIL | TNFSF10 | 0.95 | −0.08 | −5% | 0.90 |

TABLE 8
Chemokine/Cytokines Significantly Depleted
using GNA Resin Side Port Flow Direction.

| | | GNA Resin - Side Port Flow | | | |
|---|---|---|---|---|---|
| | | | Log2 | | |
| | | Fold | Fold | Percent | |
| Protein | Gene ID | Change | Change | Change | p-value |
| Complement C2 | C2 | 0.41 | −1.29 | −59% | 4.26E−02 |
| Complement C4b | C4B | 0.11 | −3.18 | −89% | 2.68E−06 |
| Complement C1q | C1QBP | 0.20 | −2.30 | −80% | 8.95E−04 |
| BDNF | BDNF | 0.47 | −1.10 | −53% | 4.50E−03 |
| PDGF-AB/BB | PDGFB | 0.44 | −1.20 | −56% | 2.86E−04 |
| RANTES | CCL5 | 0.27 | −1.90 | −73% | 2.84E−03 |
| sVCAM-1 | VCAM1 | 0.53 | −0.93 | −47% | 5.42E−02 |
| Fractalkine | CX3CL1 | 0.13 | −2.89 | −87% | 1.47E−02 |
| IFNα2 | IFNA2 | 0.00 | −9.08 | −100% | 5.33E−02 |
| IL-9 | IL9 | 0.53 | −0.92 | −47% | 1.80E−02 |
| MCP-3 | CCL7 | 0.00 | −11.01 | −100% | 1.27E−03 |
| MIP-1β | CCL4 | 0.00 | −13.79 | −100% | 3.66E−07 |
| RANTES | CCL5 | 0.30 | −1.76 | −70% | 2.91E−03 |
| 6CKine | CCL21 | 0.11 | −3.18 | −89% | 2.35E−03 |
| APRIL | TNFSF13 | 0.01 | −6.56 | −99% | 5.65E−03 |
| BCA-1 | CXCL13 | 0.07 | −3.84 | −93% | 2.92E−02 |
| CCL28 | CCL28 | 0.19 | −2.39 | −81% | 6.96E−02 |
| CTACK | CCL27 | 0.00 | −11.21 | −100% | 2.31E−02 |
| ENA-78 | CXCL5 | 0.16 | −2.66 | −84% | 5.17E−07 |
| Eotaxin-3 | CCL26 | 0.00 | −12.07 | −100% | 8.26E−03 |
| GCP-2 | TUBGCP2 | 0.25 | −2.00 | −75% | 1.09E−02 |
| IFNβ | IFNB1 | 0.00 | −7.68 | −100% | 3.15E−02 |
| IL-16 | IL16 | 0.30 | −1.75 | −70% | 1.86E−03 |
| IL-20 | IL20 | 0.22 | −2.18 | −78% | 9.46E−03 |
| IL-24 | IL24 | 0.20 | −2.30 | −80% | 8.71E−02 |
| IL-31 | IL31 | 0.08 | −3.69 | −92% | 6.64E−04 |
| IL-33 | IL33 | 0.19 | −2.39 | −81% | 4.71E−02 |
| IL-34 | IL34 | 0.39 | −1.34 | −61% | 9.45E−02 |
| MCP-2 | CCL8 | 0.37 | −1.43 | −63% | 5.95E−02 |
| MCP-4 | CCL13 | 0.35 | −1.52 | −65% | 6.76E−02 |
| MIP-3α | CCL20 | 0.09 | −3.47 | −91% | 7.37E−02 |
| MIP-3β | CCL19 | 0.02 | −5.57 | −98% | 2.13E−02 |
| Perforin | PRF1 | 0.17 | −2.54 | −83% | 4.57E−02 |
| SDF-1 | CXCL12 | 0.14 | −2.79 | −86% | 9.57E−05 |
| TARC | CCL17 | 0.20 | −2.34 | −80% | 6.46E−02 |
| TPO | TPO | 0.11 | −3.14 | −89% | 1.76E−02 |

### Examples 8: Analysis of DNA from Kidney Perfusate after Treatment with Exemplary Devices Disclosed Herein

This example describes methods for analysis of DNA from a kidney perfusate and can be used in addition to performing examples disclosed herein or individually.

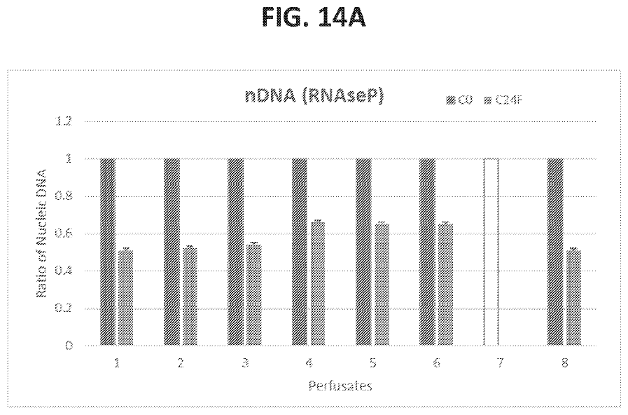

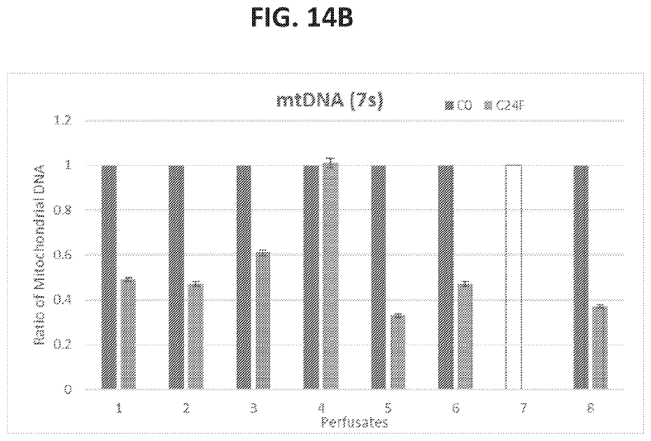

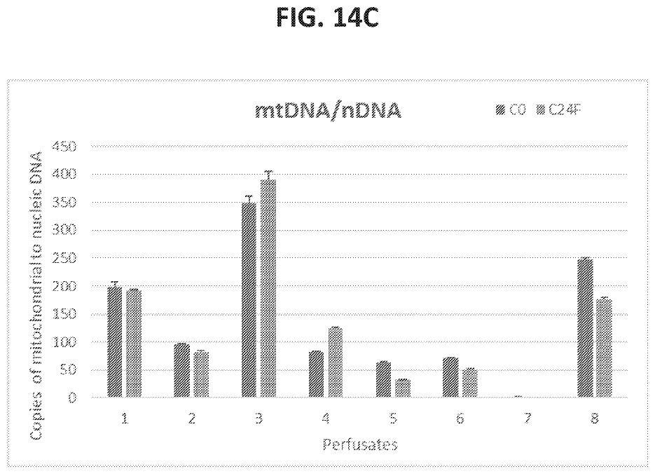

To analyze the DNA content of the kidney perfusates after treatment with the exemplary devices, quantitative measurement of nucleic DNA marker (RNAseP) and mitochondrial DNA marker (7 s) in the kidney perfusates was performed using qPCR. For this method, DNA was purified from the control and kidney perfusate solutions passed for 24 times through the device as depicted in FIG. 10 using the QIAamp DNA Blood mini kit (Qiagen) according to the instructions provided by the manufacturer. In brief, 200 ul of kidney perfusate solution or control was lysed, heated at 56° C. for 10 min, loaded onto a spin column, washed, and then and eluted into 50 ul of the kit's AE elution buffer. DNA purity and concentrations were assessed using the Nanodrop A260. The DNA isolated from the control and kidney perfusate solutions was analyzed using the Quant Studio 3 instrument to evaluate differences in the levels of several markers of interest between perfusates and after HP treatments. A multiplex assay was used to simultaneously amplify both nucleic and mitochondrial DNA in each sample with the Taqman Fast Advanced Master Mix (Thermofisher). Nucleic DNA was amplified using the RNAseP Taqman primers (Thermofisher) while mitochondrial DNA was amplified with the Taqman human 7 s primers (Thermofisher). qPCR was conducted in a 20 μl total volume containing 2 ul of the purified perfusate DNA as a template and the following conditions: initial denaturation at 95° C. for 2 min, and then 40 cycles of denaturing at 95° C. for 3 s and annealing/extension at 60° C. for 30 s. The delta C(T) method was used to quantify relative changes in the content of nucleic and mitochondrial targets in different perfusate samples, as well as the abundance of mitochondrial/nucleic DNA within each perfusate. As shown in FIGS. 14A and 14B, both the nuclear and mitochondrial DNA showed a reduction after treatments with a column packed with a lectin affinity resin (e.g., GNA) followed by filtration through a 0.2 μm membrane. The ratio of mitochondrial DNA (mtDNA)/nuclear DNA (nDNA) is shown in FIG. 14C.

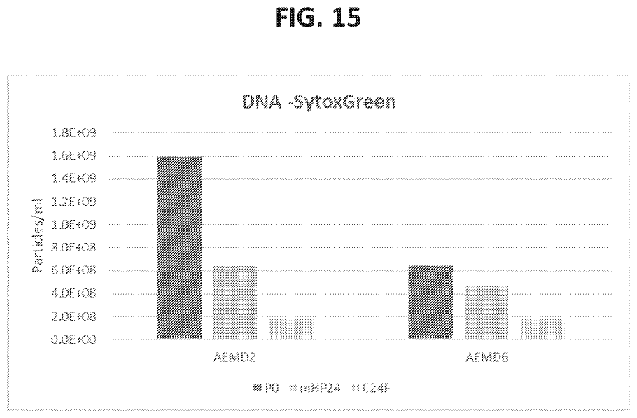

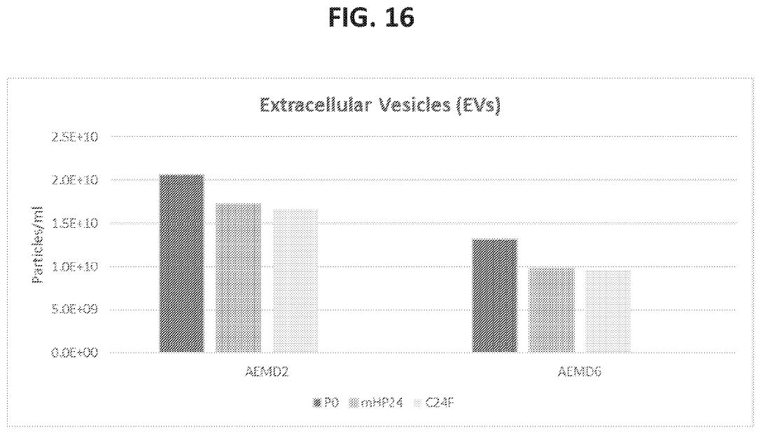

The DNA content of the kidney perfusates after treatment with the exemplary devices was further quantified using SyTox Green DNA staining. A quantitation of extracellular vesicles in the perfusate treated with devices as depicted in FIGS. 8-10 and control was also performed using MemGlow staining of the vesicles. Sytox green staining was always performed in conjunction with EV staining for simultaneous tracking of both particles. Co-staining for concentration and size quantification of EV associated DNA particles was performed by first mixing samples directly with 2 uM MemGlow 640 nm at a 1:1 ratio for quantification of EV particles. 2 uL of MemGlow stained sample were diluted into 98 uL of RO water and stained with Invitrogen™ SYTOX™ Green Ready Flow™ Reagent (REF: R37168), a cell-impermeant nucleic acid dye. The nucleic dye itself was diluted 10-fold, used to stain at a ratio of 1:1 with the diluted MemGlow stained sample and incubated at room temperature for 15 minutes prior to analysis on the NanoFCM instrument. As shown in FIG. 15, treatment of the kidney perfusate using devices as depicted in FIGS. 8-10 removed a majority of DNA particles from the perfusate. In addition, as shown in FIG. 16, a reduction in the number of extracellular vesicles was also observed after treatment of the perfusates using devices as depicted in FIGS. 8-10.

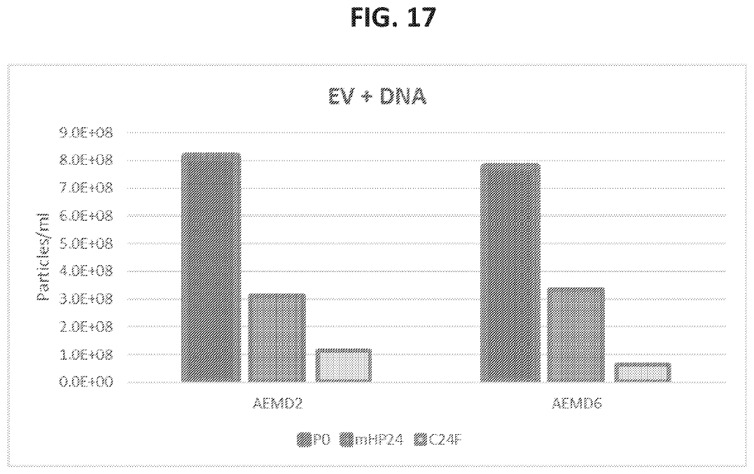

To demonstrate if the DNA particles that were depleted in the kidney perfusates after treatment with the exemplary devices as disclosed herein, Sytox green staining was performed in conjunction with EV staining for simultaneous tracking of both particles using the method as described above. As shown in FIG. 17, it was observed that approximately half of the DNA particles in the kidney perfusates are associated with EVs and treatments with devices as depicted in FIGS. 8-10 reduced 90% of these particles.

### Example 9: Removal of miRNAs in an Organ, Cell or a Tissue Donor Prior to Removal of the Organ, Cell or a Tissue

This example pertains to methods of removal of miRNAs from an organ, cell or a tissue donor prior to removal of the organ, a population of cells or a tissue from the donor. This example can be used in addition to performing examples disclosed herein or individually. Procedures for organ, cell or tissue donors prior to treatment with a lectin affinity hemofiltration device, as described herein: Samples will be collected for pre-treatment assessment of total exosomes, miRNAs or fragments thereof. The results of the pre-treatment can be used for later comparison of total exosomes, miRNAs or fragments thereof present in the patient's circulatory system prior to treatment and in the post-treatment sample. Also, a pre-treatment blood and/or plasma sample may be used for detection of cytokines/inflammatory and other blood biomarkers of organ damage. In preparation for therapy, a hemodialysis catheter will be placed into the donor.

### Example 10: Removal of miRNAs in an Organ, Cell or a Tissue Donor Prior in a Perfused Organ

This example describes methods of removal of miRNAs or fragments thereof from a perfused organ and can be used in addition to performing examples disclosed herein or individually.

Procedures for a perfused organ prior to treatment with a lectin affinity hemofiltration device as described herein. The explanted donor organ will be connected directly to the Hemopurifier® filter or a packed column containing GNA affinity resin. Typically, a pump will supply perfusion fluid via the artery supplying the organ. The perfusion fluid will then exit the organ via the vein and then enter the Hemopurifier® or a column packed with a lectin affinity resin (e.g., GNA) for removal of total exosomes, miRNAs or fragments thereof before being returned to the artery supplying the organ. Perfusion buffer will be collected for pre-treatment assessment of total exosomes, miRNAs or fragments thereof. The results of the pre-treatment can be used for later comparison of total exosomes, miRNAs or fragments thereof present in the patient's circulatory system prior to treatment and in the post-treatment sample. Also, a pre-treatment perfusion buffer sample may be used for detection of cytokines/inflammatory and other blood biomarkers of organ damage.

### Example 11: Removal of miRNAs in an Organ, Cell or a Tissue Donor Prior in a Transplant Recipient

This example describes methods of removal of miRNAs or fragments thereof in a transplant recipient. This example can be used in addition to performing examples disclosed herein or individually.

Procedures for transplant recipient prior to treatment with a lectin affinity hemofiltration device as described herein: Samples will be collected for pre-treatment assessment of total exosomes, miRNAs or fragments thereof. The results of the pre-treatment can be used for later comparison of total exosomes, miRNAs or fragments thereof present in the patient's circulatory system prior to treatment and in the post-treatment sample. Also, a pre-treatment blood and/or plasma sample may be used for detection of cytokines/inflammatory and other blood biomarkers of organ damage. In preparation for treatment, a hemodialysis catheter will be placed into the recipient.

### Example 12: Molecular Profiling of Extracellular Vesicles, e.g., Exosomes or Ectosomes in an Organ, Cell or a Tissue Donor Prior to Removal of the Organ, Cell or a Tissue

This example pertains to molecular profiling of extracellular vesicles (e.g., exosomes or ectosomes) derived from a subject, wherein the subject is an organ, an organ perfusate, cell or a tissue donor prior to removal of the organ. Briefly, 1 ml of plasma will be collected from the subjects. Extracellular vesicles (e.g., exosomes or ectosomes) will be isolated using size or density isolation, including but not limited to, the IZON qEV original columns. Extracellular vesicles (e.g., exosomes or ectosomes) may be used for additional profiling analyses including protein concentration, nanoparticle counts, tetraspanin profiling, glycan surface profiling, RNA sequencing, mass spectrometry and/or GNA binding affinity. The molecular profiling of extracellular vesicles (e.g., exosomes or ectosomes) isolated from organ, cell or tissue donors may be compared to molecular profiling of extracellular vesicles (e.g., exosomes or ectosomes) from healthy subjects (e.g., recipients without transplant rejection) and/or known diseased subjects (e.g., recipients with transplant rejection).

For the lectin immobilization, three spots per lectin are printed using a Nanoplotter 2.1 piezoelectric printer (GeSIM, Germany) at 14° C. and 45% humidity. For the microarray hybridization microarray slides are submerged in blocking buffer (50 mM ethanolamine, mM sodium borate pH 8.5) for 1 h followed by 3 washes with PBST (PBS with 0.005% Tween) and a final wash with PBS. The microarray slides are then fitted to a 24-well frame (ArrayIt, Sunnyvale, CA). For single color experiments, 1.5 μg (by protein concentration) of labeled sample in a final volume of 100 μl in PBST is added to each subarray. For dual color experiments, 1.5 μg of Cy3-labeled extracellular vesicles, e.g., exosomes are incubated with equal amounts of either Cy5-labeled H9 reference or Cy5-labeled parent membrane in 100 μl total volume PBST. The order of the samples is randomized on a total of two slides for all dual color assays. Labeled glycoprotein standards are hybridized on all slides as controls and no statistical difference in lectin activity will be observed between slides. Samples are hybridized to the lectin microarrays for 2 h at room temperature with gentle rocking. The individual subarrays are then washed with PBST for 5-5 min with a final 10 min wash in PBS. Slides are scanned and analyzed using a GenePix 4300B fluorescent slide scanner (Molecular Devices, Sunnyvale, CA) with GenePix Pro 7 software or scanned and analyzed using a different fluorescent slide scanner.

For nanoparticle counts, samples will be diluted in PBS to a concentration of approximately 10⁸-10⁹ particles/ml and measured using the Nanosight LM10 equipment. At least three 30 second capture videos of different segments of the homogenous sample will be evaluated with the NTA 3.3 software in order to particle quantification and sizing measurements.

For tetraspanin profiling of the samples, they will be incubated on the Exoview tetraspanin microarray chips, stained with fluorescently labelled tetraspanin probes, and then imaged and analyzed using the Exoview R100 imaging platform and software.

### Example 13: Molecular Profiling of Extracellular Vesicles (e.g., Exosomes or Ectosomes) in a Perfused Organ

This example pertains to molecular profiling of extracellular vesicles (e.g., exosomes or ectosomes) derived from a perfused organ. Briefly, the perfusion fluid that exits the organ via the vein into the Hemopurifier® and the perfusion fluid that gets returned to the organ from the Hemopurifier® will be collected. Extracellular vesicles (e.g., exosomes or ectosomes) may be isolated using size or density isolation, including but not limited to, the IZON qEV original columns. Extracellular vesicles (e.g., exosomes or ectosomes) will be used for additional profiling analyses including protein concentration, nanoparticle counts, tetraspanin profiling, glycan surface profiling, RNA sequencing, mass spectrometry and/or GNA binding affinity. The molecular profiling of extracellular vesicles (e.g., exosomes or ectosomes) isolated from the perfused organ prior to Hemopurifier® treatment may be compared to molecular profiling of extracellular vesicles (e.g., exosomes or ectosomes) from perfused organs after Hemopurifier® treatment.

For the lectin immobilization, three spots per lectin are printed using a Nanoplotter 2.1 piezoelectric printer (GeSIM, Germany) at 14° C. and 45% humidity. For the microarray hybridization microarray slides are submerged in blocking buffer (50 mM ethanolamine, mM sodium borate pH 8.5) for 1 h followed by 3 washes with PBST (PBS with 0.005% Tween) and a final wash with PBS. The microarray slides are then fitted to a 24-well frame (ArrayIt, Sunnyvale, CA). For single color experiments, 1.5 μg (by protein concentration) of labeled sample in a final volume of 100 μl in PBST is added to each subarray. For dual color experiments, 1.5 μg of Cy3-labeled extracellular vesicles (e.g., exosomes) are incubated with equal amounts of either Cy5-labeled H9 reference or Cy5-labeled parent membrane in 100 μl total volume PBST. The order of the samples is randomized on a total of two slides for all dual color assays. Labeled glycoprotein standards are hybridized on all slides as controls and no statistical difference in lectin activity will be observed between slides. Samples are hybridized to the lectin microarrays for 2 h at room temperature with gentle rocking. The individual subarrays are then washed with PBST for 5×5 min with a final 10 min wash in PBS. Slides are scanned and analyzed using a GenePix 4300B fluorescent slide scanner (Molecular Devices, Sunnyvale, CA) with GenePix Pro 7 software or scanned and analyzed using a different fluorescent slide scanner.

For nanoparticle counts, samples will be diluted in PBS to a concentration of approximately 10⁸-10⁹ particles/ml and measured using the Nanosight LM10 equipment. At least three 30 second capture videos of different segments of the homogenous sample may be evaluated with the NTA 3.3 software in order to particle quantification and sizing measurements.

For tetraspanin profiling of the samples, they may be incubated on the Exoview tetraspanin microarray chips, stained with fluorescently labelled tetraspanin probes, and then imaged and analyzed using the Exoview R100 imaging platform and software.

### Example 14: Molecular Profiling of Extracellular Vesicles, e.g., Exosomes or Ectosomes in a Transplant Recipient

This example pertains to molecular profiling of extracellular vesicles (e.g., exosomes or ectosomes) derived from a subject, wherein the subject is an organ, cell or a tissue transplant recipient with or without transplant rejection. Briefly, 1 ml of plasma will be collected from the subjects. Extracellular vesicles (e.g., exosomes or ectosomes) will be isolated using size or density isolation, including but not limited to, the IZON qEV original columns. Extracellular vesicles (e.g., exosomes or ectosomes) will be used for additional profiling analyses including protein concentration, nanoparticle counts, tetraspanin profiling, glycan surface profiling, RNA sequencing, mass spectrometry and/or GNA binding affinity. The molecular profiling of extracellular vesicles (e.g., exosomes or ectosomes) isolated from organ, cell or tissue transplant recipients with transplant rejection will be compared to molecular profiling of extracellular vesicles (e.g., exosomes or ectosomes) isolated from organ, cell or tissue transplant recipients without transplant rejection.

For the lectin immobilization, three spots per lectin are printed using a Nanoplotter 2.1 piezoelectric printer (GeSIM, Germany) at 14° C. and 45% humidity. For the microarray hybridization microarray slides are submerged in blocking buffer (50 mM ethanolamine, mM sodium borate pH 8.5) for 1 h followed by 3 washes with PBST (PBS with 0.005% Tween) and a final wash with PBS. The microarray slides are then fitted to a 24-well frame (ArrayIt, Sunnyvale, CA). For single color experiments, 1.5 μg (by protein concentration) of labeled sample in a final volume of 100 μl in PBST is added to each subarray. For dual color experiments, 1.5 μg of Cy3-labeled extracellular vesicles (e.g., exosomes or ectosomes) are incubated with equal amounts of either Cy5-labeled H9 reference or Cy5-labeled parent membrane in 100 μl total volume PBST. The order of the samples is randomized on a total of two slides for all dual color assays. Labeled glycoprotein standards are hybridized on all slides as controls and no statistical difference in lectin activity will be observed between slides. Samples are hybridized to the lectin microarrays for 2 h at room temperature with gentle rocking. The individual subarrays are then washed with PBST for 5×5 min with a final 10 min wash in PBS. Slides are scanned and analyzed using a GenePix 4300B fluorescent slide scanner (Molecular Devices, Sunnyvale, CA) with GenePix Pro 7 software or scanned and analyzed using a different fluorescent slide scanner.

For nanoparticle counts, samples will be diluted in PBS to a concentration of approximately 10⁸-10⁹ particles/ml and measured using the Nanosight LM10 equipment. At least three 30 second capture videos of different segments of the homogenous sample will be evaluated with the NTA 3.3 software in order to particle quantification and sizing measurements.

For tetraspanin profiling of the samples, they will be incubated on the Exoview tetraspanin microarray chips, stained with fluorescently labelled tetraspanin probes, and then imaged and analyzed using the Exoview R100 imaging platform and software.

In at least some of the previously described embodiments, one or more elements used in an embodiment can interchangeably be used in another embodiment unless such a replacement is not technically feasible. It will be appreciated by those skilled in the art that various other omissions, additions, and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those of skill in the art. The various aspects and embodiments disclosed herein are for purposes of illustration and are not intended to be limiting, with the true scope and spirit being indicated by the following claims.

1.-87. (canceled)

88. A method for removing miRNAs from a sample, the method comprising:
contacting a sample comprising miR-2110, miR-320b, miR-130b-3p, and/or miR-484 with a support, which comprises Galanthus nivalis lectin (GNA); and
detecting miR-2110, miR-320b, miR-130b-3p, and/or miR-484 in the sample prior to contacting the sample with the support or after contacting the sample with the support or both.

89. The method of claim 88, wherein the sample is selected from peripheral blood, serum, plasma, organ perfusion fluid or organ perfusate, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, umbilical cord blood, ascites fluid, cell culture, such as CAR T cell culture, stem cell culture, or pancreatic islet culture.

90. The method of claim 89, wherein the sample is peripheral blood or plasma.

91. The method of claim 88, wherein the support is provided in an extracorporeal device.

92. The method of claim 91, wherein after contacting the sample with the support in the extracorporeal device, the sample has a reduced amount of miR-2110, miR-320b, miR-130b-3p, and/or miR-484 as compared to the amount of miR-2110, miR-320b, miR-130b-3p, and/or miR-484 present in the sample prior to contact with the support and the sample having the reduced amount of miR-2110, miR-320b, miR-130b-3p, and/or miR-484 after contact with the support in the extracorporeal device is reintroduced into a subject.

93. The method of claim 88, wherein the support comprises Sepharose, agarose, latex, glass, thermoplastic polymers, polystyrene, polyethylene beads, polyvinyl, diatomaceous earth, nitrocellulose or silicon.

94. The method of claim 88, wherein the GNA is immobilized or adsorbed onto the support.

95. The method of claim 88, wherein the support comprises diatomaceous earth.

96. A method for removing miRNAs from a subject, the method comprising:
a) introducing a sample comprising miR-2110, miR-320b, miR-130b-3p, and/or miR-484 from a subject into an extracorporeal device, comprising a support, which comprises a lectin;
b) contacting the sample comprising miR-2110, miR-320b, miR-130b-3p, and/or miR-484 with the support in the extracorporeal device;
c) reintroducing the sample obtained after b) into said subject, wherein the sample obtained after b) has a reduced amount of miR-2110, miR-320b, miR-130b-3p, and/or miR-484 as compared to the sample of said subject prior to b); and
d) detecting miR-2110, miR-320b, miR-130b-3p, and/or miR-484 in a sample from the subject prior to (a) or after (b) or both.

97. The method of claim 96, wherein the sample is peripheral blood or plasma.

98. The method of claim 96, wherein the lectin is Galanthus nivalis lectin (GNA).

99. The method of claim 96, wherein the extracorporeal device comprises a hollow fiber cartridge comprising the lectin and wherein the sample flows through hollow fibers of the hollow fiber cartridge.

100. The method of claim 99, wherein the hollow fibers of the hollow fiber cartridge comprise a pore size that excludes cellular components of the sample from contacting the lectin.

101. The method of claim 100, wherein the pore size is 200 nm or about 200 nm.

102. The method of claim 96, wherein the method further comprises repeating steps a), b), and c).

103. The method of claim 102, wherein steps a), b), and c) are repeated every day for 1, 2, 3, 4, 5, 6, 7, 8, 9, 10, 11, 12, 13, 14, or 15 days.

104. An extracorporeal device comprising a hollow fiber cartridge comprising Galanthus nivalis agglutinin (GNA) lectin, wherein blood or plasma can flow through hollow fibers of the hollow fiber cartridge, wherein the hollow fibers of the hollow fiber cartridge comprise a pore size that excludes cellular components of the blood or plasma from contacting the lectin, such as a pore size of 20-500 nm or about 20-500 nm or 200 nm or about 200 nm, wherein the lectin is immobilized on to a solid support, and the hollow fiber cartridge comprises the lectin immobilized on the solid support, wherein the solid support preferably comprises diatomaceous earth, further comprising miR-2110, miR-320b, miR-130b-3p, and/or miR-484.
